(19) United States
(12) Patent Application Publication
(10) Pub. No.: US 2026/0202398 A1
(43) Pub. Date: Jul. 16, 2026

(54) POLYPEPTIDE-FUNCTIONALIZING REAGENTS AND METHODS OF USE

(71) Applicant: Quantum-Si Incorporated, Branford, CT (US)

(72) Inventors: Mathivanan Chinnaraj, San Diego, CA (US); John Silvio Vieceli, Carlsbad, CA (US)

(73) Assignee: Quantum-Si Incorporated, Branford, CT (US)

(21) Appl. No.: 19/359,478

(22) Filed: Oct. 15, 2025

Related U.S. Application Data
(60) Provisional application No. 63/707,715, filed on Oct. 15, 2024.

(51) Int. Cl. G01N 33/533; C07K 7/06; C07K 19/00

(52) U.S. Cl. CPC G01N 33/533; C07K 7/06; C07K 19/00; C07K 2319/21; C07K 2319/50; C07K 2319/60

(57) ABSTRACT

Provided herein are compositions and methods for functionalizing a polypeptide for single-molecule analysis. In some aspects, the disclosure provides compositions and methods for immobilizing a peptide barcode to a surface. In some aspects, the disclosure provides compositions and methods for luminescently labeling a protein of interest.

### CROSS REFERENCE TO RELATED APPLICATIONS

This application claims priority under 35 U.S.C. § 119(e) to U.S. Provisional Application No. 63/707,715, filed Oct. 15, 2024, which is hereby incorporated by reference in its entirety.

### REFERENCE TO AN ELECTRONIC SEQUENCE LISTING

The contents of the electronic sequence listing (R070870181US01-SEQ-KVC.xml; Size: 62,343 bytes; and Date of Creation: Oct. 15, 2025) are herein incorporated by reference in its entirety.

### BACKGROUND

Measurements of the proteome provide deep and valuable insight into biological processes. However, the complex nature of the proteome and the chemical properties of proteins present fundamental challenges to achieving sensitivity, throughput, cost, and adoption on par with DNA sequencing technologies. Methods to directly sequence single protein molecules offer the maximum possible detection sensitivity, with the potential to enable single-cell inputs, digital quantification based on read counts, detection of posttranslational modifications (PTMs) and low-abundance or aberrant proteoforms, and cost and throughput levels that favor broad adoption.

### SUMMARY

Aspects of the present disclosure provide a composition comprising: an avidin protein comprising at least two biotin binding sites; a first biotin moiety bound to at least one biotin binding site on the avidin protein; and a polyglycine peptide attached to the first biotin moiety through a linker, wherein the linker comprises a polyethylene glycol (PEG) moiety.

In some embodiments, the polyglycine peptide comprises at least two glycine residues (e.g., 2-10, 2-5, 3-5, 2, 3, 4, 5, 6, 7, 8, 9, or 10 glycine residues). In some embodiments, the polyglycine peptide comprises an N-terminal glycine residue having a free amino terminus. In some embodiments, the polyglycine peptide comprises a C-terminal glycine residue attached to the linker. In some embodiments, the C-terminal glycine residue is covalently attached to the PEG moiety. In some embodiments, the PEG moiety is of the formula:

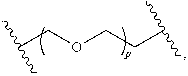

wherein p is an integer from 1-10, inclusive. In some embodiments, p is 1-5, 2-5, or 2-4. In some embodiments, p is 1. In some embodiments, p is 2. In some embodiments, p is 3. In some embodiments, p is 4. In some embodiments, p is 5.

In some embodiments, the first biotin moiety comprises a bis-biotin moiety bound to two biotin binding sites on the avidin protein. In some embodiments, the avidin protein comprises streptavidin.

In some embodiments, the avidin protein comprises at least one biotin binding site that is unbound. In some embodiments, the linker further comprises a nucleic acid. In some embodiments, the first biotin moiety is covalently attached to the nucleic acid. In some embodiments, the PEG moiety forms a linkage group between the polyglycine peptide and the nucleic acid. In some embodiments, the linker further comprises a peptide forming a linkage group between the PEG moiety and the nucleic acid. In some embodiments, the peptide has an amino acid sequence of KFFDDDGGGDD (SEQ ID NO: 1). In some embodiments, the linker-polyglycine comprises the formula of: X-PEG₄-GGG, where: X is a peptide having an amino acid sequence of KFFDDDGGGDD (SEQ ID NO: 1); PEG4 is a PEG moiety having four ethylene glycol subunits; and GGG is a polyglycine peptide having three glycine residues.

In some embodiments, the composition further comprises: a second biotin moiety bound to at least one biotin binding site on the avidin protein; and a nucleic acid attached to the second biotin moiety, wherein the nucleic acid comprises one or more luminescent labels. In some embodiments, the nucleic acid comprises at least two fluorophore dyes (e.g., 2-20, 2-10, 3-10, 5-15, 2, 3, 4, 5, 6, 7, 8, 9, or 10 fluorophore dyes). In some embodiments, each fluorophore dye is attached to a different attachment site on the nucleic acid. In some embodiments, the first biotin moiety is covalently attached to the PEG moiety. In some embodiments, the second biotin moiety comprises a bis-biotin moiety bound to two biotin binding sites on the avidin protein. In some embodiments, the avidin protein comprises four biotin binding sites, the first biotin moiety comprises a bis-biotin moiety bound to two biotin binding sites on the avidin protein, and the second biotin moiety comprises a bis-biotin moiety bound to two biotin binding sites on the avidin protein.

Aspects of the present disclosure provide a method comprising: (a) contacting a fusion polypeptide comprising a peptide barcode with any composition described herein that comprises a polyglycine peptide and an avidin protein; and (b) enzymatically conjugating the peptide barcode to the polyglycine peptide, thereby forming a conjugate comprising the peptide barcode and the avidin protein.

In some embodiments, the fusion polypeptide comprises a sortase recognition sequence fused to the peptide barcode. In some embodiments, the peptide barcode is enzymatically conjugated to the polyglycine peptide by a sortase enzyme. In some embodiments, the sortase enzyme is sortase A.

In some embodiments, the method further comprises: (c) contacting the conjugate with a biotinylated surface, thereby immobilizing the peptide barcode to the biotinylated surface through the avidin protein.

In some embodiments, the fusion polypeptide comprises a protein of interest fused to the peptide barcode. In some embodiments, the method further comprises: prior to (a), cleaving the protein of interest from the fusion polypeptide; or after (b) and prior to (c), cleaving the protein of interest from the fusion polypeptide. In some embodiments, the fusion polypeptide comprises a cleavage site between the protein of interest and the peptide barcode. In some embodiments, the cleavage site is a LysC cleavage site. In some embodiments, the cleaving comprises contacting the fusion polypeptide with endoproteinase LysC.

In some embodiments, the method further comprises: (d) sequencing the peptide barcode. In some embodiments, the sequencing comprises: contacting the peptide barcode with one or more amino acid recognition molecules; and monitoring a signal for signal pulses corresponding to binding interactions between the one or more amino acid recognition molecules and successive amino acids exposed at a terminus of the peptide barcode while the peptide barcode is being degraded.

Aspects of the present disclosure provide a method comprising: expressing a library of fusion polypeptides, each fusion polypeptide of the library comprising a protein of interest fused to a peptide barcode, wherein the peptide barcode is indicative of the protein of interest to which it is fused; immobilizing the library of fusion polypeptides to a surface; contacting the library of fusion polypeptides with a labeled binding reagent; detecting signals indicative of binding between the labeled binding reagent and the protein of interest of at least one fusion polypeptide of the library; cleaving the peptide barcode from the at least one fusion polypeptide comprising the protein of interest; contacting the peptide barcode with any composition described herein that comprises a polyglycine peptide and an avidin protein; enzymatically conjugating the peptide barcode to the polyglycine peptide, thereby forming a conjugate comprising the peptide barcode and the avidin protein; contacting the conjugate with a biotinylated surface, thereby immobilizing the peptide barcode to the biotinylated surface through the avidin protein; and sequencing the peptide barcode to identify the protein of interest of the at least one fusion polypeptide.

Aspects of the present disclosure provide a method of identifying a target analyte in a region of a sample, the method comprising: contacting a sample with an affinity reagent conjugated to a peptide barcode indicative of a target analyte to which the affinity reagent binds; releasing the peptide barcode from the affinity reagent in a first region of the sample; contacting the peptide barcode with any composition described herein that comprises a polyglycine peptide and an avidin protein; enzymatically conjugating the peptide barcode to the polyglycine peptide, thereby forming a conjugate comprising the peptide barcode and the avidin protein; contacting the conjugate with a biotinylated surface, thereby immobilizing the peptide barcode to the biotinylated surface through the avidin protein; and sequencing the peptide barcode to identify the target analyte in the first region of the sample.

Aspects of the present disclosure relate to a method of identifying target analytes in a sample, the method comprising: (a) contacting a sample with a plurality of affinity reagents conjugated to different peptide barcodes indicative of different target analytes; (b) releasing one or more peptide barcodes from one or more respective affinity reagents in a first region of the sample; (c) releasing one or more peptide barcodes from one or more respective affinity reagents in a second region of the sample; (d) immobilizing the one or more peptide barcodes released from each of the first and second regions of the sample to a surface, wherein the immobilizing comprises: contacting a peptide barcode with any composition described herein that comprises a polyglycine peptide and an avidin protein; enzymatically conjugating the peptide barcode to the polyglycine peptide, thereby forming a conjugate comprising the peptide barcode and the avidin protein; contacting the conjugate with a biotinylated surface, thereby immobilizing the peptide barcode to the biotinylated surface through the avidin protein; and (e) sequencing the one or more peptide barcodes released from each of the first and second regions of the sample.

Aspects of the present disclosure provide a method of identifying a therapeutic agent in a region of a sample, the method comprising: contacting a sample with a therapeutic agent conjugated to a peptide barcode indicative of the therapeutic agent; releasing the peptide barcode from the therapeutic agent in a first region of the sample; contacting the peptide barcode with any composition described herein that comprises a polyglycine peptide and an avidin protein; enzymatically conjugating the peptide barcode to the polyglycine peptide, thereby forming a conjugate comprising the peptide barcode and the avidin protein; contacting the conjugate with a biotinylated surface, thereby immobilizing the peptide barcode to the biotinylated surface through the avidin protein; and sequencing the peptide barcode to identify the therapeutic agent in the first region of the sample.

Aspects of the present disclosure provide a method of identifying delivery to a target cell, the method comprising: contacting a cell with a delivery agent that comprises a polynucleotide, wherein the polynucleotide encodes a protein of interest fused to a peptide barcode; contacting the cell with a cleaving agent configured to cleave the peptide barcode from the protein of interest; contacting the peptide barcode with any composition described herein that comprises a polyglycine peptide and an avidin protein; enzymatically conjugating the peptide barcode to the polyglycine peptide, thereby forming a conjugate comprising the peptide barcode and the avidin protein; contacting the conjugate with a biotinylated surface, thereby immobilizing the peptide barcode to the biotinylated surface through the avidin protein; and detecting the peptide barcode to identify uptake of the polynucleotide by the cell.

Aspects of the present disclosure provide a method of evaluating nanoparticle delivery to a target cell, the method comprising: contacting a cell with a plurality of nanoparticles having different lipid compositions, wherein each nanoparticle encapsulates a polynucleotide, wherein the polynucleotide encodes a protein of interest fused to a peptide barcode indicative of lipid composition of the nanoparticle; contacting the cell with a cleaving agent configured to cleave the peptide barcode from the protein of interest; contacting the peptide barcode with any composition described herein that comprises a polyglycine peptide and an avidin protein; enzymatically conjugating the peptide barcode to the polyglycine peptide, thereby forming a conjugate comprising the peptide barcode and the avidin protein; contacting the conjugate with a biotinylated surface, thereby immobilizing the peptide barcode to the biotinylated surface through the avidin protein; and sequencing the peptide barcode to identify the lipid composition of the nanoparticle.

Aspects of the present disclosure provide a method of evaluating protein production, the method comprising: contacting a cell with an mRNA molecule encoding a protein of interest and a peptide barcode; contacting the cell with a cleaving agent configured to cleave the peptide barcode from the protein of interest; contacting the peptide barcode with any composition described herein that comprises a polyglycine peptide and an avidin protein; enzymatically conjugating the peptide barcode to the polyglycine peptide, thereby forming a conjugate comprising the peptide barcode and the avidin protein; contacting the conjugate with a biotinylated surface, thereby immobilizing the peptide barcode to the biotinylated surface through the avidin protein; and detecting the peptide barcode to identify production of the protein of interest.

Aspects of the present disclosure provide a method comprising: (a) contacting a fusion polypeptide comprising a protein of interest with any composition described herein that comprises a polyglycine peptide and one or more luminescent labels; and (b) enzymatically conjugating the protein of interest to the polyglycine peptide, thereby forming a conjugate comprising the protein of interest and the one or more luminescent labels.

In some embodiments, the fusion polypeptide comprises a sortase recognition sequence fused to the protein of interest. In some embodiments, the protein of interest is enzymatically conjugated to the polyglycine peptide by a sortase enzyme. In some embodiments, the sortase enzyme is sortase A.

In some embodiments, the method further comprises: (c) contacting the conjugate with a surface-immobilized compound; and (d) monitoring a luminescent signal for signal pulses corresponding to binding interactions between the protein of interest and the surface-immobilized compound. In some embodiments, the surface-immobilized compound is a surface-immobilized polypeptide. In some embodiments, the surface-immobilized compound is a surface-immobilized small molecule.

Aspects of the present disclosure provide a method of screening for modulators of a protein of interest, the method comprising: (a) contacting a fusion polypeptide comprising a protein of interest with any composition described herein that comprises a polyglycine peptide and one or more luminescent labels; (b) enzymatically conjugating the protein of interest to the polyglycine peptide, thereby forming a conjugate comprising the protein of interest and the one or more luminescent labels; (c) contacting the conjugate with a substrate comprising a library of different compounds immobilized to a surface of the substrate; and (d) monitoring a luminescent signal for signal pulses corresponding to binding interactions between the protein of interest and at least one compound of the library.

In some embodiments, the library of different compounds comprises at least two, at least five, at least 10, at least 20, at least 30, at least 50, at least 75, at least 100, at least 200, at least 250, at least 500, or at least 1000 different compounds. In some embodiments, each of the different compounds is independently a polypeptide or a small molecule.

Aspects of the present disclosure provide a method of polypeptide sequencing, the method comprising: (a) contacting a fusion polypeptide comprising a protein of interest with any composition described herein that comprises a polyglycine peptide and one or more luminescent labels; (b) enzymatically conjugating the protein of interest to the polyglycine peptide, thereby forming a conjugate comprising the protein of interest and the one or more luminescent labels; (c) contacting the conjugate with a polypeptide; and (d) monitoring a luminescent signal for signal pulses corresponding to binding interactions between the protein of interest and successive amino acids exposed at a terminus of the polypeptide while the polypeptide is being degraded.

In some embodiments, (c) comprises contacting the polypeptide with the conjugate and one or more luminescently labeled amino acid recognition molecules. In some embodiments, the method is performed in a reaction mixture comprising one or more cleaving reagents that degrade the polypeptide. In some embodiments, the one or more cleaving reagents comprise one or more aminopeptidases. In some embodiments, the protein of interest is an affinity reagent. In some embodiments, the affinity reagent is an antibody or an adaptor protein.

Aspects of the present disclosure relate to a fusion polypeptide, comprising a protein of interest, an affinity tag, a linker, a cleavage site, a barcode, and a sortase tag (e.g., a sortase recognition sequence).

In some embodiments, the affinity tag is a FLAG-tag, an HA-tag, a myc-tag, or a V5 tag.

In some embodiments, the linker is a glycine-serine (GS) linker.

In some embodiments, the cleavage site is a LysC cleavage site.

In some embodiments, the barcode is a peptide barcode.

In some embodiments, the sortase tag comprises the amino acid sequence LPXTG (SEQ ID NO: 2), where X is any amino acid. In some embodiments, the sortase tag comprises the amino acid sequence LPETG (SEQ ID NO: 3).

In some embodiments, the fusion polypeptide further comprises a His-tag. In some embodiments, the His-tag is a 6x-His-tag (SEQ ID NO: 4).

Aspects of the present disclosure relate to a method of peptide sequencing, comprising: obtaining any fusion polypeptide described herein; enriching the fusion polypeptide by affinity capture; contacting the fusion polypeptide with a sortase; contacting the fusion polypeptide with an endoproteinase to isolate the barcode; and sequencing the barcode.

In some embodiments, the affinity capture comprises contacting the fusion polypeptide with a molecule that binds to a FLAG-tag, an HA-tag, a myc-tag, or a V5 tag.

In some embodiments, the contacting with the sortase comprises contacting the fusion polypeptide with a sortase and a composition described herein that comprises a polyglycine peptide and an avidin protein. In some embodiments, the polyglycine peptide is enzymatically conjugated to the sortase tag by the sortase to form a conjugate comprising the peptide barcode and the avidin protein. In some embodiments, the method further comprises, prior to the sequencing, contacting the conjugate with a biotinylated surface, thereby immobilizing the peptide barcode to the biotinylated surface through the avidin protein.

In some embodiments, the sortase is sortase A.

In some embodiments, the endoproteinase is LysC.

In some embodiments, the barcode is a peptide barcode.

In some embodiments, the sequence of the barcode is indicative of the identity of the protein of interest.

Aspects of the present disclosure relate to a method of tracking a protein of interest in a subject, the method comprising: administering to the subject a messenger RNA (mRNA) molecule encoding any fusion protein described herein; extracting from the subject a tissue of interest; and executing any method described herein.

In some embodiments, the administering comprises administering a viral particles of lipid nanoparticle comprising the mRNA.

In some embodiments, the tissue of interest is spleen tissue, liver tissue, kidney tissue, brain tissue, lung tissue, cardiac tissue, or blood.

Aspects of the present disclosure relate to a library of fusion polypeptides, comprising a first subset of fusion polypeptides and a second subset of fusion polypeptides, wherein each fusion polypeptide within the first subset of fusion polypeptides comprises a protein of interest, a first affinity tag, a linker, a cleavage site, a barcode, and a sortase tag, and wherein each fusion polypeptide within the second subset of fusion polypeptides comprises a protein of interest, a second affinity tag, a linker, a cleavage site, a barcode, and a sortase tag.

In some embodiments, the first affinity tag is a FLAG-tag, an HA-tag, a myc-tag, or a V5 tag.

In some embodiments, the second affinity tag is a FLAG-tag, an HA-tag, a myc-tag, or a V5 tag.

In some embodiments, the library further comprises a third subset of fusion polypeptides, wherein each fusion polypeptide within the third subset of fusion polypeptides comprises a protein of interest, a third affinity tag, a linker, a cleavage site, a barcode, and a sortase tag.

In some embodiments, the third affinity tag is a FLAG-tag, an HA-tag, a myc-tag, or a V5 tag.

In some embodiments, the library further comprises a fourth subset of fusion polypeptides, wherein each fusion polypeptide within the fourth subset of fusion polypeptides comprises a protein of interest, a fourth affinity tag, a linker, a cleavage site, a barcode, and a sortase tag.

In some embodiments, the fourth affinity tag is a FLAG-tag, an HA-tag, a myc-tag, or a V5 tag.

In some embodiments, the library comprises five or more (e.g., 5-50, 10-100, 15-30, 20-200) subsets of fusion polypeptides.

In some embodiments, each affinity tag is different.

In some embodiments, the linker is a glycine-serine (GS) linker.

In some embodiments, the cleavage site is a LysC cleavage site.

In some embodiments, the barcode is a peptide barcode.

In some embodiments, the sortase tag comprises the amino acid sequence LPXTG (SEQ ID NO: 2), where X is any amino acid. In some embodiments, the sortase tag comprises the amino acid sequence LPETG (SEQ ID NO: 3).

In some embodiments, each fusion polypeptide further comprises a His-tag.

In some embodiments, the His-tag is a 6x-His-tag (SEQ ID NO: 4).

Aspects of the present disclosure relate to a method of peptide sequencing, the method comprising: obtaining any library described herein; contacting the first subset of fusion polypeptides with a molecule that binds to the first affinity tag; contacting the first subset of fusion polypeptides with a sortase; contacting the first subset of fusion polypeptides with an endoproteinase to isolate the barcode of the first subset of fusion polypeptides; sequencing the barcode of the first subset of fusion polypeptides; contacting the second subset of fusion polypeptides with a molecule that binds to the second affinity tag; contacting the second subset of fusion polypeptides with a sortase; contacting the second subset of fusion polypeptides with an endoproteinase to isolate the barcode of the second subset of fusion polypeptides; and sequencing the barcode of the second subset of fusion polypeptides.

In some embodiments, the method further comprises contacting the third subset of fusion polypeptides with a molecule that binds to the third affinity tag; contacting the third subset of fusion polypeptides with a sortase; contacting the third subset of fusion polypeptides with an endoproteinase to isolate the barcode of the third subset of fusion polypeptides; and sequencing the barcode of the third subset of fusion polypeptides.

In some embodiments, the method further comprises contacting the fourth subset of fusion polypeptides with a molecule that binds to the fourth affinity tag; contacting the fourth subset of fusion polypeptides with a sortase; contacting the fourth subset of fusion polypeptides with an endoproteinase to isolate the barcode of the fourth subset of fusion polypeptides; and sequencing the barcode of the fourth subset of fusion polypeptides.

In some embodiments, the barcodes within the first subset of fusion polypeptides, the second subset of fusion polypeptides, the third subset of fusion polypeptides, and the fourth subset of fusion polypeptides are each different within subsets of fusion polypeptides but the same across subsets of fusion polypeptides.

Aspects of the present disclosure relate to a kit, comprising any composition described herein, any fusion polypeptide described herein, or any library described herein, and materials and/or reagents for executing any method described herein.

In some embodiments, the kit further comprises instructions for executing any method described herein.

The details of certain embodiments of the disclosure are set forth in the Detailed Description Other features, objects, and advantages of the disclosure will be apparent from the Examples, Drawings, and Claims.

### BRIEF DESCRIPTION OF THE DRAWINGS

The accompanying Drawings, which constitute a part of this specification, illustrate several embodiments of the disclosure and together with the accompanying description, serve to explain the principles of the disclosure.

FIG. 1A shows an example workflow for functionalizing a barcode or barcoded molecule for surface immobilization by ligation of a streptavidin conjugate in accordance with embodiments of the disclosure.

FIG. 1B shows an example workflow for functionalizing a protein of interest (POI), such as an affinity reagent, for evaluating binding by ligation of a dye-labeled streptavidin conjugate in accordance with embodiments of the disclosure.

FIG. 1C shows an example overview of real-time dynamic protein sequencing. Protein samples are digested into peptide fragments, immobilized in nanoscale reaction chambers, and incubated with a mixture of freely diffusing N-terminal amino acid (NAA) recognizers and aminopeptidases that carry out the sequencing process. The labeled recognizers bind on and off to the peptide when one of their cognate NAAs is exposed at the N-terminus, thereby producing characteristic pulsing patterns. The NAA is cleaved by an aminopeptidase, exposing the next amino acid for recognition. The temporal order of NAA recognition and the kinetics of binding enable peptide identification and are sensitive to features that modulate binding kinetics, such as post-translational modifications (PTMs).

FIGS. 2A-2B show example workflows for functionalizing a barcode of a fusion polypeptide for surface immobilization by ligation of a polyglycine-streptavidin conjugate (“polyG-linker”). FIG. 2C shows example configurations of barcodes attached to N-terminal or C-terminal positions relative to a POI. FIG. 2D shows an example construct of a polyG-linker. FIG. 2E shows an example construct of a barcode functionalized with a polyG-linker.

FIG. 3 shows an example workflow of barcode functionalization.

FIGS. 4A-4C show an overview of peptide sequencing, a protein barcoding construct design, and a barcoding workflow. FIG. 4A shows an overview of the sequencing instrument and the principle of Next-Generation Protein Sequencing (NGPS). After single peptides are bound to the semiconductor chip, fluorescently tagged amino acid recognizers (six recognizers for 13 amino acids) bind each N-terminal amino acid. After aminopeptidase cleavage, the next amino acid is bound. FIG. 4B shows a barcoding construct design including the protein of interest, followed by an affinity tag for purification, a short linker, a LysC cleavage site, the peptide barcode, a sortase tag for attachment of a covalent linker for sequencing, and an optional His-tag for purification. FIG. 4C shows barcoded protein enrichment and barcode sequencing workflow showing the steps going from cell lysate to sequencing.

FIGS. 5A-5B shows the computational design of protein barcodes for NGPS. FIG. 5A shows a barcode design workflow selects optimal barcode designs by taking into account protein sequencing kinetics and Levenshtein edit distance to produce barcodes with optimal properties for multiplexing. FIG. 5B is a schematic of the computational selection and refinement of barcodes to the eight used in this study.

FIGS. 6A-6D shows normalization and reproducibility in 8-barcode mixtures. FIG. 6A is a schematic of normalization workflow showing the strategy for converting raw alignments to normalized alignments, enabling calculation of inferred relative barcode fractions. FIG. 6B shows that alignments were normalized and relative fraction recovered for eight runs containing 1:1 eight-barcode mixtures. FIG. 6C shows False Discovery Rate (FDR) for normalized alignments across all eight runs; dotted line indicates 10% FDR. FIG. 6D shows a performance summary of recovered inferred fractions for all eight runs plotted individually. MAPE=mean absolute percent error.

FIGS. 7A-7C shows the limit of detection (LOD) for all eight tested barcodes. FIG. 7A shows that alignments were normalized for a 10-fold dynamic range titration; in this example, the least abundant barcode (BC032) was positively identified at ~400 fmol input. FIG. 7B shows inferred fraction vs. true fraction for the data in FIG. 7A. FIG. 7C shows LOD values in an eight-plex mixture for each barcode tested in this study.

FIGS. 8A-8D shows a ten-fold dynamic range of eight barcodes. FIG. 8A shows that alignments were normalized for a ten-fold dynamic range titration at the following levels: 1× (BC051), 0.75× (BC028, and BC096), 0.5× (BC075, and BC079), 0.25× (BC032, and BC049), and 0.1× (BC067). FIG. 8B shows inferred fraction vs. true fraction for the data in FIG. 8A. FIG. 8C shows normalized alignments for eight runs at the following titration levels: 1× (BC028), 0.75× (BC032, and BC049), 0.5× (BC051, and BC067), 0.25× (BC075, and BC079), and 0.1× (BC096). FIG. 8D shows FDR for the same runs shown in FIG. 8C; dotted line indicates 10% FDR cutoff.

FIGS. 9A-9D shows an equimolar mix of five barcoded proteins. FIG. 9A shows a summary and characteristics of the five proteins tested in this study. MW=Molecular Weight. FIG. 9B shows normalized alignments recovered across eight runs containing the five proteins mixed at equimolar concentrations. FIG. 9C shows FDR across the eight runs shown in FIG. 9B; dotted line indicates 10% FDR cutoff. FIG. 9D shows a performance summary of recovered inferred fractions for all eight runs plotted individually.

FIG. 10 shows an example construct of barcoded SARS-COV2-S1-RBD (319-541).

FIG. 11 shows experimental results demonstrating significantly improved sensitivity with barcodes functionalized with polyG-linkers described herein.

FIG. 12 shows experimental results demonstrating comparable activity of sortase A5 and sortase A Pentamutant (BPS Biosciences).

FIG. 13 shows experimental results for SARS-COV2-RBD and p53 proteins prepared and analyzed according to the workflow of FIG. 3.

FIG. 14 shows experimental results for three model synthetic peptides analyzed in a 3-barcode mixture (25 pmol total with each barcode at 8.3 pmol, and 8.3 pmol total with each barcode at ~2.8 pmol).

FIG. 15 shows a barcode workflow that is used, in some embodiments, with the barcode plexity expansion construct shown in FIG. 16 and described elsewhere herein.

FIG. 16 is a schematic showing a protein construct useful for increasing barcode plexity by using various affinity tags with the same 24 peptide barcodes.

FIG. 17 shows conventional approaches for therapeutic peptide screening.

FIG. 18 shows reagents that can be used in accordance with dye-labeling methods described herein.

FIG. 19 shows an example workflow for a dye-labeling method described herein.

FIG. 20 shows an arginine recognizer as a model system for dye-labeling according to the workflow of FIG. 19.

FIG. 21 shows an experimental workflow evaluating dye-labeling according to the workflow of FIG. 19.

FIG. 22 shows an exemplary output produced by an arginine recognizer that was dye-labeled according to the workflow of FIG. 19.

FIG. 23 shows an example workflow for the use of peptide barcodes to monitor protein expression.

FIGS. 24A-24B show example constructs of dye-labeled polyG-linkers for use in labeling a POI according to the example workflow of FIG. 1B.

FIG. 25 shows an example overview of dye-labeling POIs for detecting binding of POIs to peptides prior to peptide sequencing.

FIG. 26 shows an example workflow for dye-labeling a POI and detecting binding of the labeled POI to peptides prior to peptide sequencing.

FIG. 27 shows an example construct of a model protein (RBD) for dye-labeling as described herein.

FIG. 28 shows an example construct of a model protein (p53) for dye-labeling as described herein.

FIG. 29 shows experimental results demonstrating detection of binding between a target molecule and an affinity reagent (PS1220) labeled with a polyG-linker described herein.

FIG. 30 shows experimental results demonstrating purity of the polyG-linker dye scaffold.

FIG. 31 shows an example construct of a recognizer-AviTag-biotinylation-scaffold for dye-labeling with polyG-linkers described herein.

FIGS. 32A-32I show experimental results for proteins that were dye-labeled using polyG-linkers described herein.

FIG. 33 shows the BCL-2 regulated apoptotic pathway (left) and a structural model of BCL-2 bound with BID-BH3 peptide.

FIG. 34 shows an experimental output of using a dye labeling method of the disclosure to identify BLC2 labeled protein bound to BID-BH3 peptide (left) and PS1220 labeled protein bound to control R-start peptide (right).

FIGS. 35A-35C show recovery of relative abundance in an equimolar mix of 24 barcoded proteins. FIG. 35A shows raw alignments. FIG. 35B shows normalized alignments.

FIG. 35C shows inferred fraction. The barcode sequences are shown in Table 2.

FIG. 36 shows 24-plex barcodes across a 240-fold dynamic range.

### DETAILED DESCRIPTION

Protein barcoding has emerged as a powerful tool for the multiplexed identification and characterization of proteins, providing a mechanism for precise tracking of protein affinity, location, and expression. Accordingly, aspects of the present disclosure relate to the development of a protein barcoding workflow for use with single-molecule analytical instruments. Aspects of the present disclosure are based in part on the experimental validation of peptide barcodes, each designed to minimize detection bias and maximize sensitivity across various experimental conditions. The inventors of the present disclosure also optimized the design of expression constructs to decrease both the hands-on time and input requirements of the workflow. In this workflow, affinity-tagged proteins are expressed with unique peptide barcodes. Following experimental selection or treatments, the proteins are purified, and the peptide barcodes are cleaved and sequenced on a sequencing instrument. The present disclosure also demonstrates that barcodes at considerably low concentrations of sample input can be detected within a multiplex barcode mixture (e.g., approximately 100 fmol of sample input in a 24-plex mixture). The present disclosure also shows the capacity of this barcoding approach to achieve a ten-fold dynamic range, underscoring its sensitivity in recovering variants with low abundance.

Aspects of the disclosure relate to compositions and methods for functionalizing a polypeptide for single-molecule analysis. In some embodiments, the polypeptide is a fusion polypeptide comprising a peptide barcode. In some embodiments, the polypeptide is a fusion polypeptide comprising a protein of interest.

In some aspects, the disclosure provides compositions that can be enzymatically conjugated to a fusion polypeptide as described herein. In some embodiments, the composition comprises: an avidin protein comprising at least two biotin binding sites; a first biotin moiety bound to at least one biotin binding site on the avidin protein; and a polyglycine peptide attached to the first biotin moiety through a linker, where the linker comprises a polyethylene glycol (PEG) moiety.

In some embodiments, the polyglycine peptide comprises at least two glycine residues. In some embodiments, the polyglycine peptide comprises 2-10, 2-5, 3-5, 2, 3, 4, 5, 6, 7, 8, 9, or 10 glycine residues. In some embodiments, the polyglycine peptide comprises an N-terminal glycine residue having a free amino terminus. In some embodiments, the polyglycine peptide comprises a C-terminal glycine residue attached to the linker. In some embodiments, the C-terminal glycine residue is covalently attached to the PEG moiety.

In some embodiments, the PEG moiety of a linker described herein is of the formula:

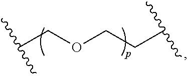

where p is an integer from 1-10, inclusive. In some embodiments, p is an integer from 2-8, inclusive. In some embodiments, p is an integer from 1-5, inclusive. In some embodiments, p is an integer from 2-5, inclusive. In some embodiments, p is 1. In some embodiments, p is 2. In some embodiments, p is 3. In some embodiments, p is 4. In some embodiments, p is 5. In some embodiments, p is 6.

In some embodiments, the polyglycine peptide is attached to the avidin protein through a first biotin moiety bound to at least one biotin binding site on the avidin protein. In some embodiments, the first biotin moiety comprises a bis-biotin moiety bound to two biotin binding sites on the avidin protein. In some embodiments, the avidin protein comprises four biotin binding sites. In some embodiments, the avidin protein comprises streptavidin.

In some aspects, the disclosure provides methods of enzymatically conjugating a composition comprising a polyglycine peptide as described herein to a fusion polypeptide. In some embodiments, the composition is enzymatically conjugated to the polyglycine peptide by a sortase enzyme (e.g., sortase A). In some embodiments, the fusion polypeptide comprises a sortase recognition sequence. The terms “sortase tag” and “sortase recognition sequence” are used interchangeably herein. In some embodiments, the sortase enzyme conjugates the polyglycine peptide of the composition to the sortase recognition sequence of the fusion polypeptide. In some embodiments, the sortase recognition sequence comprises the amino acid sequence LPXTG (SEQ ID NO: 2), where X is any amino acid. In some embodiments, the sortase recognition sequence comprises an amino acid sequence of LPETG (SEQ ID NO: 3). In some embodiments, the sortase recognition sequence comprises an amino acid sequence of LPETGG (SEQ ID NO: 5). Other suitable sortase recognition sequences are known in the art.

Functionalization with Surface Immobilization Linker

In some aspects, the disclosure provides compositions that can be enzymatically conjugated to a fusion polypeptide comprising a peptide barcode for surface immobilization. In some embodiments, the composition comprises: an avidin protein comprising at least two biotin binding sites; a first biotin moiety bound to at least one biotin binding site on the avidin protein; and a polyglycine peptide attached to the first biotin moiety through a linker, where the linker comprises a polyethylene glycol (PEG) moiety. In some embodiments, the avidin protein comprises at least one biotin binding site that is unbound. In this way, conjugation of the composition to the fusion polypeptide provides at least one unbound biotin binding site available for immobilization of the peptide barcode to a biotinylated surface.

In some embodiments, the linker further comprises a nucleic acid. In some embodiments, the nucleic acid comprises DNA, RNA, or a combination thereof. In some embodiments, the nucleic acid is a double-stranded nucleic acid. In some embodiments, the nucleic acid is a single-stranded nucleic acid. In some embodiments, the nucleic acid is between about 5 and about 100 bases or base pairs in length. In some embodiments, the nucleic acid is between about 5 and about 50 bases or base pairs in length. In some embodiments, the nucleic acid is between about 10 and about 75 bases or base pairs in length. In some embodiments, the nucleic acid is between about 20 and about 60 bases or base pairs in length.

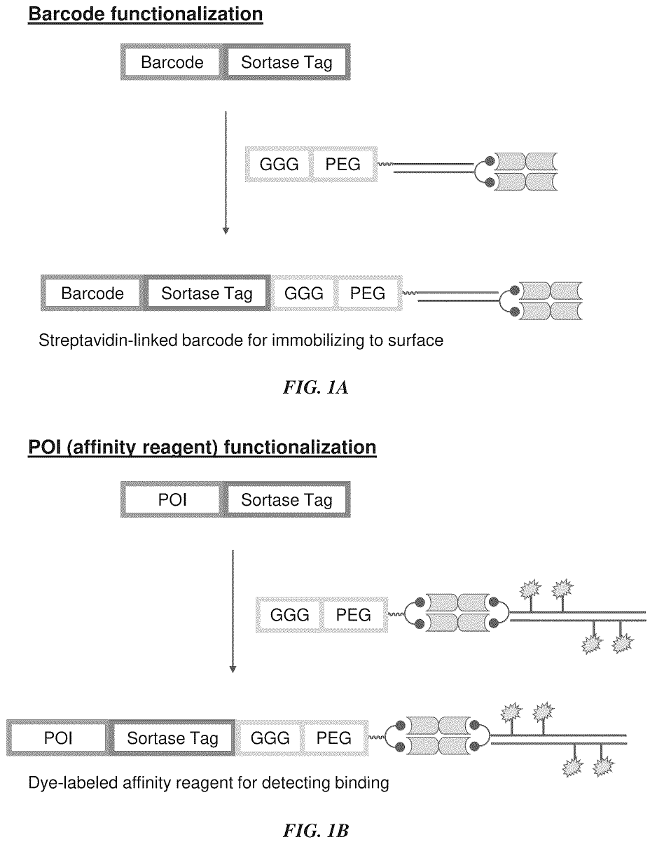

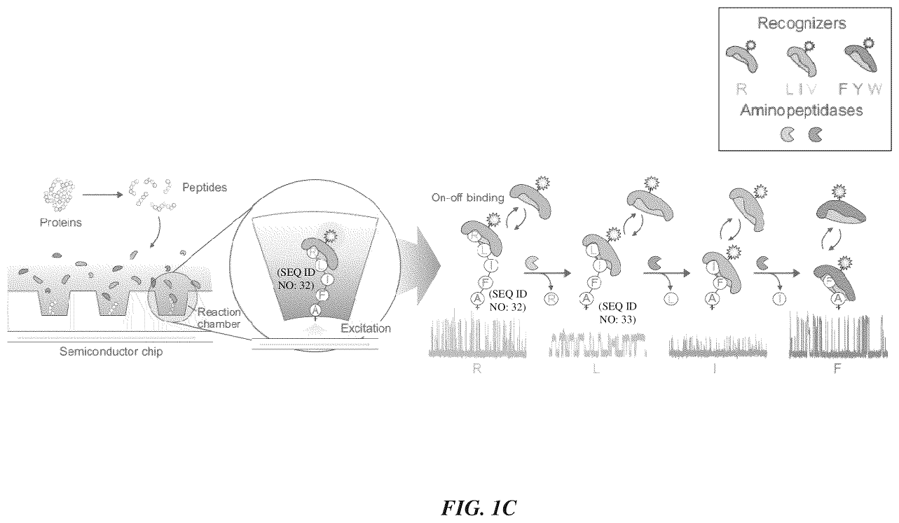

In some embodiments, the first biotin moiety is covalently attached to the nucleic acid. In some embodiments, the PEG moiety forms a linkage group between the polyglycine peptide and the nucleic acid. See, e.g., an example polyglycine-streptavidin composition used for conjugation in the workflow depicted in FIG. 1A.

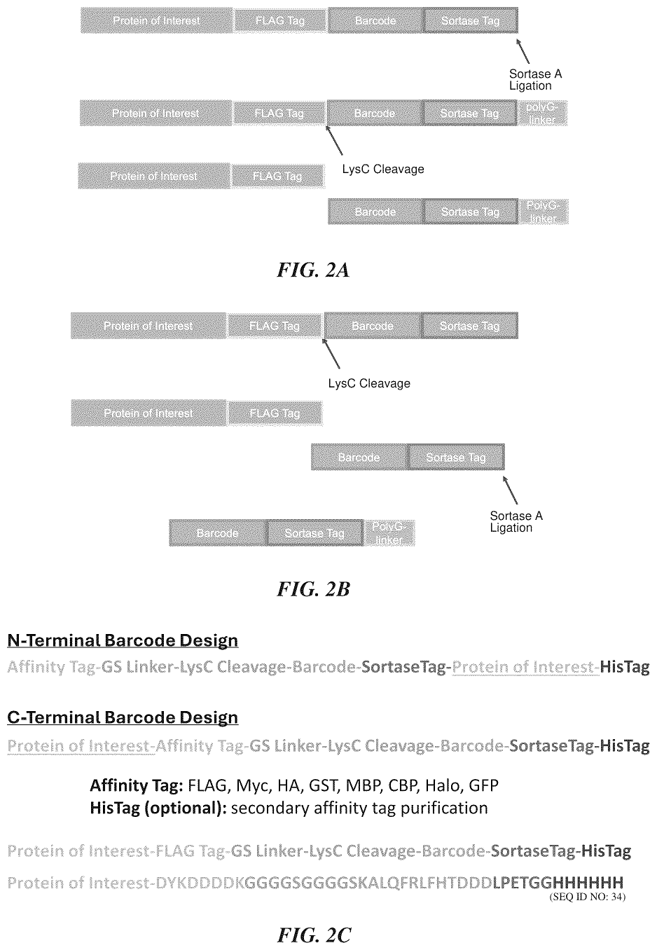

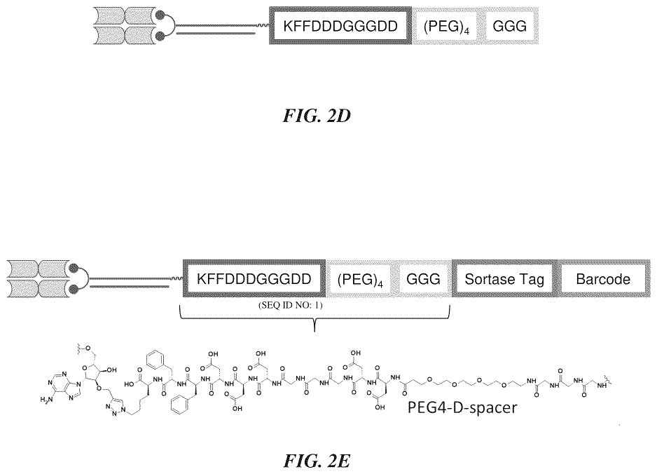

In some embodiments, the linker further comprises a peptide forming a linkage group between the PEG moiety and the nucleic acid. In some embodiments, the peptide is at least 5 amino acids in length. In some embodiments, the peptide is between about 3 and about 20 amino acids in length. In some embodiments, the peptide is between about 5 and about 15 amino acids in length. In some embodiments, the peptide has an amino acid sequence of KFFDDDGGGDD (SEQ ID NO: 1). See, e.g., an example polyglycine-peptide composition depicted in FIG. 2D. In some embodiments, the linker-polyglycine comprises the formula of: X-PEG4-GGG, where: X is a peptide having an amino acid sequence of KFFDDDGGGDD (SEQ ID NO: 1); PEG4 is a PEG moiety having four ethylene glycol subunits; and GGG is a polyglycine peptide having three glycine residues.

In some aspects, the disclosure provides methods comprising: (a) contacting a fusion polypeptide comprising a peptide barcode with a composition comprising a polyglycine peptide and an avidin protein having at least one unbound biotin binding site as described herein; and (b) enzymatically conjugating the peptide barcode to the polyglycine peptide, thereby forming a conjugate comprising the peptide barcode and the avidin protein. In some embodiments, the method further comprises: (c) contacting the conjugate with a biotinylated surface, thereby immobilizing the peptide barcode to the biotinylated surface through the avidin protein.

In some embodiments, the fusion polypeptide comprises a protein of interest fused to the peptide barcode. In some embodiments, the method further comprises, prior to (a), cleaving the protein of interest from the fusion polypeptide. In some embodiments, the method further comprises, after (b) and prior to (c), cleaving the protein of interest from the fusion polypeptide. In some embodiments, the fusion polypeptide comprises a cleavage site between the protein of interest and the peptide barcode. In some embodiments, the cleavage site is a LysC cleavage site. In some embodiments, the cleaving comprises contacting the fusion polypeptide with endoproteinase LysC.

In some embodiments, the method further comprises: (d) sequencing the peptide barcode. In some embodiments, the sequencing is as described herein. In some embodiments, the sequencing comprises: contacting the peptide barcode with one or more amino acid recognition molecules; and monitoring a signal for signal pulses corresponding to binding interactions between the one or more amino acid recognition molecules and successive amino acids exposed at a terminus of the peptide barcode while the peptide barcode is being degraded.

FIGS. 2A-2B generically depict example workflows for functionalizing a fusion polypeptide comprising a protein of interest (POI) and a peptide barcode. As shown in FIG. 2A, in some embodiments, the POI-barcode fusion polypeptide is enzymatically conjugated with a composition described herein (“polyG-linker”), and the functionalized fusion polypeptide is subjected to LysC cleavage to remove the POI. As shown in FIG. 2B, in some embodiments, the POI-barcode fusion polypeptide is subjected to LysC cleavage to remove the POI, and the peptide barcode is enzymatically conjugated with a composition described herein (“polyG-linker”). In either approach, the resulting product is a functionalized peptide barcode that can be immobilized to a surface through the polyG-linker for single molecule analysis.

FIG. 2C shows example configurations of fusion polypeptides comprising a protein of interest and a peptide barcode. As shown, in some embodiments, the peptide barcode can be N- or C-terminal to the protein of interest in the fusion polypeptide. In some embodiments, the fusion polypeptide comprises an affinity tag. In some embodiments, the affinity tag is a FLAG tag. Other examples of such affinity tags include, without limitation, Myc, HA, GST, MBP, CBP, Halo, V5, and GFP. In some embodiments, the fusion polypeptide comprises a secondary affinity tag, such as a C-terminal histidine tag (e.g., a hexa-histidine tag or 6× His (SEQ ID NO: 4)).

Aspects of the present disclosure relate to a fusion polypeptide comprising a protein of interest (described elsewhere herein), an affinity tag (described elsewhere herein), a linker, a cleavage site (described elsewhere herein), a barcode (described elsewhere herein), and a sortase tag (described elsewhere herein). In some embodiments, a linker is a glycine-serine linker (GS linker). In some embodiments, a linker comprises the amino acid sequence GGGGSGGGGS (SEQ ID NO: 6).

Aspects of the present disclosure relate to a library of fusion polypeptides, comprising a first subset of fusion polypeptides and a second subset of fusion polypeptides, wherein each fusion polypeptide within the first subset of fusion polypeptides comprises a protein of interest, a first affinity tag, a linker, a cleavage site, a barcode, and a sortase tag, and wherein each fusion polypeptide within the second subset of fusion polypeptides comprises a protein of interest, a second affinity tag, a linker, a cleavage site, a barcode, and a sortase tag.

In some embodiments, the first affinity tag is a FLAG-tag, an HA-tag, a myc-tag, or a V5 tag.

In some embodiments, the second affinity tag is a FLAG-tag, an HA-tag, a myc-tag, or a V5 tag.

In some embodiments, the library further comprises a third subset of fusion polypeptides, wherein each fusion polypeptide within the third subset of fusion polypeptides comprises a protein of interest, a third affinity tag, a linker, a cleavage site, a barcode, and a sortase tag.

In some embodiments, the third affinity tag is a FLAG-tag, an HA-tag, a myc-tag, or a V5 tag.

In some embodiments, the library further comprises a fourth subset of fusion polypeptides, wherein each fusion polypeptide within the fourth subset of fusion polypeptides comprises a protein of interest, a fourth affinity tag, a linker, a cleavage site, a barcode, and a sortase tag.

In some embodiments, the fourth affinity tag is a FLAG-tag, an HA-tag, a myc-tag, or a V5 tag.

In some embodiments, each affinity tag is different.

In some embodiments, each subset of fusion polypeptides within a library comprises the same peptide barcodes, wherein each peptide barcode within a subset of fusion polypeptides is different but the same peptide barcodes are used across subsets. The inventors of the present disclosure made the surprising discovery that barcode plexity can be increased by using subsets of fusion polypeptides each subset comprising different affinity tags. In some embodiments, a first subset of fusion polypeptides comprises fusion polypeptides comprising the same affinity tag but different peptide barcodes. In some embodiments, a second subset of fusion polypeptides comprises fusion polypeptides comprising the same affinity tag but different peptide barcodes. In some embodiments, a third subset of fusion polypeptides comprises fusion polypeptides comprising the same affinity tag but different peptide barcodes. In some embodiments, a fourth subset of fusion polypeptides comprises fusion polypeptides comprising the same affinity tag but different peptide barcodes. In some embodiments, the same set of peptide barcodes are used in each of the four subsets of fusion polypeptides. In some embodiments, different affinity tags are used across subsets of fusion polypeptides. For example, in some embodiments, each fusion polypeptide within a first subset of fusion polypeptides comprises a first affinity tag, each fusion polypeptide within a second subset of fusion polypeptides comprises a second affinity tag, each fusion polypeptide within a third subset of fusion polypeptides comprises a third affinity tag, and each fusion polypeptide within a fourth subset of fusion polypeptides comprises a fourth affinity tag. In some embodiments, the first, second, third, and fourth affinity tags are each different. In some embodiments, each fusion polypeptide within a first subset of fusion polypeptides comprises a unique peptide barcode. In some embodiments, each fusion polypeptide within a second subset of fusion polypeptides comprises a unique peptide barcode. In some embodiments, each fusion polypeptide within a third subset of fusion polypeptides comprises a unique peptide barcode. In some embodiments, each fusion polypeptide within a fourth subset of fusion polypeptides comprises a unique peptide barcode. In some embodiments, the unique peptide barcodes across each subset are the same set of unique peptide barcodes.

A person having ordinary skill in the art will understand that many different affinity tags known in the art beyond those described herein can be used in the library described herein. A person having ordinary skill in the art will also understand that any number of subsets of fusion polypeptides having affinity tags, and not only the specific subsets described herein, can be used in the library described herein. A person having ordinary skill in the art will also understand that a set of any number of unique barcode sequences, and not only the specific barcode sequences described herein, can be used in the library described herein.

### Methods of Using Barcoded Molecules

In some aspects, the peptide barcode immobilization strategies described herein can be used in a method for identifying one or more proteins of interest to which a labeled binding reagent binds. In some embodiments, the method comprises: (a) expressing a library of fusion polypeptides, each fusion polypeptide of the library comprising a protein of interest fused to a peptide barcode, wherein the peptide barcode is indicative of the protein of interest to which it is fused; (b) immobilizing the library of fusion polypeptides to a surface; (c) contacting the library of fusion polypeptides with a labeled binding reagent; (d) detecting signals indicative of binding between the labeled binding reagent and the protein of interest of at least one fusion polypeptide of the library; (e) cleaving the peptide barcode from the at least one fusion polypeptide comprising the protein of interest; (f) enzymatically conjugating the peptide barcode to a polyglycine-avidin composition described herein, thereby forming a conjugate comprising the peptide barcode and the avidin protein; (g) contacting the conjugate with a biotinylated surface, thereby immobilizing the peptide barcode to the biotinylated surface through the avidin protein; and (h) sequencing the peptide barcode to identify the protein of interest of the at least one fusion polypeptide.

In some aspects, the peptide barcode immobilization strategies described herein can be used in a method for identifying a target analyte in a region of a sample. In some embodiments, the method comprises: (a) contacting a sample with an affinity reagent conjugated to a peptide barcode indicative of a target analyte to which the affinity reagent binds; (b) releasing the peptide barcode from the affinity reagent in a first region of the sample; (c) enzymatically conjugating the peptide barcode to a polyglycine-avidin composition described herein, thereby forming a conjugate comprising the peptide barcode and the avidin protein; (d) contacting the conjugate with a biotinylated surface, thereby immobilizing the peptide barcode to the biotinylated surface through the avidin protein; and (e) sequencing the peptide barcode to identify the target analyte in the first region of the sample.

In some aspects, the peptide barcode immobilization strategies described herein can be used in a method for identifying target analytes in a sample. In some embodiments, the method comprises: (a) contacting a sample with a plurality of affinity reagents conjugated to different peptide barcodes indicative of different target analytes; (b) releasing one or more peptide barcodes from one or more respective affinity reagents in a first region of the sample; (c) releasing one or more peptide barcodes from one or more respective affinity reagents in a second region of the sample; (d) immobilizing the one or more peptide barcodes released from each of the first and second regions of the sample to a surface, where the immobilizing comprises enzymatically conjugating the peptide barcode to a polyglycine-avidin composition described herein, thereby forming a conjugate comprising the peptide barcode and the avidin protein; (e) contacting the conjugate with a biotinylated surface, thereby immobilizing the peptide barcode to the biotinylated surface through the avidin protein; (f) sequencing the one or more peptide barcodes released from each of the first and second regions of the sample.

In some embodiments, a target analyte is a protein. In some embodiments, a target analyte is a monomeric protein. In some embodiments, a target analyte is a multimeric protein. In some embodiments, a target analyte is an antibody. In some embodiments, a target analyte is a receptor. In some embodiments, a target analyte is a ligand. In some embodiments, a target analyte is a cellular component. In some embodiments, a target analyte is a subcellular compartment. In some embodiments, a target analyte is a nucleic acid. In some embodiments, a target analyte is a DNA molecule. In some embodiments, a target analyte is a genomic DNA (gDNA) molecule. In some embodiments, a target analyte is a complementary DNA (cDNA) molecule. In some embodiments, a target analyte is an RNA molecule. In some embodiments, a target analyte is a messenger RNA (mRNA) molecule. In some embodiments, a target analyte is a transfer RNA (tRNA) molecule. In some embodiments, a target analyte is a ribosomal RNA (rRNA) molecule. In some embodiments, a target analyte is a gene transcript.

In some aspects, the peptide barcode immobilization strategies described herein can be used in a method for identifying a therapeutic agent in a region of a sample. In some embodiments, the method comprises: (a) contacting a sample with a therapeutic agent conjugated to a peptide barcode indicative of the therapeutic agent; (b) releasing the peptide barcode from the therapeutic agent in a first region of the sample; (c) enzymatically conjugating the peptide barcode to a polyglycine-avidin composition described herein, thereby forming a conjugate comprising the peptide barcode and the avidin protein; (d) contacting the conjugate with a biotinylated surface, thereby immobilizing the peptide barcode to the biotinylated surface through the avidin protein; and (e) sequencing the peptide barcode to identify the therapeutic agent in the first region of the sample.

In some embodiments, after (b), the peptide barcode remains conjugated to the therapeutic agent in a second region of the sample. In some embodiments, the method further comprises releasing the peptide barcode from the therapeutic agent in the second region of the sample. In some embodiments, the sequencing comprises identifying the therapeutic agent in each of the first and second regions of the sample. In some embodiments, the method further comprises washing the sample between (a) and (b).

In some embodiments, a sample is a biological sample. In some embodiments, a biological sample is derived from a human, a non-human primate, a rodent, an insect, a parasite, or a plant. In some embodiments, a biological sample is fixed. In some embodiments, a biological sample is a cell sample. In some embodiments, a biological sample is a serum sample. In some embodiments, a biological sample is a tissue sample. In some embodiments, a tissue sample is formalin fixed. In some embodiments, a tissue sample is paraffin embedded. In some embodiments, a tissue sample is a formalin-fixed, paraffin-embedded (FFPE) tissue sample. In some embodiments, a tissue sample is a fresh FFPE tissue sample. In some embodiments, a tissue sample is a frozen FFPE tissue sample. In some embodiments, a tissue sample is a fresh-frozen FFPE tissue sample.

In some embodiments, the sequencing comprises identifying the presence of the therapeutic agent in the sample (or a specified region of the sample as described herein). In some embodiments, the sequencing comprises determining a concentration of the therapeutic agent in the sample (or a specified region of the sample as described herein). In some embodiments, the method further comprises evaluating the therapeutic effectiveness of the therapeutic agent. In some embodiments, the therapeutic effectiveness is evaluated based on the presence, concentration, and/or localization of the therapeutic agent in the sample as described herein. In some embodiments, the therapeutic agent comprises an affinity reagent described herein. In some embodiments, the therapeutic agent comprises an antibody-drug conjugate (ADC).

In some aspects, the peptide barcode immobilization strategies described herein can be used in a method of evaluating an antibody-drug conjugate (ADC) in a subject, the method comprising: (a) providing a sample of a subject receiving an ADC, wherein the ADC is conjugated to a peptide barcode; (b) releasing the peptide barcode from the ADC in the sample; (c) enzymatically conjugating the peptide barcode to a polyglycine-avidin composition described herein, thereby forming a conjugate comprising the peptide barcode and the avidin protein; (d) contacting the conjugate with a biotinylated surface, thereby immobilizing the peptide barcode to the biotinylated surface through the avidin protein; (e) sequencing the peptide barcode; and (f) evaluating the ADC based on the sequencing. In some embodiments, the sample is a serum sample of the subject. In some embodiments, the sample is a tissue sample of the subject. In some embodiments, the subject is a human. In some embodiments, the subject is a non-human animal.

In some embodiments, the therapeutic effectiveness is evaluated based on the presence, concentration, and/or localization of the ADC in the sample as described herein. In some embodiments, the therapeutic effectiveness is evaluated based on the duration of time between the subject receiving the ADC and the sample being obtained from the subject. In some embodiments, the therapeutic effectiveness is evaluated by comparing sequencing results with one or more other samples of the subject (e.g., a sample obtained prior to treatment with the ADC, a sample obtained at a different time point following treatment with the ADC). In some embodiments, the method further comprises adjusting (e.g., increasing or decreasing) the dosage amount of the ADC in the therapeutic regimen of the subject based on the sequencing or information derived therefrom. In some embodiments, the evaluating comprises determining a concentration of the ADC in the sample based on the sequencing. In some embodiments, the evaluating comprises comparing the concentration of the ADC in the sample to a control sample. In some embodiments, the control sample is a sample of the subject prior to receiving the ADC. In some embodiments, the control sample is a sample of the subject at a different time point after receiving the ADC.

In some embodiments, (b) comprises: releasing the peptide barcode from the ADC in a first region of the sample, wherein the peptide barcode remains conjugated to the ADC in a second region of the sample. In some embodiments, the method further comprises releasing the peptide barcode from the ADC in the second region of the sample. In some embodiments, the sequencing comprises sequencing the peptide barcode released from the ADC in each of the first and second regions of the sample. In some embodiments, the evaluating comprises: determining a first concentration of the ADC in the first region of the sample based on the sequencing; and determining a second concentration of the ADC in the second region of the sample based on the sequencing.

In some aspects, the peptide barcode immobilization strategies described herein can be used in a method for identifying delivery to a target cell. In some embodiments, the method comprises: (a) contacting a cell with a delivery agent that comprises a polynucleotide, wherein the polynucleotide encodes a protein of interest fused to a peptide barcode; (b) contacting the cell with a cleaving agent configured to cleave the peptide barcode from the protein of interest; (c) enzymatically conjugating the peptide barcode to a polyglycine-avidin composition described herein, thereby forming a conjugate comprising the peptide barcode and the avidin protein; (d) contacting the conjugate with a biotinylated surface, thereby immobilizing the peptide barcode to the biotinylated surface through the avidin protein; and (e) detecting the peptide barcode to identify uptake of the polynucleotide by the cell. In some embodiments, the delivery agent is a lipid nanoparticle that encapsulates the polynucleotide. In some embodiments, the contacting is performed under conditions suitable for expression of the polynucleotide within the cell.

In some aspects, the peptide barcode immobilization strategies described herein can be used in a method for evaluating nanoparticle delivery to a target cell. In some embodiments, the method comprises: (a) contacting a cell with a plurality of nanoparticles having different lipid compositions, wherein each nanoparticle encapsulates a polynucleotide, wherein the polynucleotide encodes a protein of interest fused to a peptide barcode indicative of lipid composition of the nanoparticle; (b) contacting the cell with a cleaving agent configured to cleave the peptide barcode from the protein of interest; (c) enzymatically conjugating the peptide barcode to a polyglycine-avidin composition described herein, thereby forming a conjugate comprising the peptide barcode and the avidin protein; (d) contacting the conjugate with a biotinylated surface, thereby immobilizing the peptide barcode to the biotinylated surface through the avidin protein; and (e) sequencing the peptide barcode to identify the lipid composition of the nanoparticle. In some embodiments, the contacting is performed under conditions suitable for expression of the polynucleotide within the cell.

In some embodiments, a payload fused to a peptide barcode is delivered by a lipid-based delivery system. In some embodiments, the lipid-based delivery system is a lipid nanoparticle (LNP). An LNP carrying a payload fused to a peptide barcode can be used to deliver the payload to a specific cell. In some embodiments, the payload is an mRNA molecule encoding a payload protein and a peptide barcode. Once an LNP delivers an mRNA molecule encoding a payload protein and a peptide barcode to a specific cell, the cell's endogenous translation system translates the mRNA resulting in a protein payload fused to a peptide barcode. The peptide barcode can then be cleaved from the payload protein and sequenced. The presence of sequencing reads associated with a peptide barcode is indicative of the presence of the payload protein within a specific cell. In some embodiments, a peptide barcode further comprises a cell surface receptor. In some embodiments, a cell surface receptor shuttles a peptide barcode to a cell surface for more efficient collection of peptide barcodes. This screening method can be used to screen delivery methods to identify effective and highly specific delivery vehicles.

In some aspects, the peptide barcode immobilization strategies described herein can be used in a method for evaluating protein production. In some embodiments, the method comprises: (a) contacting a cell with an mRNA molecule encoding a protein of interest and a peptide barcode; (b) contacting the cell with a cleaving agent configured to cleave the peptide barcode from the protein of interest; (c) enzymatically conjugating the peptide barcode to a polyglycine-avidin composition described herein, thereby forming a conjugate comprising the peptide barcode and the avidin protein; (d) contacting the conjugate with a biotinylated surface, thereby immobilizing the peptide barcode to the biotinylated surface through the avidin protein; and (e) detecting the peptide barcode to identify production of the protein of interest. In some embodiments, the contacting is performed under conditions suitable for expression of the polynucleotide within the cell.

To screen for mRNA protein production, an mRNA can be designed to encode a protein of interest and a peptide barcode. The mRNA can then be delivered to a specific cell and the cell's endogenous translation system can translate the mRNA to produce the protein of interest fused to the peptide barcode. The peptide barcode can be cleaved from the protein of interest using any appropriate cleaving agent (e.g., described herein or known in the art). The peptide barcode can then be collected and sequenced. The presence of sequencing reads associated with a specific barcode is indicative of translation of the protein of interest to which the peptide barcode was fused. This method can be multiplexed to screen mRNA translation efficiency of thousands of mRNA molecules at once. This method can be further modified to screen delivery of mRNA vaccine candidates. An mRNA vaccine candidate encoding a vaccine protein or peptide can be modified to encode a peptide barcode in addition to a vaccine candidate. Presence of sequencing reads associated with a peptide barcode is indicative of production of a vaccine candidate in a specific cell.

In some aspects, the present disclosure relates to a method of peptide sequencing, comprising: obtaining any fusion polypeptide described herein; enriching the fusion polypeptide by affinity capture; contacting the fusion polypeptide with a sortase; contacting the fusion polypeptide with an endoproteinase to isolate the barcode; and sequencing the barcode. In some embodiments, the sequence of the barcode is indicative of the identity of the protein of interest.

In some aspects, the present disclosure relates to a method of tracking a protein of interest in a subject, the method comprising: administering to the subject a messenger RNA (mRNA) molecule encoding any fusion protein described herein; extracting from the subject a tissue of interest; and executing any method described herein. In some embodiments, the administering comprises administering a viral particles of lipid nanoparticle comprising the mRNA. In some embodiments, the tissue of interest is liver tissue, kidney tissue, brain tissue, lung tissue, cardiac tissue, or blood.

In some aspects, the present disclosure relates to a method of peptide sequencing, the method comprising: obtaining any library described herein; contacting the first subset of fusion polypeptides with a molecule that binds to the first affinity tag; contacting the first subset of fusion polypeptides with a sortase; contacting the first subset of fusion polypeptides with an endoproteinase to isolate the barcode of the first subset of fusion polypeptides; sequencing the barcode of the first subset of fusion polypeptides; contacting the second subset of fusion polypeptides with a molecule that binds to the second affinity tag; contacting the second subset of fusion polypeptides with a sortase; contacting the second subset of fusion polypeptides with an endoproteinase to isolate the barcode of the second subset of fusion polypeptides; and sequencing the barcode of the second subset of fusion polypeptides.

In some embodiments, the method further comprises contacting the third subset of fusion polypeptides with a molecule that binds to the third affinity tag; contacting the third subset of fusion polypeptides with a sortase; contacting the third subset of fusion polypeptides with an endoproteinase to isolate the barcode of the third subset of fusion polypeptides; and sequencing the barcode of the third subset of fusion polypeptides.

In some embodiments, the method further comprises contacting the fourth subset of fusion polypeptides with a molecule that binds to the fourth affinity tag; contacting the fourth subset of fusion polypeptides with a sortase; contacting the fourth subset of fusion polypeptides with an endoproteinase to isolate the barcode of the fourth subset of fusion polypeptides; and sequencing the barcode of the fourth subset of fusion polypeptides.

In some embodiments, the barcodes within the first subset of fusion polypeptides, the second subset of fusion polypeptides, the third subset of fusion polypeptides, and the fourth subset of fusion polypeptides are each different within subsets of fusion polypeptides but the same across subsets of fusion polypeptides.

A person having ordinary skill in the art will understand that many different affinity tags known in the art beyond those described herein can be used in the methods described herein. A person having ordinary skill in the art will also understand that any number of subsets of fusion polypeptides having affinity tags, and not only the specific subsets described herein, can be used in the methods described herein. A person having ordinary skill in the art will also understand that a set of any number of unique barcode sequences, and not only the specific barcode sequences described herein, can be used in the methods described herein, e.g., the plexity expansion methods described herein.

Functionalization with Luminescent Label

In some aspects, the disclosure provides compositions that can be enzymatically conjugated to a fusion polypeptide comprising a protein of interest for labeling. In some embodiments, the composition comprises: an avidin protein comprising at least two biotin binding sites; a first biotin moiety bound to at least one biotin binding site on the avidin protein; and a polyglycine peptide attached to the first biotin moiety through a linker, where the linker comprises a polyethylene glycol (PEG) moiety. In some embodiments, the composition further comprises: a second biotin moiety bound to at least one biotin binding site on the avidin protein; and a nucleic acid attached to the second biotin moiety, where the nucleic acid comprises one or more luminescent labels. In this way, conjugation of the composition to the fusion polypeptide provides a luminescently labeled protein of interest.

In some embodiments, the nucleic acid comprises at least two fluorophore dyes. In some embodiments, the nucleic acid comprises between about 1 and about 20 fluorophore dyes. In some embodiments, the nucleic acid comprises 2-10, 2-5, 3-10, or 5-15 fluorophore dyes. In some embodiments, the nucleic acid comprises 1, 2, 3, 4, 5, 6, 7, 8, 9, or 10 fluorophore dyes. In some embodiments, each fluorophore dye is attached to a different attachment site on the nucleic acid. In some embodiments, the nucleic acid comprises at least one luminescent label as described herein.

In some embodiments, the nucleic acid comprises DNA, RNA, or a combination thereof. In some embodiments, the nucleic acid is a double-stranded nucleic acid. In some embodiments, the nucleic acid is a single-stranded nucleic acid. In some embodiments, the nucleic acid is between about 5 and about 100 bases or base pairs in length. In some embodiments, the nucleic acid is between about 5 and about 50 bases or base pairs in length. In some embodiments, the nucleic acid is between about 10 and about 75 bases or base pairs in length. In some embodiments, the nucleic acid is between about 20 and about 60 bases or base pairs in length.

In some embodiments, the first biotin moiety is covalently attached to the PEG moiety. In some embodiments, the PEG moiety forms a covalent linkage group between the polyglycine peptide and the first biotin moiety. In some embodiments, the second biotin moiety comprises a bis-biotin moiety bound to two biotin binding sites on the avidin protein. In some embodiments, each of the first and second biotin moiety comprises a bis-biotin moiety bound to two biotin binding sites on the avidin protein. Accordingly, in some embodiments, the avidin protein comprises four biotin binding sites, the first biotin moiety comprises a bis-biotin moiety bound to two biotin binding sites on the avidin protein, and the second biotin moiety comprises a bis-biotin moiety bound to two biotin binding sites on the avidin protein. See, e.g., an example polyglycine-streptavidin composition used for conjugation in the workflow depicted in FIG. 1B.

In some aspects, the disclosure provides methods comprising: (a) contacting a fusion polypeptide comprising a protein of interest with a composition comprising a polyglycine peptide and one or more luminescent labels as described herein; and (b) enzymatically conjugating the protein of interest to the polyglycine peptide, thereby forming a conjugate comprising the protein of interest and the one or more luminescent labels.

In some embodiments, the method further comprises: (c) contacting the conjugate with a surface-immobilized compound; and (d) monitoring a luminescent signal for signal pulses corresponding to binding interactions between the protein of interest and the surface-immobilized compound. In some embodiments, the surface-immobilized compound is a surface-immobilized polypeptide. In some embodiments, the surface-immobilized compound is a surface-immobilized small molecule (e.g., a compound of less than 1,000 Da). In some embodiments, the protein of interest is an affinity reagent. In some embodiments, the protein of interest is an antibody. In some embodiments, the protein of interest is an adaptor protein (e.g., a terminal amino acid binding protein).

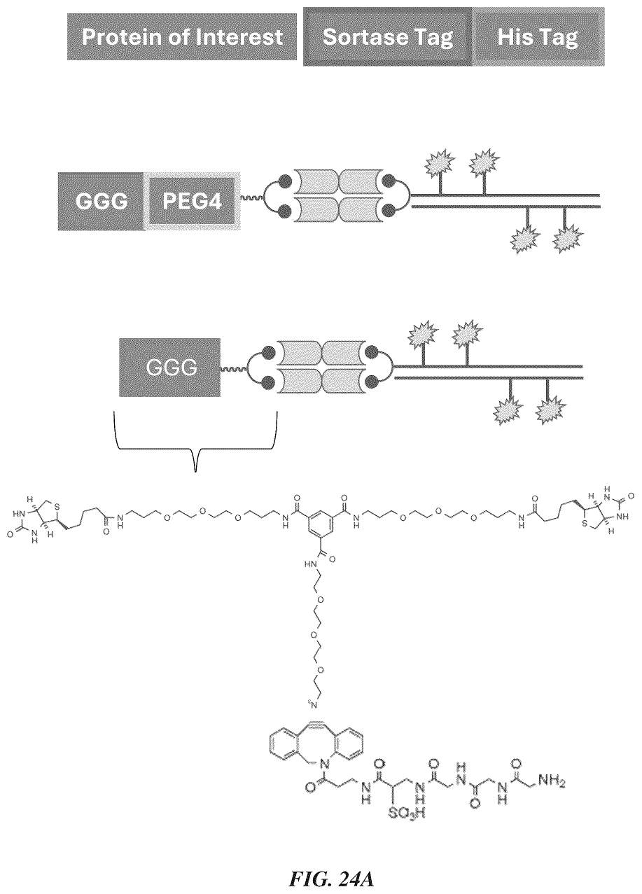

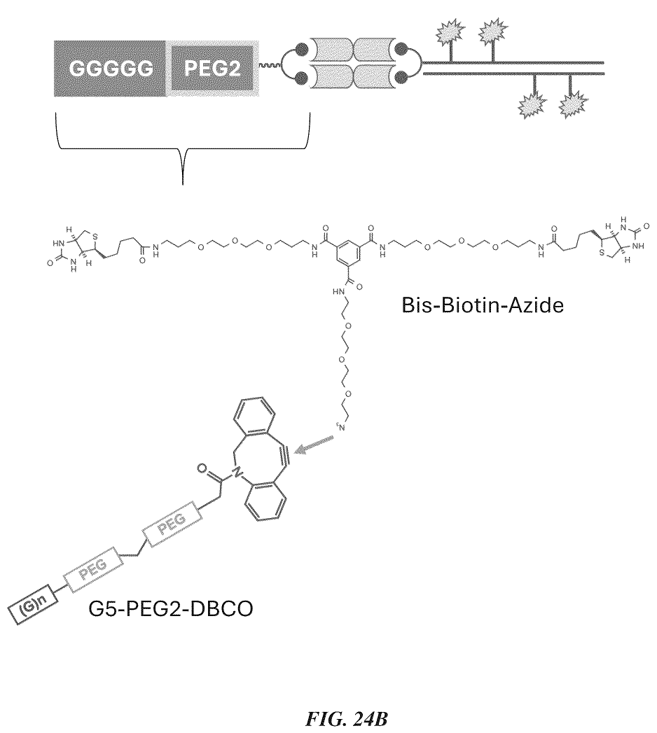

FIGS. 24A-24B depict example polyglycine-streptavidin compositions comprising luminescent labels for labeling a protein of interest as described herein.

### Methods of Using Luminescently Labeled Molecules

In some aspects, the labeling strategies described herein can be used in a method for screening for modulators of a protein of interest. In some embodiments, the method comprises: (a) contacting a fusion polypeptide comprising a protein of interest with a composition comprising a polyglycine peptide and one or more luminescent labels as described herein; (b) enzymatically conjugating the protein of interest to the polyglycine peptide, thereby forming a conjugate comprising the protein of interest and the one or more luminescent labels; (c) contacting the conjugate with a substrate comprising a library of different compounds immobilized to a surface of the substrate; and (d) monitoring a luminescent signal for signal pulses corresponding to binding interactions between the protein of interest and at least one compound of the library.

In some embodiments, the library of different compounds comprises at least two, at least five, at least 10, at least 20, at least 30, at least 50, at least 75, at least 100, at least 200, at least 250, at least 500, or at least 1,000 different compounds. In some embodiments, the library of different compounds comprises between about 10 and about 500 different compounds. In some embodiments, the library of different compounds comprises between about 100 and about 1,000 different compounds. In some embodiments, the library of different compounds comprises between about 1,000 and about 10,000 different compounds.

In some embodiments, the library of different compounds comprises a plurality of different polypeptides. In some embodiments, the library of different compounds comprises a plurality of different small molecule compounds. In some embodiments, each of the different small molecule compounds of the library is less than 1,000 Da (e.g., 50-500, 10-1,000, 500-1,000, or 100-500 Da).

In some aspects, the labeling strategies described herein can be used in a method for polypeptide sequencing. In some embodiments, the method comprises: (a) contacting a fusion polypeptide comprising a protein of interest (e.g., an affinity reagent) with a composition comprising a polyglycine peptide and one or more luminescent labels as described herein; (b) enzymatically conjugating the protein of interest to the polyglycine peptide, thereby forming a conjugate comprising the protein of interest and the one or more luminescent labels; (c) contacting the conjugate with a polypeptide; and (d) monitoring a luminescent signal for signal pulses corresponding to binding interactions between the protein of interest and successive amino acids exposed at a terminus of the polypeptide while the polypeptide is being degraded.

In some embodiments, the contacting of (c) comprises contacting the polypeptide with the conjugate and one or more luminescently labeled amino acid recognition molecules. In some embodiments, the method is performed in a reaction mixture comprising one or more cleaving reagents that degrade the polypeptide. In some embodiments, the one or more cleaving reagents comprise one or more aminopeptidases.

### Labels

In some embodiments, the disclosure provides methods of conjugating a protein of interest to a luminescent label. As used herein, a luminescent label is a molecule that absorbs one or more photons and may subsequently emit one or more photons after one or more time durations. In some embodiments, the term is used interchangeably with “label,” “detectable label,” or “luminescent molecule” depending on context. A luminescent label in accordance with certain embodiments described herein may refer to a luminescent label of a protein of interest (e.g., an affinity reagent), a luminescent label of an amino acid recognizer, a luminescent label of a cleaving reagent (e.g., a peptidase, such as an aminopeptidase), or a luminescent label of another labeled composition described herein.

In some embodiments, a luminescent label comprises a first chromophore and a second chromophore. In some embodiments, an excited state of the first chromophore is capable of relaxation via an energy transfer to the second chromophore. In some embodiments, the energy transfer is a Förster resonance energy transfer (FRET). Such a FRET pair may be useful for providing a luminescent label with properties that make the label easier to differentiate from amongst a plurality of luminescent labels in a mixture, or for providing a binding-induced fluorescence that limits background fluorescence as described elsewhere herein. In yet other embodiments, a FRET pair comprises a first chromophore of a first luminescent label and a second chromophore of a second luminescent label. In certain embodiments, the FRET pair may absorb excitation energy in a first spectral range and emit luminescence in a second spectral range.

In some embodiments, a luminescent label refers to a fluorophore or a dye. Typically, a luminescent label comprises an aromatic or heteroaromatic compound and can be a pyrene, anthracene, naphthalene, naphthylamine, acridine, stilbene, indole, benzindole, oxazole, carbazole, thiazole, benzothiazole, benzoxazole, phenanthridine, phenoxazine, porphyrin, quinoline, ethidium, benzamide, cyanine, carbocyanine, salicylate, anthranilate, coumarin, fluorescein, rhodamine, xanthene, or other like compounds.

In some embodiments, a luminescent label comprises a dye selected from one or more of the following: 5/6-Carboxyrhodamine 6G, 5-Carboxyrhodamine 6G, 6-Carboxyrhodamine 6G, 6-TAMRA, Abberior® STAR 440SXP, Abberior® STAR 470SXP, Abberior® STAR 488, Abberior® STAR 512, Abberior® STAR 520SXP, Abberior® STAR 580, Abberior® STAR 600, Abberior® STAR 635, Abberior® STAR 635P, Abberior® STAR RED, Alexa Fluor® 350, Alexa Fluor® 405, Alexa Fluor® 430, Alexa Fluor® 480, Alexa Fluor® 488, Alexa Fluor® 514, Alexa Fluor® 532, Alexa Fluor® 546, Alexa Fluor® 555, Alexa Fluor® 568, Alexa Fluor® 594, Alexa Fluor® 610-X, Alexa Fluor® 633, Alexa Fluor® 647, Alexa Fluor® 660, Alexa Fluor® 680, Alexa Fluor® 700, Alexa Fluor® 750, Alexa Fluor® 790, AMCA, ATTO 390, ATTO 425, ATTO 465, ATTO 488, ATTO 495, ATTO 514, ATTO 520, ATTO 532, ATTO 542, ATTO 550, ATTO 565, ATTO 590, ATTO 610, ATTO 620, ATTO 633, ATTO 647, ATTO 647N, ATTO 655, ATTO 665, ATTO 680, ATTO 700, ATTO 725, ATTO 740, ATTO Oxa12, ATTO Rho101, ATTO Rho11, ATTO Rho12, ATTO Rho13, ATTO Rho14, ATTO Rho3B, ATTO Rho6G, ATTO Thio12, BD Horizon™ V450, BODIPY® 493/501, BODIPY® 530/550, BODIPY® 558/568, BODIPY® 564/570, BODIPY® 576/589, BODIPY® 581/591, BODIPY® 630/650, BODIPY® 650/665, BODIPY® FL, BODIPY® FL-X, BODIPY® R6G, BODIPY® TMR, BODIPY® TR, CAL Fluor® Gold 540, CAL Fluor® Green 510, CAL Fluor® Orange 560, CAL Fluor® Red 590, CAL Fluor® Red 610, CAL Fluor® Red 615, CAL Fluor® Red 635, Cascade® Blue, CFTM350, CFTM405M, CFTM405S, CFTM488A, CFTM514, CFTM532, CFTM543, CFTM546, CFTM555, CFTM568, CFTM594, CFTM620R, CFTM633, CFTM633-V1, CFTM640R, CFTM640R-V1, CFTM640R-V2, CFTM660C, CFTM660R, CFTM680, CFTM680R, CFTM680R-V1, CFTM750, CFTM770, CFTM790, Chromeo™ 642, Chromis 425N, Chromis 500N, Chromis 515N, Chromis 530N, Chromis 550A, Chromis 550C, Chromis 550Z, Chromis 560N, Chromis 570N, Chromis 577N, Chromis 600N, Chromis 630N, Chromis 645A, Chromis 645C, Chromis 645Z, Chromis 678A, Chromis 678C, Chromis 678Z, Chromis 770A, Chromis 770C, Chromis 800A, Chromis 800C, Chromis 830A, Chromis 830C, Cy®3, Cy®3.5, Cy®3B, Cy®5, Cy®5.5, Cy®7, DyLight® 350, DyLight® 405, DyLight® 415-Col, DyLight® 425Q, DyLight® 485-LS, DyLight® 488, DyLight® 504Q, DyLight® 510-LS, DyLight® 515-LS, DyLight® 521-LS, DyLight® 530-R2, DyLight® 543Q, DyLight® 550, DyLight® 554-R0, DyLight® 554-R1, DyLight® 590-R2, DyLight® 594, DyLight® 610-B1, DyLight® 615-B2, DyLight® 633, DyLight® 633-B1, DyLight® 633-B2, DyLight® 650, DyLight® 655-B1, DyLight® 655-B2, DyLight® 655-B3, DyLight® 655-B4, DyLight® 662Q, DyLight® 675-B1, DyLight® 675-B2, DyLight® 675-B3, DyLight® 675-B4, DyLight® 679-C5, DyLight® 680, DyLight® 683Q, DyLight® 690-B1, DyLight® 690-B2, DyLight® 696Q, DyLight® 700-B1, DyLight® 700-B1, DyLight® 730-B1, DyLight® 730-B2, DyLight® 730-B3, DyLight®730-B4, DyLight® 747, DyLight® 747-B1, DyLight® 747-B2, DyLight® 747-B3, DyLight® 747-B4, DyLight® 755, DyLight® 766Q, DyLight® 775-B2, DyLight® 775-B3, DyLight®775-B4, DyLight® 780-B1, DyLight® 780-B2, DyLight® 780-B3, DyLight® 800, DyLight®830-2, Dyomics-350, Dyomics-350XL, Dyomics-360XL, Dyomics-370XL, Dyomics-375XL, Dyomics-380XL, Dyomics-390XL, Dyomics-405, Dyomics-415, Dyomics-430, Dyomics-431, Dyomics-478, Dyomics-480XL, Dyomics-481XL, Dyomics-485XL, Dyomics-490, Dyomics-495, Dyomics-505, Dyomics-510XL, Dyomics-511XL, Dyomics-520XL, Dyomics-521XL, Dyomics-530, Dyomics-547, Dyomics-547P1, Dyomics-548, Dyomics-549, Dyomics-549P1, Dyomics-550, Dyomics-554, Dyomics-555, Dyomics-556, Dyomics-560, Dyomics-590, Dyomics-591, Dyomics-594, Dyomics-601XL, Dyomics-605, Dyomics-610, Dyomics-615, Dyomics-630, Dyomics-631, Dyomics-632, Dyomics-633, Dyomics-634, Dyomics-635, Dyomics-636, Dyomics-647, Dyomics-647P1, Dyomics-648, Dyomics-648P1, Dyomics-649, Dyomics-649P1, Dyomics-650, Dyomics-651, Dyomics-652, Dyomics-654, Dyomics-675, Dyomics-676, Dyomics-677, Dyomics-678, Dyomics-679P1, Dyomics-680, Dyomics-681, Dyomics-682, Dyomics-700, Dyomics-701, Dyomics-703, Dyomics-704, Dyomics-730, Dyomics-731, Dyomics-732, Dyomics-734, Dyomics-749, Dyomics-749P1, Dyomics-750, Dyomics-751, Dyomics-752, Dyomics-754, Dyomics-776, Dyomics-777, Dyomics-778, Dyomics-780, Dyomics-781, Dyomics-782, Dyomics-800, Dyomics-831, eFluor® 450, Eosin, FITC, Fluorescein, HiLyte™ Fluor 405, HiLyte™ Fluor 488, HiLyte™ Fluor 532, HiLyte™ Fluor 555, HiLyte™ Fluor 594, HiLyte™ Fluor 647, HiLyte™ Fluor 680, HiLyte™ Fluor 750, IRDye® 680LT, IRDye® 750, IRDye® 800CW, JOE, LightCycler® 640R, LightCycler® Red 610, LightCycler® Red 640, LightCycler® Red 670, LightCycler® Red 705, Lissamine Rhodamine B, Napthofluorescein, Oregon Green® 488, Oregon Green® 514, Pacific Blue™, Pacific Green™, Pacific Orange™, PET, PF350, PF405, PF415, PF488, PF505, PF532, PF546, PF555P, PF568, PF594, PF610, PF633P, PF647P, Quasar® 570, Quasar® 670, Quasar® 705, Rhodamine 123, Rhodamine 6G, Rhodamine B, Rhodamine Green, Rhodamine Green-X, Rhodamine Red, ROX, Seta™ 375, Seta™ 470, Seta™ 555, Seta™ 632, Seta™ 633, Seta™ 650, Seta™ 660, Seta™ 670, Seta™ 680, Seta™ 700, Seta™ 750, Seta™ 780, Seta™ APC-780, Seta™ PerCP-680, Seta™ R-PE-670, Seta™ 646, SeTau 380, SeTau 425, SeTau 647, SeTau 405, Square 635, Square 650, Square 660, Square 672, Square 680, Sulforhodamine 101, TAMRA, TET, Texas Red®, TMR, TRITC, Yakima Yellow™, Zenon®, Zy3, Zy5, Zy5.5, and Zy7.

In some aspects, the disclosure provides methods and compositions for polypeptide analysis (e.g., amino acid recognition) based on one or more luminescence properties of a luminescent label. In some embodiments, a luminescent label is identified based on luminescence lifetime, luminescence intensity, brightness, absorption spectra, emission spectra, luminescence quantum yield, or a combination of two or more thereof. In some embodiments, a plurality of types of luminescent labels can be distinguished from each other based on a difference in luminescence lifetime, luminescence intensity, brightness, absorption spectra, emission spectra, luminescence quantum yield, or combinations of two or more thereof.

In some embodiments, luminescence is detected by exposing a luminescent label to a series of separate light pulses and evaluating the timing or other properties of each photon that is emitted from the label. In some embodiments, information for a plurality of photons emitted sequentially from a label is aggregated and evaluated to identify the label and thereby identify an associated barcode site. In some embodiments, a luminescence lifetime of a label is determined from a plurality of photons that are emitted sequentially from the label, and the luminescence lifetime can be used to identify the label. In some embodiments, a luminescence intensity of a label is determined from a plurality of photons that are emitted sequentially from the label, and the luminescence intensity can be used to identify the label. In some embodiments, a luminescence lifetime and luminescence intensity of a label is determined from a plurality of photons that are emitted sequentially from the label, and the luminescence lifetime and luminescence intensity can be used to identify the label.

In some aspects of the disclosure, a single molecule is exposed to a plurality of separate light pulses and a series of emitted photons are detected and analyzed. In some embodiments, the series of emitted photons provides information about the single molecule that is present and that does not change in the mixture over the course of an experiment. However, in some embodiments, the series of emitted photons provides information about a series of different molecules that are present at different times in the mixture (e.g., as a reaction or process progresses).

In certain embodiments, a luminescent label absorbs one photon and emits one photon after a time duration. In some embodiments, the luminescence lifetime of a label can be determined or estimated by measuring the time duration. In some embodiments, the luminescence lifetime of a label can be determined or estimated by measuring a plurality of time durations for multiple pulse events and emission events. In some embodiments, the luminescence lifetime of a label can be differentiated amongst the luminescence lifetimes of a plurality of types of labels by measuring the time duration. In some embodiments, the luminescence lifetime of a label can be differentiated amongst the luminescence lifetimes of a plurality of types of labels by measuring a plurality of time durations for multiple pulse events and emission events. In certain embodiments, a label is identified or differentiated amongst a plurality of types of labels by determining or estimating the luminescence lifetime of the label. In certain embodiments, a label is identified or differentiated amongst a plurality of types of labels by differentiating the luminescence lifetime of the label amongst a plurality of the luminescence lifetimes of a plurality of types of labels.

Determination of a luminescence lifetime of a luminescent label can be performed using any suitable method (e.g., by measuring the lifetime using a suitable technique or by determining time-dependent characteristics of emission). In some embodiments, determining the luminescence lifetime of one label comprises determining the lifetime relative to another label. In some embodiments, determining the luminescence lifetime of a label comprises determining the lifetime relative to a reference. In some embodiments, determining the luminescence lifetime of a label comprises measuring the lifetime (e.g., fluorescence lifetime). In some embodiments, determining the luminescence lifetime of a label comprises determining one or more temporal characteristics that are indicative of lifetime. In some embodiments, the luminescence lifetime of a label can be determined based on a distribution of a plurality of emission events (e.g., 1, 2, 3, 4, 5, 6, 7, 8, 9, 10, 11, 12, 13, 14, 15, 16, 17, 18, 19, 20, 30, 40, 50, 60, 70, 80, 90, 100, or more emission events) occurring across one or more time-gated windows relative to an excitation pulse. For example, a luminescence lifetime of a label can be distinguished from a plurality of labels having different luminescence lifetimes based on the distribution of photon arrival times measured with respect to an excitation pulse.

It should be appreciated that a luminescence lifetime of a luminescent label is indicative of the timing of photons emitted after the label reaches an excited state and the label can be distinguished by information indicative of the timing of the photons. Some embodiments may include distinguishing a label from a plurality of labels based on the luminescence lifetime of the label by measuring times associated with photons emitted by the label. The distribution of times may provide an indication of the luminescence lifetime which may be determined from the distribution. In some embodiments, the label is distinguishable from the plurality of labels based on the distribution of times, such as by comparing the distribution of times to a reference distribution corresponding to a known label. In some embodiments, a value for the luminescence lifetime is determined from the distribution of times.

As used herein, in some embodiments, luminescence intensity refers to the number of emitted photons per unit time that are emitted by a luminescent label which is being excited by delivery of a pulsed excitation energy. In some embodiments, the luminescence intensity refers to the detected number of emitted photons per unit time that are emitted by a label which is being excited by delivery of a pulsed excitation energy and are detected by a particular sensor or set of sensors.

As used herein, in some embodiments, brightness refers to a parameter that reports on the average emission intensity per luminescent label. Thus, in some embodiments, “emission intensity” may be used to generally refer to brightness of a composition comprising one or more labels. In some embodiments, brightness of a label is equal to the product of its quantum yield and extinction coefficient.

As used herein, in some embodiments, luminescence quantum yield refers to the fraction of excitation events at a given wavelength or within a given spectral range that lead to an emission event and is typically less than 1. In some embodiments, the luminescence quantum yield of a luminescent label described herein is between 0 and about 0.001, between about 0.001 and about 0.01, between about 0.01 and about 0.1, between about 0.1 and about 0.5, between about 0.5 and 0.9, or between about 0.9 and 1. In some embodiments, a label is identified by determining or estimating the luminescence quantum yield.

As used herein, in some embodiments, an excitation energy is a pulse of light from a light source. In some embodiments, an excitation energy is in the visible spectrum. In some embodiments, an excitation energy is in the ultraviolet spectrum. In some embodiments, an excitation energy is in the infrared spectrum. In some embodiments, an excitation energy is at or near the absorption maximum of a luminescent label from which a plurality of emitted photons are to be detected. In certain embodiments, the excitation energy is between about 500 nm and about 700 nm (e.g., between about 500 nm and about 600 nm, between about 600 nm and about 700 nm, between about 500 nm and about 550 nm, between about 550 nm and about 600 nm, between about 600 nm and about 650 nm, or between about 650 nm and about 700 nm). In certain embodiments, an excitation energy may be monochromatic or confined to a spectral range. In some embodiments, a spectral range has a range of between about 0.1 nm and about 1 nm, between about 1 nm and about 2 nm, or between about 2 nm and about 5 nm. In some embodiments, a spectral range has a range of between about 5 nm and about 10 nm, between about 10 nm and about 50 nm, or between about 50 nm and about 100 nm.

### Polypeptide Sequencing

In some aspects, the disclosure provides methods of polypeptide analysis (e.g., polypeptide sequencing). In some embodiments, a method of polypeptide analysis comprises contacting a polypeptide with at least one amino acid recognizer; monitoring a signal for signal pulses corresponding to interactions between the polypeptide and the at least one amino acid recognizer; and determining at least one chemical characteristic of the polypeptide based on a characteristic pattern in the signal.

Compositions and methods for performing polypeptide sequencing and analyzing data obtained therefrom are described more fully in PCT International Publication No.
WO2020/102741A1, filed Nov. 15, 2019, PCT International Publication No.
WO2021/236983A2, filed May 20, 2021, PCT International Publication No.
WO2023/122769A2, filed Dec. 22, 2022, PCT International Publication No.
WO2024/031031A2, filed Aug. 3, 2023, PCT International Publication No.
WO2024/086832A1, filed Oct. 20, 2023, PCT International Publication No.
WO2025/101639A3, filed Nov. 6, 2024, PCT International Publication No.
WO2025/147658A1, filed Jan. 3, 2025, and PCT International Application No.: PCT/US2025/042267, filed Aug. 15, 2025, each of which is incorporated by reference in its entirety.

In some embodiments, polypeptide sequencing in accordance with the disclosure can be carried out by dynamic sequencing methods in which polypeptides are exposed to a reaction mixture comprising one or more amino acid recognizers and one or more cleaving reagents (see, e.g., PCT International Publication Nos. WO 2020/102741 A1, WO 2021/236983 A2, WO 2023/122769 A2, WO 2024/031031 A2, WO 2024/086832 A1, WO 2025/101639 A3, and WO 2025/147658 A1, the relevant contents of which are incorporated herein by reference in their entirety). In some embodiments, polypeptide sequencing in accordance with the disclosure can be carried out by controlled cleavage methods in which polypeptides are subjected to iterative cycles of amino acid recognition and amino acid cleavage (see, e.g., PCT International Application No.: PCT/US2025/042267, filed Aug. 15, 2025, the relevant contents of which are incorporated herein by reference in their entirety).

A non-limiting example of polypeptide structure analysis by detecting single molecule binding interactions during a polypeptide degradation process is illustrated in FIG. 1C. An example signal trace is shown depicting different association (e.g., binding) events at times corresponding to changes in the signal. As shown, an association event between an amino acid recognizer and a terminal end of a polypeptide produces a change in magnitude of the signal that persists for a duration of time. Different association events are illustrated for different amino acids exposed at the terminal end of the polypeptide. As described herein, an amino acid that is “exposed” at the terminus of a polypeptide is an amino acid that is still attached to the polypeptide and that becomes the terminal amino acid upon removal of the prior terminal amino acid during degradation (e.g., either alone or along with one or more additional amino acids).

As generically depicted, the association events between amino acid recognizers and different types of amino acids at the terminal end of the polypeptide produce distinctive changes in the signal, referred to herein as a characteristic pattern, which may be used to determine chemical characteristics of the polypeptide. In some embodiments, a characteristic pattern corresponding to one type of terminal amino acid can be used to determine structural information for the terminal amino acid and one or more amino acids contiguous to the terminal amino acid. Accordingly, in some embodiments, a characteristic pattern corresponding to one type of terminal amino acid can be used to determine structural information for at least two (e.g., at least three, at least four, at least five, two, three, four, or between two and five) amino acids of a polypeptide.

In some embodiments, a transition from one characteristic pattern to another is indicative of amino acid cleavage. As used herein, in some embodiments, amino acid cleavage refers to the removal of at least one amino acid from a terminus of a polypeptide (e.g., the removal of at least one terminal amino acid from the polypeptide). In some embodiments, amino acid cleavage is determined by inference based on a time duration between characteristic patterns. In some embodiments, amino acid cleavage is determined by detecting a change in signal produced by association of a labeled cleaving reagent with an amino acid at the terminus of the polypeptide. As amino acids are sequentially cleaved from the terminus of the polypeptide during degradation, a series of changes in magnitude, or a series of signal pulses, is detected.

In some embodiments, signal data can be analyzed to extract signal pulse information by applying threshold levels to one or more parameters of the signal data. For example, in some embodiments, a threshold magnitude level may be applied to the signal data of a signal trace. In some embodiments, the threshold magnitude level is a minimum difference between a signal detected at a point in time and a baseline determined for a given set of data. In some embodiments, a signal pulse is assigned to each portion of the data that is indicative of a change in magnitude exceeding the threshold magnitude level and persisting for a duration of time. In some embodiments, a threshold time duration may be applied to a portion of the data that satisfies the threshold magnitude level to determine whether a signal pulse is assigned to that portion. For example, experimental artifacts may give rise to a change in magnitude exceeding the threshold magnitude level but that does not persist for a duration of time sufficient to assign a signal pulse with a desired confidence (e.g., transient association events which could be non-discriminatory for amino acid type, non-specific detection events such as diffusion into an observation region or reagent sticking within an observation region). Accordingly, in some embodiments, a signal pulse is extracted from signal data based on a threshold magnitude level and a threshold time duration.

In some embodiments, a peak in magnitude of a signal pulse is determined by averaging the magnitude detected over a duration of time that persists above the threshold magnitude level. It should be appreciated that, in some embodiments, a “signal pulse” as used herein can refer to a change in signal data that persists for a duration of time above a baseline (e.g., raw signal data), or to signal pulse information extracted therefrom (e.g., processed signal data).

In some embodiments, signal pulse information can be analyzed to identify different types of amino acids in a polypeptide based on different characteristic patterns in a series of signal pulses. For example, as shown in FIG. 1C, the signal pulse information is indicative of different types of amino acids at a terminal end of a polypeptide (e.g., arginine, leucine, isoleucine, phenylalanine). By way of example, the signal pulses detected at the earliest time points provide information indicative of (at least) arginine at the terminus of the polypeptide based on a first characteristic pattern, and the signal pulses detected at the latest time points provide information indicative of at least phenylalanine at the terminus of the polypeptide based on a second characteristic pattern.

In some embodiments, each signal pulse of a characteristic pattern comprises a pulse duration corresponding to an association event between an amino acid recognizer and an amino acid ligand. In some embodiments, the pulse duration is characteristic of a dissociation rate of binding. In some embodiments, each signal pulse of a characteristic pattern is separated from another signal pulse of the characteristic pattern by an interpulse duration. In some embodiments, the interpulse duration is characteristic of an association rate of binding. In some embodiments, a change in magnitude in a signal can be determined for a signal pulse based on a difference between baseline and the peak of a signal pulse. In some embodiments, a characteristic pattern is determined based on pulse duration. In some embodiments, a characteristic pattern is determined based on pulse duration and interpulse duration. In some embodiments, a characteristic pattern is determined based on any one or more of pulse duration, interpulse duration, and change in magnitude.

Accordingly, as illustrated by FIG. 1C, in some embodiments, polypeptide analysis is performed by detecting a series of signal pulses indicative of association of one or more amino acid recognizers with successive amino acids exposed at the terminus of a polypeptide in an ongoing degradation reaction. The series of signal pulses can be analyzed to determine characteristic patterns in the series of signal pulses, and the time course of characteristic patterns can be used to determine chemical characteristics throughout an amino acid sequence of the polypeptide.

In some embodiments, a plurality of single-molecule sequencing reactions are performed in parallel in an array of sample wells. In some embodiments, an array comprises between about 10,000 and about 1,000,000 sample wells. The volume of a sample well may be between about 10-21 liters and about 10-15 liters, in some implementations. Because the sample well has a small volume, detection of single-molecule events may be possible as only about one polypeptide may be within a sample well at any given time. Statistically, some sample wells may not contain a single-molecule sequencing reaction and some may contain more than one single polypeptide molecule. However, an appreciable number of sample wells may each contain a single-molecule reaction (e.g., at least 30% in some embodiments), so that single-molecule analysis can be carried out in parallel for a large number of sample wells.

### Kits

Aspects of the present disclosure relate to a kit, comprising any composition described herein, any fusion polypeptide described herein, or any library described herein, and materials and/or reagents for executing any method described herein.

In some embodiments, the kit further comprises instructions for executing any method described herein.

### EXAMPLES

### Example 1. Functionalization of Peptide Barcode for Surface Immobilization

In recent years, protein/peptide barcoding has gained attention as a powerful method for advancing protein analysis. This approach leverages the unique ability of short peptide sequences to encode information, providing an efficient and flexible means of tracking and characterizing proteins. Unlike traditional labeling techniques, peptide barcodes can be easily genetically encoded, offering a straightforward way to label proteins within complex biological systems without disrupting their native function. This versatility has made protein barcoding an increasingly valuable tool in proteomics and functional genomics, enabling more precise studies of protein behavior and interactions in a variety of experimental contexts.

Protein barcodes have already been developed and applied in a variety of settings, leveraging the use of mass spectrometry for detection and decoding. For instance, “flycodes” have been used in nanobody screening to rapidly assess protein interactions, and abiotic peptides have been employed for large-scale screening of small molecule libraries. Despite these advances, several challenges remain, particularly in the ability to directly read protein barcode sequences with quantitative accuracy and single-molecule resolution. Ionization efficiency can vary between different peptide sequences, and signal overlap can complicate interpretation11. Furthermore, mass spectrometry requires expensive equipment and extensive expertise to generate and analyze data. This gap has hindered the broader application of peptide barcoding in proteomics and functional screening.

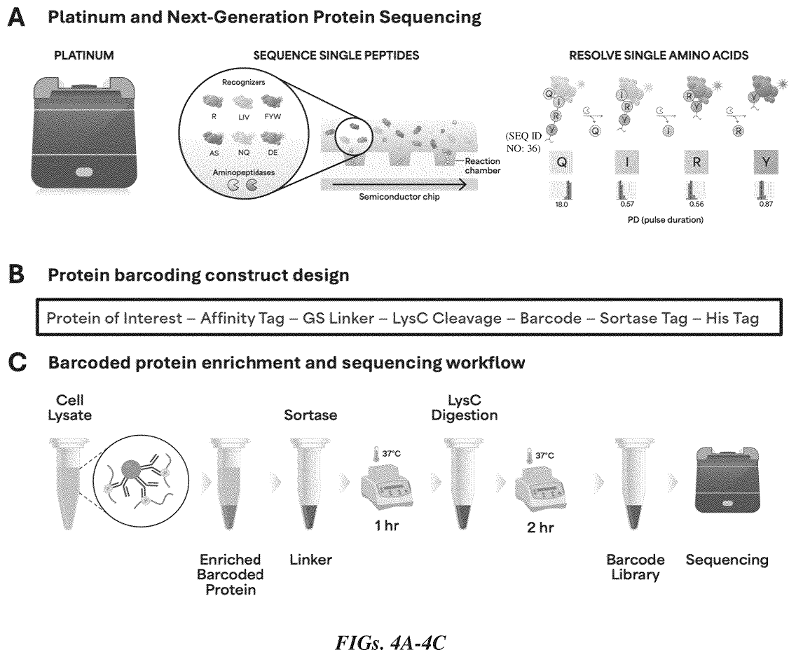

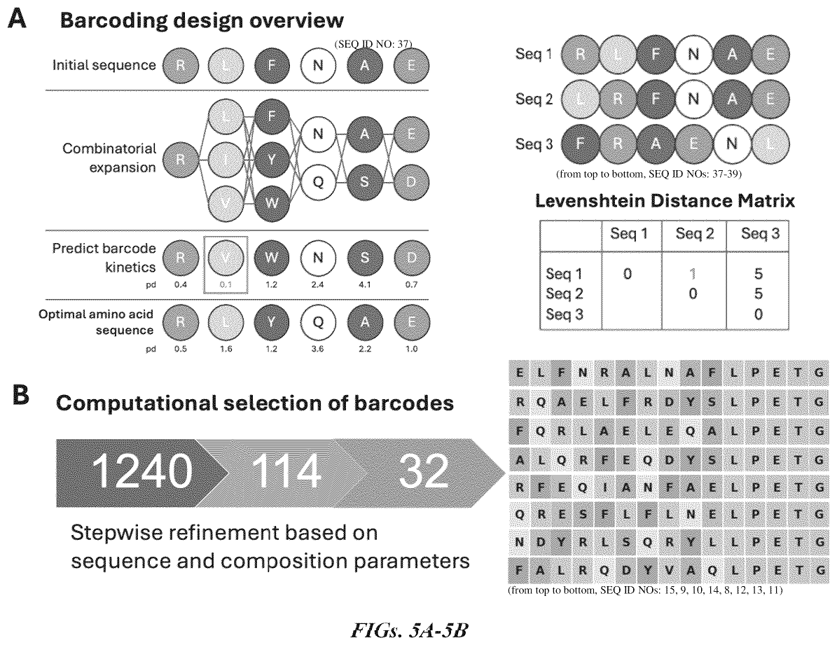

Recent innovations in single-molecule protein sequencing may offer a solution to these limitations. Novel protein sequencing technologies allow for the direct sequencing of protein barcodes with single-molecule resolution and an accessible benchtop workflow. NGPS involves the use of fluorescently tagged N-terminal amino acid (NAA) recognizer proteins to determine the order of amino acids in a peptide bound to a semiconductor chip (FIG. 4A). By distinguishing peptides based on their amino acid sequences rather than mass/charge ratios, NGPS overcomes some of the key challenges of mass spectrometry, such as the inability to resolve peptides with identical or highly similar amino acid compositions. This capability enables precise identification of protein sequences and opens the door to a range of new applications in protein characterization. In addition, the straightforward sample preparation and data analysis workflows make NGPS a highly accessible approach to protein barcode implementation.

The concept of protein barcoding is rooted in the success of DNA barcoding, a technique that has been widely applied in genomics and transcriptomics. DNA barcodes are short sequences of DNA that encode information and can be efficiently decoded using next-generation sequencing. This approach enables high-throughput analyses such as tracking sample identity in multiplexed libraries and mapping single-cell gene expression. However, while DNA barcodes have found broad use in molecular biology, their application to protein analysis has been more limited due to the need to retain a genotype-phenotype connection for readout, as well as the inability to directly detect successful translation with DNA barcodes.

One area where protein barcoding has shown particular promise is in the development of nucleic acid therapies. For instance, nucleic acid delivery systems, such as lipid nanoparticles (LNPs), often require tracking of both the uptake and functional delivery of therapeutic cargo to specific tissues or cells. While DNA barcodes have been used to track LNP uptake, they can fail to confirm the functional delivery and activity of the encoded proteins. Protein barcodes, on the other hand, can provide direct readouts of protein function and localization, offering a more precise and scalable method for tracking the success of nucleic acid delivery vectors.

In protein engineering, protein barcodes also hold significant potential. By tagging different variants of peptides with unique sequences, researchers can use barcoding to track the functional properties of engineered proteins in complex screening assays. This approach enables the rapid identification of proteins with desirable traits, such as improved stability, binding affinity, or enzymatic activity, which are critical for the development of new biotherapeutics.

In addition to gene therapy and protein engineering, protein barcoding has applications in other areas, such as studying protein-protein interactions, tracking protein subcellular localization, and even screening small-molecule libraries. The ability to encode functional information within peptides and decode it with high accuracy and resolution will enable researchers to gain deeper insights into complex cellular biology.

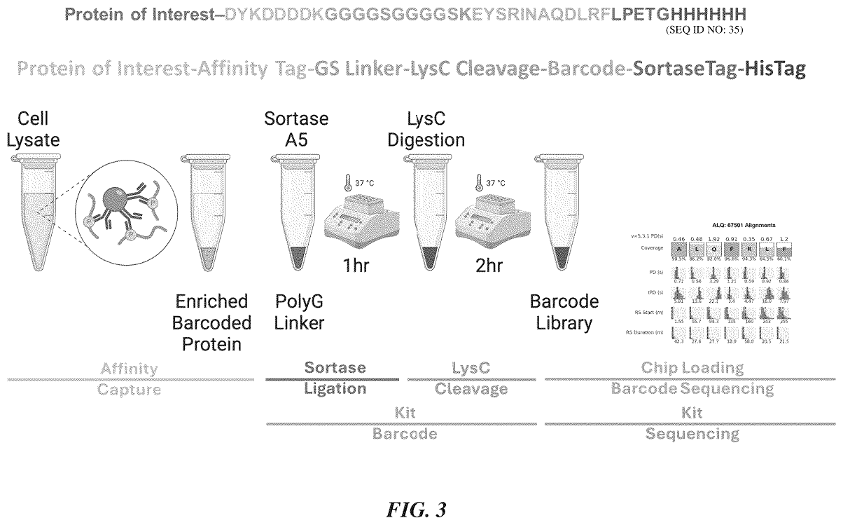

Accordingly, this Example relates to the development of a protein barcoding workflow combined with NGPS as a tool for advancing protein characterization with an accessible benchtop workflow. The results in this Example include an evaluation of key performance metrics, including dynamic range and limit of detection, in the context of an optimized set of eight barcodes. This Example serves as a foundation for the implementation of protein barcoding and NGPS workflows across a range of applications. For example, FIGS. 2A-2B show example workflows for functionalizing a barcode of a fusion polypeptide for surface immobilization by ligation of a polyglycine-streptavidin conjugate (“polyG-linker”). FIG. 2C shows example configurations of barcodes attached to N-terminal or C-terminal positions relative to a POI. FIG. 2D shows an example construct of a polyG-linker. FIG. 2E shows an example construct of a barcode functionalized with a polyG-linker. FIG. 3 shows an example workflow of barcode functionalization.

### Results

### Barcode Construct Design and Testing

As a first step in this study, expression constructs for barcoded proteins were designed and tested. To achieve efficient enrichment of barcoded protein expressed in cell or tissue, constructs were designed containing a FLAG tag and a unique barcode sequence, followed by a sortase tag with an optional 6xHisTag (FIG. 4B). The FLAG affinity tag was selected for several reasons: 1) it enables enrichment down to 15 fmol input from cell or tissue lysate; 2) it is easily accessible on the surface of the protein due to its charged residues and hydrophilic nature; 3) its smaller footprint reduces folding issues usually associated with larger affinity tags on smaller proteins; and 4) it can easily be cleaved by endopeptidase enterokinase (enteropeptidases), which recognizes DDDDK (SEQ ID NO: 7) of the FLAG affinity handle and digests C-terminally to K. A sortase tag was added as part of every barcode construct design to allow specific covalent modification to the barcode attached to the protein. Sortase A Pentamutant, an enzyme, is an engineered version of the wild-type sortase from Staphylococcus aureus that shows significantly higher activity than the wild-type sortase. Sortase belongs to a class of transpeptidases that utilize an active site cysteine thiol to modify proteins by recognizing and cleaving a carboxy-terminal sorting signal, LPXTG (SEQ ID NO: 2) (where X is any amino acid), between the threonine and glycine residues. A nucleophile-containing poly-glycine sequence, (Gly) n (where n=3 or more glycine residues), is used to attach a wide variety of labels such as peptides, DNA, carbohydrates, or fluorophores.

For the initial testing of this approach, the following barcoded proteins were generated and loaded on FLAG antibody beads: a synthetic peptide BC228, SARS-COV2-S1-RBD, and p53. Workflow A was followed as described in the Experimental Procedures section. The prepared libraries were then sequenced. These steps resulted in successful sequencing (data not shown); however, the sample input was 500 pmol and the overall reaction time was 2 days. To reduce the time and input requirements, a unique G-linker containing polyG as a nucleophile for a sortase-mediated ligation was designed and generated (FIGS. 4B-4C). Elimination of the DBCO click reactions from the K-Linker allowed the barcode to be directly attached and loaded onto the chip for sequencing. However, this introduces another issue, as the enterokinase has promiscuity with the G-linker, and it also has difficulty accessing the cleavage site while the FLAG antibody beads are bound to the FLAG tag on the barcoded protein. To eliminate these issues, a flexible GS Linker (GGGGSGGGGS (SEQ ID NO: 6)) was added between the affinity handle and barcode sequence (FIGS. 4B-4C). An additional amino acid, lysine (K), was also added between the spacer and N-terminus of the barcode sequence (e.g. BC265) to replace the enterokinase with LysC as a cleavage protease. LysC has no promiscuity with the G-linker, and LysC enzymatic cleavage separates the barcode from the FLAG-captured protein. The flexible GS Linker helps create a spacer for easy accessibility of affinity enrichment, allows flexible folding, and its hydrophilic nature helps keep the LysC cleavage site on the protein surface for easy accessibility.

The combination of these unique tags, including the barcode, comprises less than 35 amino acids in length, minimizing structural folding complications arising from larger and bulky tags. This modified workflow also enables faster enrichment of barcodes from cell lysate to sequencing. Overall, these design changes with the newly created G-linker workflow as shown in FIGS. 4B-4C resulted in unprecedented sensitivity, enabling a 10,000-fold reduction in sample input from 500 pmol down to 50 fmol (data not shown). Furthermore, the total time from cell lysate to loading on chip was reduced from two days to less than six hours, with less than one hour of hands-on time.

Following successful optimization of the workflow, the process was refined for computational generation of barcodes (FIGS. 5A-5B). The barcodes are a unique sequence of 10 to 12 amino acids that are optimized for NGPS. Over a thousand barcodes were generated, with each set containing 114 barcodes with equal sequencing capabilities, reduced bias, and low confusability between sequences, allowing random combination of any barcodes within a given pool (FIG. 5B). For initial validation, a set of eight peptide barcode sequences optimized for sequencing that reliably produce distinct sets of barcodes with minimal false discovery rates (FDR) was selected (see Experimental Procedures). These eight barcodes are shown in Table 1.

TABLE 1
Summary of normalization factors used for each
barcode (set of eight)

| | | Normali- |
|---|---|---|
| | | zation |
| Barcode | Sequence | factor |
| BC028 | RFEQIANFAELPETG (SEQ ID NO: 8) | 0.0939 |
| BC032 | RQAELFRDYSLPETG (SEQ ID NO: 9) | 0.1185 |
| BC049 | FORLAELEQALPETG (SEQ ID NO: 10) | 0.1424 |
| BC051 | FALRQDYVAQLPETG (SEQ ID NO: 11) | 0.0314 |
| BC067 | QRESFLFLNELPETG (SEQ ID NO: 12) | 0.1448 |
| BC075 | NDYRLSQRYLLPETG (SEQ ID NO: 13) | 0.1029 |
| BC079 | ALQRFEQDYSLPETG (SEQ ID NO: 14) | 0.0590 |
| BC096 | ELFNRALNAFLPETG (SEQ ID NO: 15) | 0.3070 |

Validation was further conducted on a set of 24 peptide barcode sequences. For this validation, a set of 24 peptide barcode sequences optimized for sequencing that reliably produce distinct sets of barcodes with minimal FDR was selected. These 24 barcodes are shown in Table 2.

TABLE 2
Summary of normalization factors for each barcode (set of 24)

| | | Barcode | Normalization |
|---|---|---|---|
| Barcode | Full Sequence | Length | Factor |
| BC028 | DYKDDDDKGGGGSGGGGSKRFEQIANFAELPETGH | 12 | 0.03166408 |
| | (SEQ ID NO: 16) | | |
| BC032 | DYKDDDDKGGGGSGGGGSKRQAELFRDYSLPETGH | 12 | 0.02810914 |
| | (SEQ ID NO: 17) | | |
| BC049 | DYKDDDDKGGGGSGGGGSKFQRLAELEQALPETGH | 12 | 0.01801997 |
| | (SEQ ID NO: 18) | | |
| BC051 | DYKDDDDKGGGGSGGGGSKFALRQDYVAQLPETGH | 12 | 0.00311264 |
| | (SEQ ID NO: 19) | | |
| BC067 | DYKDDDDKGGGGSGGGGSKQRESFLFLNELPETGH | 12 | 0.02070946 |
| | (SEQ ID NO: 20) | | |
| BC075 | DYKDDDDKGGGGSGGGGSKNDYRLSQRYLLPETGH | 12 | 0.10873504 |
| | (SEQ ID NO: 21) | | |
| BC079 | DYKDDDDKGGGGSGGGGSKALQRFEQDYSLPETGH | 12 | 0.02643672 |
| | (SEQ ID NO: 22) | | |
| BC096 | DYKDDDDKGGGGSGGGGSKELFNRALNAFLPETGH | 12 | 0.04544074 |
| | (SEQ ID NO: 23) | | |
| BC201 | DYKDDDDKGGGGSGGGGSKAIQLRDSERYNFLPETGH | 14 | 0.00850789 |
| | (SEQ ID NO: 54) | | |
| BC203 | DYKDDDDKGGGGSGGGGSKDARLYNRESIQFLPETGH | 14 | 0.01231011 |
| | (SEQ ID NO: 55) | | |
| BC208 | DYKDDDDKGGGGSGGGGSKEIQRADSYLRNFLPETGH | 14 | 0.01789448 |
| | (SEQ ID NO: 56) | | |
| BC210 | DYKDDDDKGGGGSGGGGSKELSIYARDNRQFLPETGH | 14 | 0.04695172 |
| | (SEQ ID NO: 57) | | |
| BC212 | DYKDDDDKGGGGSGGGGSKERQRDSYINLAFLPETGH | 14 | 0.04424083 |
| | (SEQ ID NO: 58) | | |
| BC213 | DYKDDDDKGGGGSGGGGSKERSRDLAYINFQLPETGH | 14 | 0.05097362 |
| | (SEQ ID NO: 59) | | |
| BC214 | DYKDDDDKGGGGSGGGGSKESLRANIYDRQFLPETGH | 14 | 0.06625727 |
| | (SEQ ID NO: 60) | | |
| BC216 | DYKDDDDKGGGGSGGGGSKEYSRINAQDLRFLPETGH | 14 | 0.0988586 |
| | (SEQ ID NO: 61) | | |
| BC218 | DYKDDDDKGGGGSGGGGSKINEQARDLSYRFLPETGH | 14 | 0.07794919 |
| | (SEQ ID NO: 62) | | |
| BC222 | DYKDDDDKGGGGSGGGGSKNAQYDRELSRIFLPETGH | 14 | 0.03786358 |
| | (SEQ ID NO: 63) | | |
| BC227 | DYKDDDDKGGGGSGGGGSKNYQRDLESIRAFLPETGH | 14 | 0.09816977 |
| | (SEQ ID NO: 64) | | |
| BC229 | DYKDDDDKGGGGSGGGGSKRDALQIERSYNFLPETGH | 14 | 0.0291754 |
| | (SEQ ID NO: 65) | | |
| BC231 | DYKDDDDKGGGGSGGGGSKRENAQSYDIRLFLPETGH | 14 | 0.05339564 |
| | (SEQ ID NO: 66) | | |
| BC233 | DYKDDDDKGGGGSGGGGSKRLEQRDSYINAFLPETGH | 14 | 0.03421943 |
| | (SEQ ID NO: 67) | | |
| BC234 | DYKDDDDKGGGGSGGGGSKRLEYSDRINAQFLPETGH | 14 | 0.00762962 |
| | (SEQ ID NO: 68) | | |
| BC236 | DYKDDDDKGGGGSGGGGSKRNRQESYLDAIFLPETGH | 14 | 0.03337505 |
| | (SEQ ID NO: 69) | | |

### Normalization of Barcodes in Mixtures

After selecting these barcodes, a set of normalization factors to increase linearity and reduce bias in multiplex mixtures was determined. All eight barcodes were mixed at equimolar concentration to produce 1:1 mixture of plexity of eight each at 3.125 pmol (62.5 nM), with total sample input of 25 pmol. The normalization factors were initially generated by performing over 25 sequencing runs, resulting in over 200 data points from 1:1 mix, 10-fold, and 100-fold dynamic range mixtures of eight barcodes (data not shown). Runs were repeated in triplicate and with loading at 33 μM, 100 μM, and 300 μM. To calculate the normalization factors, the raw alignments for each barcode on each run were divided by total alignments to generate raw observed fractions. These raw observed fractions were re-normalized by known expected fractions, resulting in a pre-normalization factor. The median pre-normalization factor was taken to re-normalize, generating final normalization factors as shown Table 1.

### Normalization and Reproducibility in 8-Barcode Mixtures

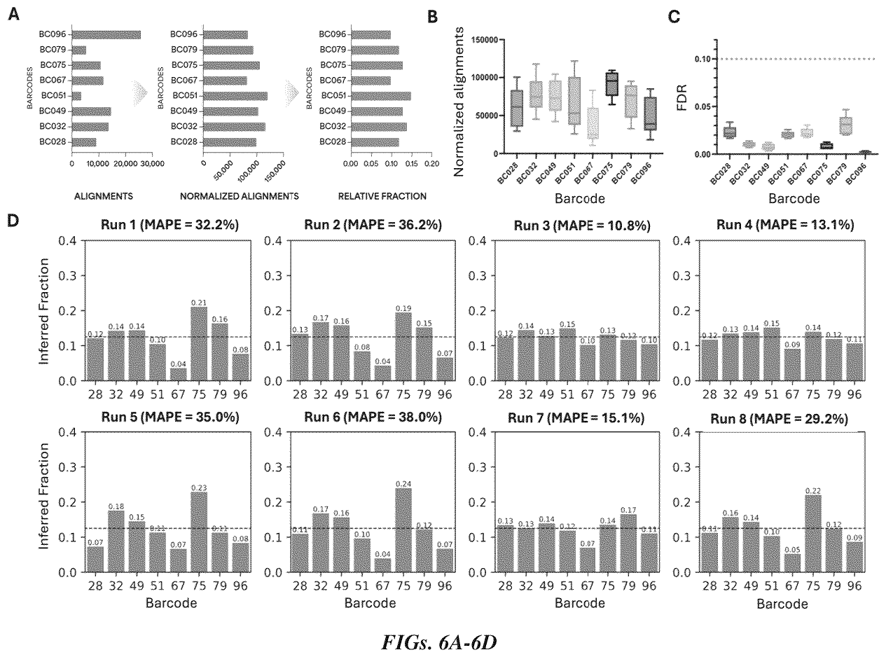

An additional eight runs of 1:1 equimolar mix of all eight barcodes at 25 pmol total sample input were performed, and the above established normalization factors were applied to extract relative abundance of each barcode. As shown in FIG. 6A, the alignments were converted to normalized alignments by dividing the normalization factor for each barcode, then each of the normalized alignments was divided by the sum of normalized alignments to extract the relative fraction of each observed barcode. The cumulative plot of normalized alignments for each barcode across eight runs is shown in FIG. 6B. All eight barcodes were successfully identified with an FDR below the 10% cutoff (FIG. 6C), and the relative abundance from each run showed ~25% MAPE (FIG. 6D), indicating high accuracy. These results establish the reproducible recovery of eight barcodes in expected ratios across multiple runs.

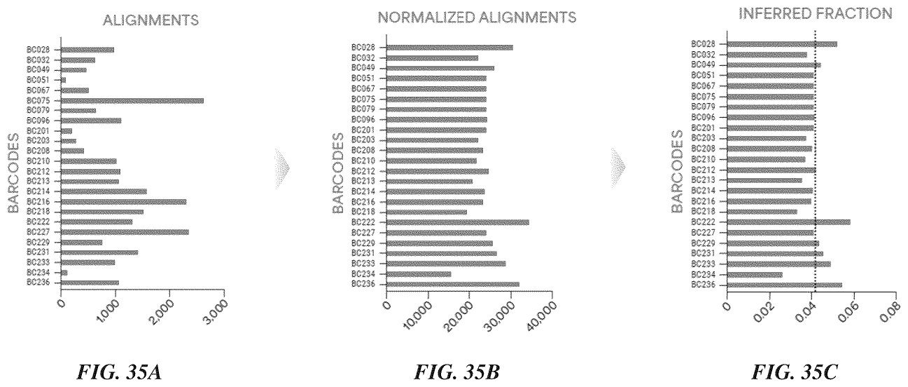

To further validate these results, additional runs of 1:1 equimolar mix of all 24 barcodes shown in Table 2 at 104 fmol samples input were performed, and the above established normalization factors were applied to extract relative abundance of each barcode. As shown in FIGS. 35A-35C, the alignments (FIG. 35A) were converted to normalized alignments (FIG. 35B) by dividing the normalization factor for each barcode, then each of the normalized alignments was divided by the sum of normalized alignments to extract the relative fraction of each observed barcode (FIG. 35C). All 24 barcodes were successfully identified with an FDR below the 10% cutoff (FIG. 35C). These results demonstrate that normalization allows for relative quantitation of barcodes, which is important for assessing protein expression, optimizing drug discovery, and evaluating protein trafficking and characteristics.

### Limit of Detection

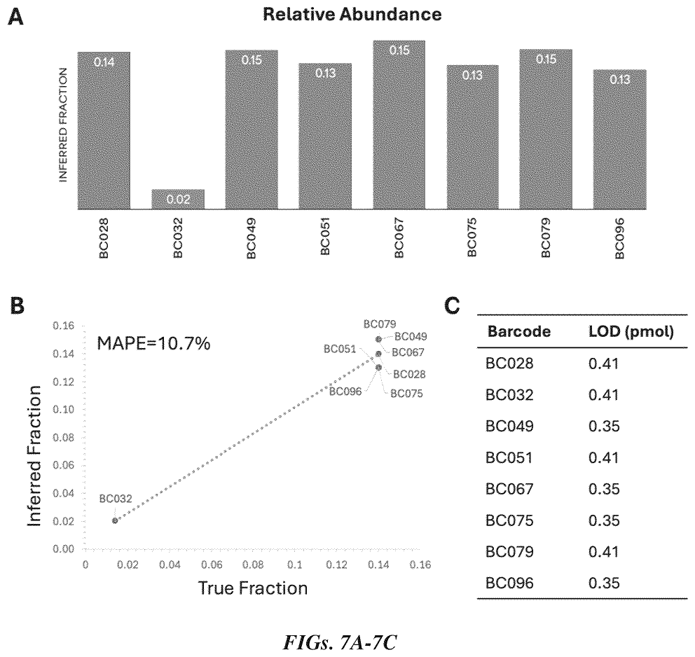

Next, the limit of detection (LOD) was tested at 25 pmol total input, where each barcode is either at 5 pmol or lower in a plexity of eight. In this experiment, seven barcodes were maintained at 1:1 equimolar mix (3.52 pmol each) and one barcode was varied by 10-fold lower (0.352 pmol) input concentration. Eight total runs were performed, varying one barcode per run to cover all eight barcodes at the lowest input of 352 fmol. Four barcodes were successfully recovered (defined as >20 alignments and <10% FDR) at the lowest concentration tested (BC049, BC067, BC075, BC096). However, four barcodes had higher than 10% FDR (BC028, BC032, BC051, BC079) when tested at the lowest input. The runs were repeated for these four barcodes, increasing the lowest input to 410 fmol. This resulted in successful identification of the four remaining barcodes at <10% FDR (BC028, BC032, BC051, BC079). FIG. 7A shows an example dataset for the run with BC032 at the lowest input, and FIG. 7B shows the true fraction plotted against the inferred fraction for this run, resulting in an MAPE of 10.7%. These results demonstrate the relative abundance recovered from the LOD experiment of barcode 32 at the lowest input correlates well with the expected fraction. Likewise, when plotting the expected barcode fraction against the inferred fraction across all eight LOD runs, the calculated cumulative MAPE was 30%. Therefore, the LOD for all eight barcodes were determined to be 410 fmol or below (FIG. 7C).

### Dynamic Range

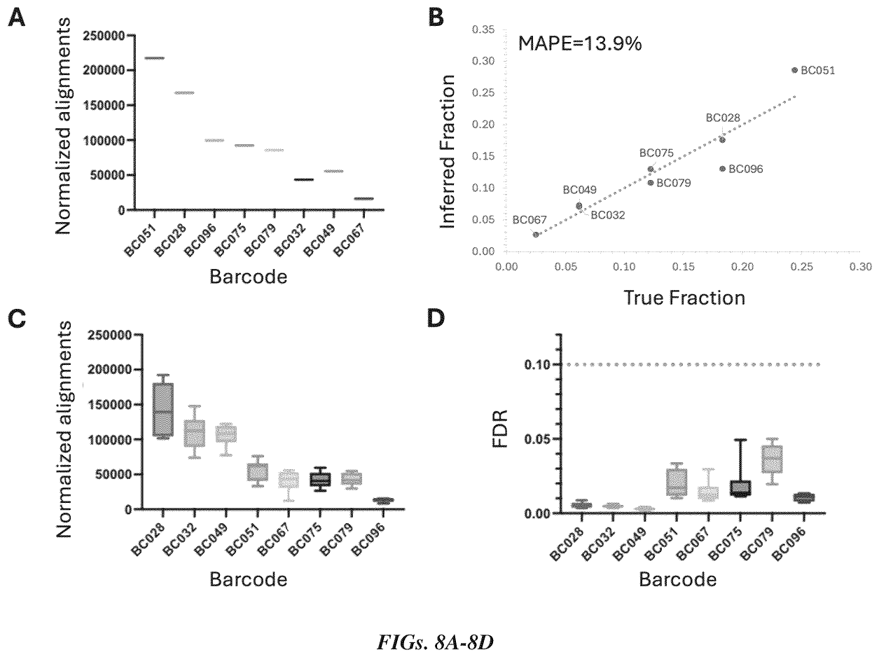

The dynamic range of barcode concentrations measurable within an eight-barcode mixture was evaluated. Barcodes were randomly mixed at 1× (BC051), 0.75× (BC028, and BC096), 0.5× (BC075, and BC079), 0.25× (BC032, and BC049), and 0.1× (BC067) to produce 10-fold dynamic ranges. As shown in FIG. 8A, all barcodes were identified with an FDR <10%, and the recovered relative abundance showed a good linear correlation after normalization, with an R2 of 0.9 and MAPE of 13.9% (FIG. 8B). Next, these ratios were scrambled within the same 10-fold dynamic range, and three different mixes with different barcodes at the 0.1× level (BC032, BC049, and BC075) were evaluated; all mixes with three repeats resulted in successful sequencing, with calculated MAPE of 24.3%, 15.9%, and 22.9%, respectively.

The reproducibility and robustness of this approach in recovering an unknown dilution within a 10-fold dynamic range was evaluated. Eight additional runs were performed for a 10-fold dynamic range with barcodes at 1× (BC028), 0.75× (BC032, and BC049), 0.5× (BC051, and BC067), 0.25× (BC075, and BC079), and 0.1× (BC096), as shown in FIG. 8C for the normalized alignments and FIG. 8D for the plots of FDR for each barcode. These results showed that all eight barcodes were successfully identified across all runs with FDRs <10%. In addition, the recovered relative abundance plotted against the true expected fraction from each run showed an MAPE of 21.5%. These results indicate that with an eight-plex barcode mixture with a total input of 25 pmole and the lowest concentration barcode at ~500 fmol, all eight barcodes are recovered across a 10-fold dynamic range that is still within the LOD. These results demonstrate the robustness of the assay and workflow across a wide range of relative abundances.

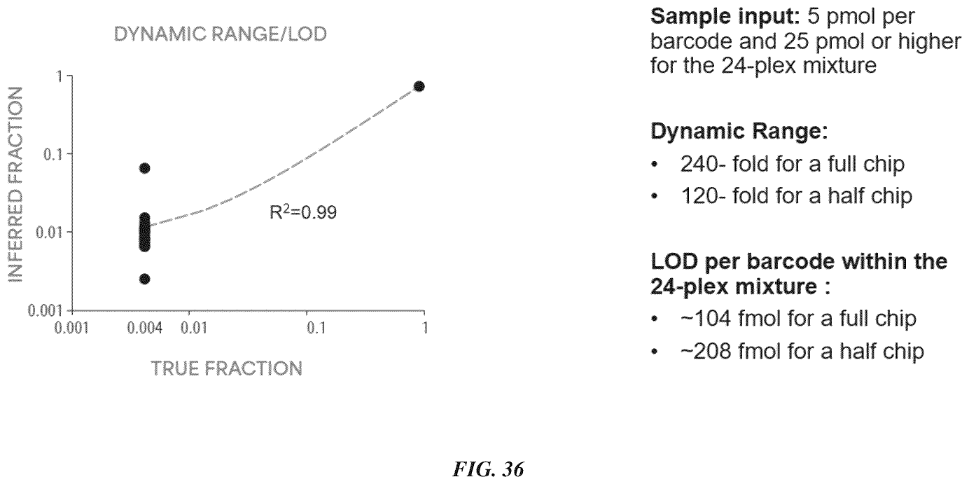

These results were further validated using the 24 barcodes shown in Table 2. A sample input of 5 pmol per barcode and 25 pmol or higher for the 24-plex mixture was used. FIG. 36 demonstrates that the dynamic range for a full chip is 240-fold and the dynamic range for a half chip is 120-fold. FIG. 36 also shows that the limit of detection (LOD) per barcode within the 24-plex mixture was ~104 fmol for a full chip and ~208 fmol for a half chip. These results demonstrate the reproducibility and robustness of the barcoding system across a wide range of relative abundances to enable differentiation between the highest- and lowest-abundance proteins.

### Performance on a Mixture of Five Proteins

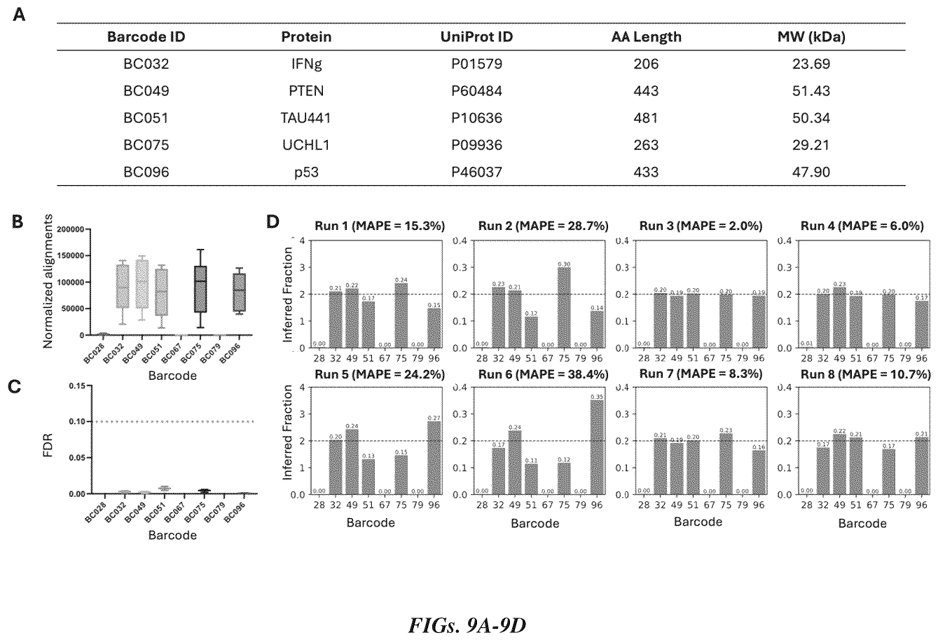

The performance of the barcoding workflow was evaluated in the context of full-length protein expression. Five barcoded protein constructs were generated, as shown in FIG. 9A. These five (IFNg-BC032, PTEN-BC049, TAU441-BC051, UCHL1-BC075, and p53-BC096) were all individually expressed and purified, and the purified barcoded proteins were mixed at 1:1 equimolar ratios (5 pmol per barcoded protein, for a total of 25 pmol) and subjected to the same purification and sequencing workflow as the synthetic barcodes. Eight libraries of this five-protein mix were prepared to test the robustness of assay across two lots of Barcoding Kit, two lots of sequencing kits, four lots of chips, four different sequencing instruments, and two operators. The normalized alignments for all eight runs of five-protein mixes show positive identification of all five barcodes (FIG. 9B), with FDR less than 10% (FIG. 9C). Across all eight runs, the MAPE ranged from 2.0% to 38.4%, with an average of 16.7% (FIG. 9D). These results demonstrate that the barcoding approach can accurately recover relative abundances in a mixture of full-length proteins.

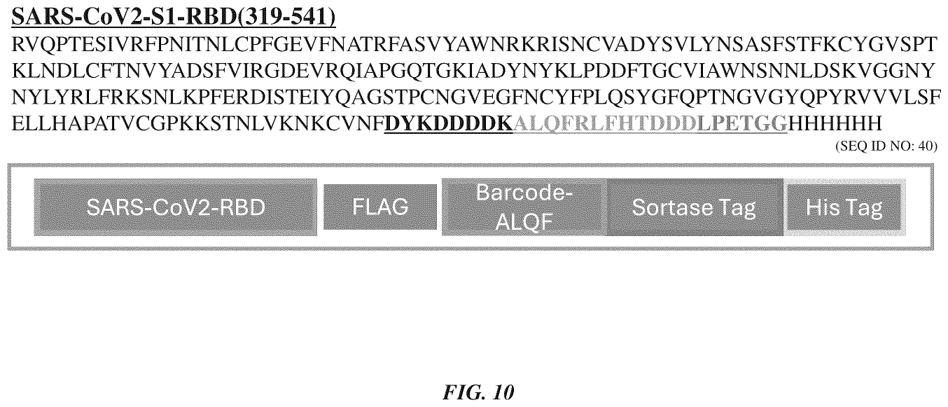

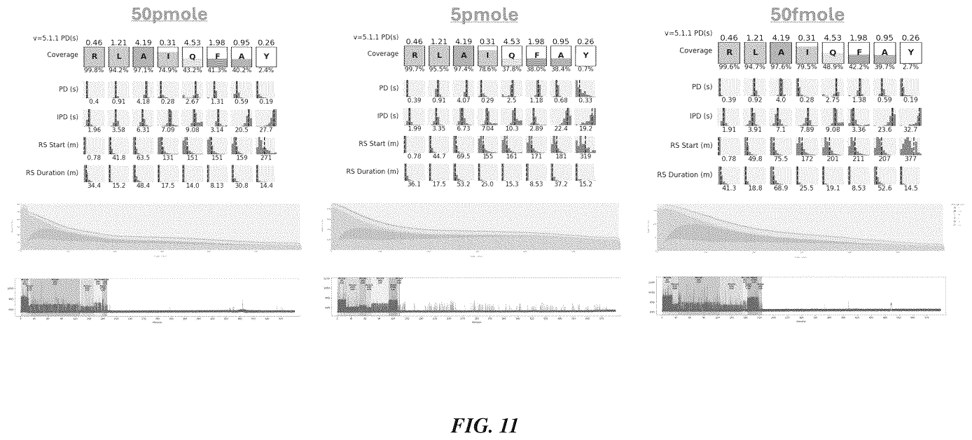

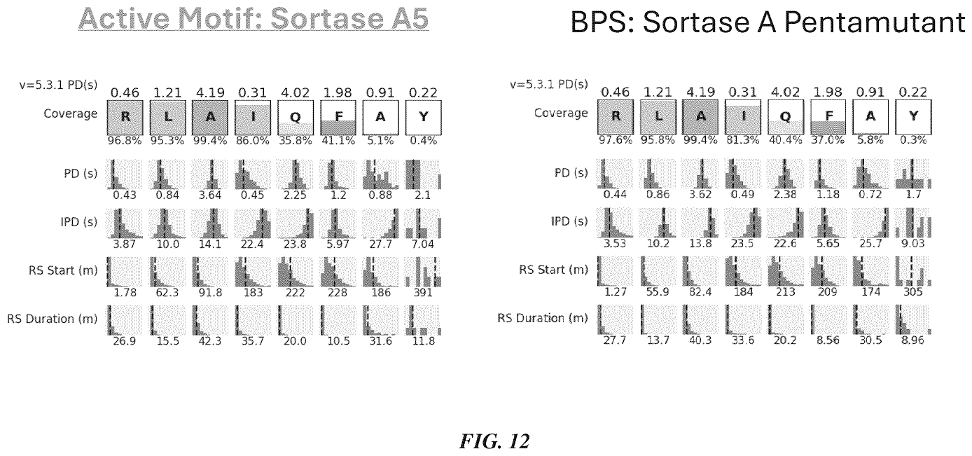

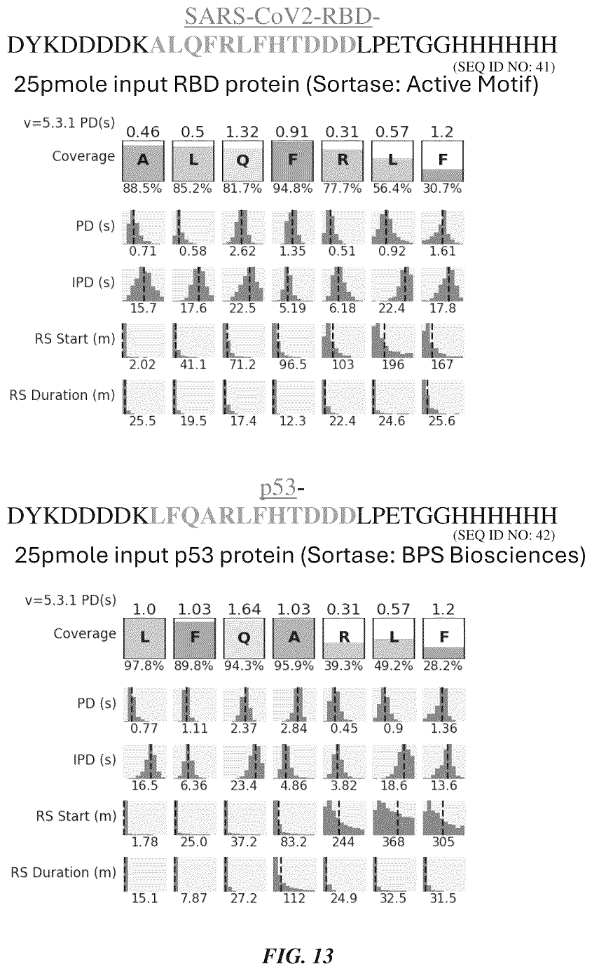

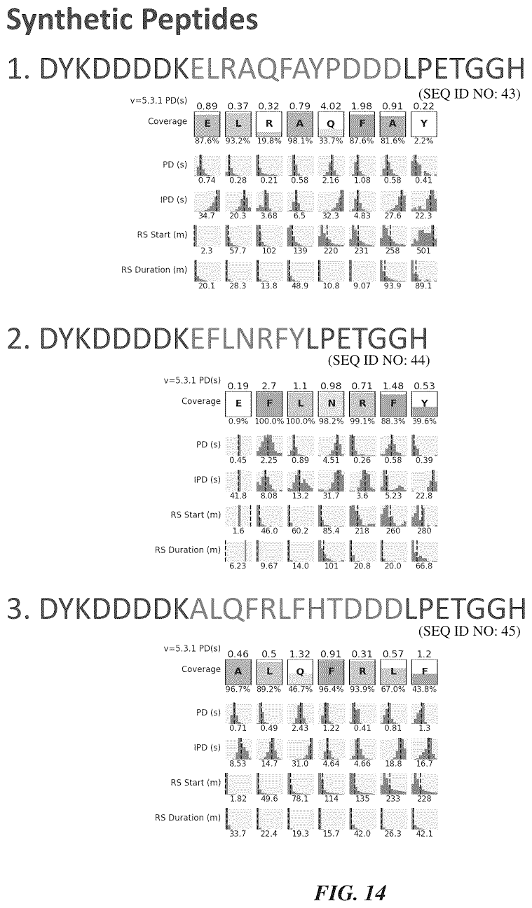

The present disclosure provides several examples of the use of the constructs described herein. For example, FIG. 10 shows an example construct of barcoded SARS-COV2-S1-RBS (319-541), FIG. 11 shows experimental results demonstrating significantly improved sensitivity with barcodes functionalized with polyG-linkers described herein, FIG. 12 shows experimental results demonstrating comparable activity of sortase A5 and sortase A Pentamutant (BPS Biosciences), FIG. 13 shows experimental results for SARS-COV2-RBS and p53 proteins prepared and analyzed according to the workflow of FIG. 3, And FIG. 14 shows experimental results for three model synthetic peptides analyzed in a 3-barcode mixture (25 pmol total with each barcode at 8.3 pmol, and 8.3 pmol total with each barcode at ~2.8 pmol).

This Example demonstrates the design and successful implementation of a set of barcode constructs for efficient protein labeling and subsequent protein sequencing. Overall, over 100 protein sequencing runs were conducted on over 50 chips, including 10 different lots of sequencing chips, 5 different lots of sequencing reagent kits, and 2 different barcoding kits, all producing an overall MAPE of 24.4% with 95% confidence interval (CI). Over a thousand barcode sequences were generated as part of this effort, with eight optimized peptide sequences chosen for subsequent validation. These barcodes were coupled with affinity tags, flexible linkers, LysC cleavage sites, and sortase tags to enhance barcode enrichment, reduce folding issues, and ensure effective isolation and labeling of proteins. Optimization of the expression construct design also reduced the sample input requirement by 10,000-fold (500 pmol to 50 fmol) and the hands-on time to less than one hour.

The analysis of barcode normalization and plexity showed successful sequencing and quantification across a 10-fold dynamic range, with relative abundances recovered with high accuracy (MAPE <25%) across multiple runs. In testing the limit of detection (LOD), barcodes as low as 352 fmol input were identifiable in an eight-plex mixture, and 50 fmol for single proteins. Additionally, when applying this system to a mixture of five proteins expressed in E. coli, all proteins were successfully identified with FDR <10% and a MAPE of 16.7%. These results validate the robustness, accuracy, and sensitivity of the barcoding system for multiplexed proteomics applications.

The ability to accurately normalize barcode abundance across a tenfold dynamic range and detect barcodes at low input quantities (down to 50 fmol) aligns with the need for sensitive, quantitative protein analysis in a variety of applications. However, to achieve successful recovery of relative abundance with high accuracy, it is critical to design experiments that balance the sample input, plexity, and dynamic range, all of which impact the LOD. The sample input directly correlates with the plexity and dynamic range, which then determines the relative fraction of barcodes from lowest to highest abundance. Several key factors can influence sample input, including host expression system, localization, and the target protein. An increase in plexity results in reduced dynamic range, which then requires increased sample input. For example, it was hypothesized that a 100-fold dynamic range could be achieved with increased sample input (e.g., 50 pmol or higher).

Protein barcoding with NGPS has the potential to overcome several limitations of traditional protein analysis methods, such as mass spectrometry and direct labeling. By leveraging the power of NGPS for single-molecule resolution, this approach enables precise detection and quantification of protein variants without the need for expensive equipment. In addition, the ability to monitor protein behavior and interactions with minimal disruption to native protein function (due to the compact size of the affinity/barcode tags) is particularly valuable in complex biological systems. These findings also support the growing role of protein barcoding in applications like nucleic acid therapy development, where direct tracking of protein delivery and function is essential. By ensuring high-fidelity protein sequencing with a broad dynamic range, this work demonstrates how protein barcoding, when paired with NGPS, offers a versatile, scalable, and accessible solution for advancing protein characterization and functional screening.

A person having ordinary skill in the art will understand that many different affinity tags beyond those described in this Example are known in the art and can be used according to the methods described herein. A person having ordinary skill in the art will also understand that any number of subsets of fusion polypeptides having affinity tags (e.g., 2, 3, 4, 5, 2-10, 5-50, or more subsets), and not only the specific subsets described in this Example, can be used according to the methods described herein. A person having ordinary skill in the art will also understand that a set of any number of unique barcode sequences (e.g., 2-100, 4-50, 10-30, 20-200, or more than 24 barcode sequences), and not only the specific barcode sequences described in this Example, can be used according to the methods described herein.

### Experimental Procedures

### Barcode Design and Optimization

To design barcodes compatible with a sequencing and analysis platform, an initial large set of candidate sequences was iteratively refined. First, recognizer-ordered sequences (ROS) were generated by assigning each amino acid recognizer a unique symbol (e.g., “1” for the Arginine Recognizer) and ensuring that all six recognizers in the V3 Sequencing Kit (FIG. 4A) were included, with no two consecutive recognizers being the same. These ROSs were then expanded into full amino acid barcode sequences by enumerating all valid residue substitutions for each recognizer, evaluating each candidate's predicted performance using a kinetic database of pulse durations, and discarding any prone to dropout (FIG. 5A).

To ensure reliability despite potential errors (e.g., missed or substituted residues), the Levenshtein distance between ROS was calculated and a minimum distance required between every pair. This ensured each barcode remained uniquely identifiable, even if partial errors occurred (FIG. 5A).

To compute error-resistant barcode sets, a heuristic approach was employed. An empty barcode set was created, and all candidate ROS were randomized and iterated over each ROS in this pool, extending the barcode set only if the new candidate met the edit-distance threshold. This process was repeated 1,000 times.

From the resulting population of candidate sets, one satisfying both size and composition criteria was selected (FIG. 5B). This yielded barcode sets with strong error tolerance and high confidence in their unique identification.

### Construct Design and Protein Purification

The following barcodes were designed and both 1) added to the full-length protein construct as well as 2) produced as synthetic barcodes:

```
  (SEQ ID NO: 16)
BC028, DYKDDDDKGGGGSGGGGSKRFEQIANFAELPETGH;
  (SEQ ID NO: 17)
BC032, DYKDDDDKGGGGSGGGGSKRQAELFRDYSLPETGH;
  (SEQ ID NO: 18)
BC049, DYKDDDDKGGGGSGGGGSKFQRLAELEQALPETGH;
  (SEQ ID NO: 19)
BC051, DYKDDDDKGGGGSGGGGSKFALRQDYVAQLPETGH;
  (SEQ ID NO: 20)
BC067, DYKDDDDKGGGGSGGGGSKQRESFLFLNELPETGH;
  (SEQ ID NO: 21)
BC075, DYKDDDDKGGGGSGGGGSKNDYRLSQRYLLPETGH;
  (SEQ ID NO: 22)
BC079, DYKDDDDKGGGGSGGGGSKALQRFEQDYSLPETGH;
  (SEQ ID NO: 23)
BC096, DYKDDDDKGGGGSGGGGSKELFNRALNAFLPETGH
```

The synthetic barcodes were custom synthesized by InnoPep (San Diego, CA), each supplied at 3 mg and with a purity greater than 95%. All synthetic peptides featured an N-terminal H and C-terminal carboxylic acid block NH2. They were initially reconstituted in DMSO to a concentration of 10 mM and stored at −20° C. until ready for the barcoding kit workflow. The peptides then go through the same sample preparation steps as the purified protein (below and FIG. 4C).

The five full-length proteins (IFNg-BC032, PTEN-BC049, TAU441-BC051, UCHL1-BC075, and p53-BC096) were cloned in pET21 (a) with (i) c-terminal FLAG tag for affinity purification, (ii) a flexible GS linker as a spacer between affinity tag and barcode, (iii) LysC-cleavage site, (iv) peptide barcode, (v) sortase tag, and an optional (vi) 6× His-tag. See FIG. 4B for an overview of the final construct design. All vectors were transformed into E. coli strain BL21 (DE3) (Genscript, New Jersey, USA) to express in Super Broth Auto-Induction Media (Grisp Research Solutions, Portugal) at 37° C., then transferred into 18° C. for overnight shaking at 200 RPM. The purification was done with anti-FLAG antibody magnetic beads to selectively capture FLAG-tagged, barcoded proteins of interest using either Pierce™ Anti-DYKDDDDK (SEQ ID NO: 24) Magnetic Agarose (ThermoFisher; Cat. No. A36797) or Anti-FLAG® M2 Magnetic Beads (MilliporeSigma; Cat. No. M8823). The optional primary or secondary purification was done using cobalt-based IMAC Talon Superflow (Cytiva, USA) resin. Enriched protein was buffer exchanged in 50 mM Tris-HCL pH 7.5, and 150 mM NaCl to be compatible with sortase reactions, and the concentration of each protein was quantified using A280 Nanodrop Spectrophotometer (Thermo Fisher Scientific).

Additionally, for the initial study (See Workflow section below) a synthetic peptide (BC265, DYKDDDDKGGGGSGGGGSKALQFRLFHTDDDLPETGH (SEQ ID NO: 25)) was designed in addition to a version that lacked the GS linker and the lysine cleavage site between the GS linker and barcode (BC228, DYKDDDDKALQFRLFHTDDDLPETGH (SEQ ID NO: 26)). Two protein constructs were also designed: SARS-COV2-S1-RBD domain (R319-F541) protein with FLAG tag, barcode sequence (ALQFRLFHTDDD (SEQ ID NO: 27)), sortase tag, and optional 6xHis-tag was cloned into pcDNA 3.1 vector and expressed in HEK293 as a secreted protein. The full-length p53 protein with FLAG tag, barcode sequence (LFQARLFHTDDD (SEQ ID NO: 28)), sortase tag, and optional 6xHis-tag (SEQ ID NO: 4) was cloned into pET21 and expressed in E. coli by BPS Biosciences (San Diego, CA).

### G-Linker Production

A peptide-DNA-streptavidin conjugate was used as the linker to position barcode peptides on the chip surface. A DNA duplex was used as the structural scaffold to keep peptides away from the surface matrix. A fluorescent dye was conjugated to one end of the DNA with an amino modifier near the Streptavidin for loading quantification. The other end of DNA was modified with an 02′-propargyl adenosine as the conjugation handle for an aspartate-rich peptide spacer. The N-terminus of the aspartate-rich peptide is modified with a polyG moiety as the sortase conjugation handle. The identity of the polyG-peptide-DNA-streptavidin conjugate (G-linker) was confirmed by SEC-MS on an Agilent QTOF system.

### Workflow (Enrichment, Ligation, Cleavage) Development)

Two different workflows were carried out through the course of the study. In the first version, Workflow A, the protein was enriched via affinity tag using anti-FLAG antibody magnetic beads at a minimum sample input of 500 pmol. The sortase ligation reaction was then performed with Picolyl-Azide-Gly-Gly-Gly (Vector labs, USA) at 37° C. for 1 hr; this reaction results in covalent attachment of barcoded protein or peptides to an azide handle. After washing away excess Gly-Gly-Gly-Picolyl-Azide, K-Linker (Quantum-Si, USA) was added. The barcode-ligated azide handle and DBCO moiety on the K-Linker were covalently attached via Strain-Promoted Alkyne-Azide Cycloaddition (SPAAC) click reaction at 37° C. for 16 hours, then the excess K-Linker was washed away. Finally, barcode linked K-Linker was cleaved from protein using enterokinase (Invitrogen, USA) or LysC enzymes (Quantum-Si, USA) at 37° C. for 2 hours or longer. The prepared barcode libraries were then loaded and sequenced on a sequencing instrument.

In the second version, Workflow B (FIG. 4C), the protein was enriched via affinity tag using anti-FLAG antibody magnetic beads at a sample input of 50 fmol or higher. The enriched sample was then incubated with 100 nM G-linker and 2 μM Sortase A5 enzyme in sortase reaction buffer (50 mM Tris-HCL pH 7.5, 150 mM NaCl, and 5 mM CaCl₂)) at 37° C. for 1 hr on thermomixer at 1000 RPM. This reaction results in covalent attachment of the G-linker to the barcoded protein, eliminating the need for click reactions from workflow A and reducing the required sample input 10,000-fold. Finally, the G-linker ligated barcode was cleaved from protein using LysC enzyme at 37° C. for 2 hours on thermomixer at 1000 RPM. This step releases the barcode-ligated G-linker from the FLAG-enriched protein of interest or peptides still bound on beads. The G-linker allows direct and stable anchoring of barcodes to the semiconductor chip surface. The ligated barcode libraries were stored at −20° C. until sequencing.

### Barcode Sequencing

The sequencing of the barcodes was carried out on a sequencing instrument according to the manufacturer's instructions. Briefly, approximately 100 μM of the barcoded G-linker was loaded, followed by the removal of excess, unbound barcodes. All sequencing was performed with the Sequencing Kit V3, which includes N-terminal amino acid (NAA) recognizers for 13 of the 20 canonical amino acids. Specifically, the kit contains a set of six NAA recognizers for LIV, FYW, and R, as previously described, along with additional recognizers for AS, DE, and NQ (FIG. 4A). The binding and dissociation of these NAA recognizers to the immobilized peptide barcodes are monitored in real time as individual on-off events. NAAs from immobilized peptides are sequentially cleaved by aminopeptidases, allowing the next amino acid to be exposed for NAA recognizers to bind (FIG. 4A). This process is repeated throughout the 10-hour run time.

### Data Analysis

The sequencing instrument produces pulse calls as output of the raw sequencing data during real-time data collection. The pulse calls were transferred to analysis software. Initially, all runs were analyzed using the Primary Analysis v2.8.0, which produces recognition segments of detected regions of interest at the aperture level. Then all runs go through secondary analysis using the Peptide Alignment v2.9.0, which takes primary analysis as an input and aligns observed recognition segments to the barcode reference at the aperture level. The resulting aperture-level results are filtered with a threshold score of 4.0 or above, then False Discovery Rate (FDR) is calculated with 20 decoy peptides and a reverse sequence of the reference. In general, an FDR of 10% or lower is required for a positive identification of barcodes. The number of apertures that pass strict filtering, FDR, and alignment are all grouped per barcode to plot the total number of alignments per run and total number of alignments for each barcode. Mean FDR is also calculated per identified barcode.

### Mean Absolute Percent Error

Mean Absolute Percent Error (MAPE) was computed for each experiment. For each barcode, percent error was computed by taking the absolute value of the predicted fraction minus the known fraction in the sample, and that result was divided by the true fraction. The mean of individual barcode percent errors across all samples is reported as the MAPE.

$$M A P E = 100 \frac{1}{n} {\sum }_{t = 1}^{n} \vert \frac{{A}_{t} - {F}_{t}}{{A}_{t}} \vert$$

### Example 2. Barcode Plexity Expansion

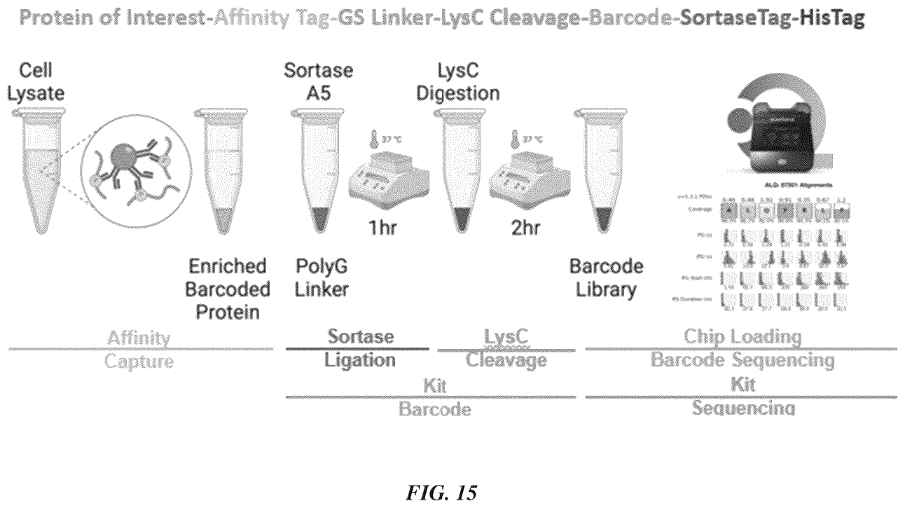

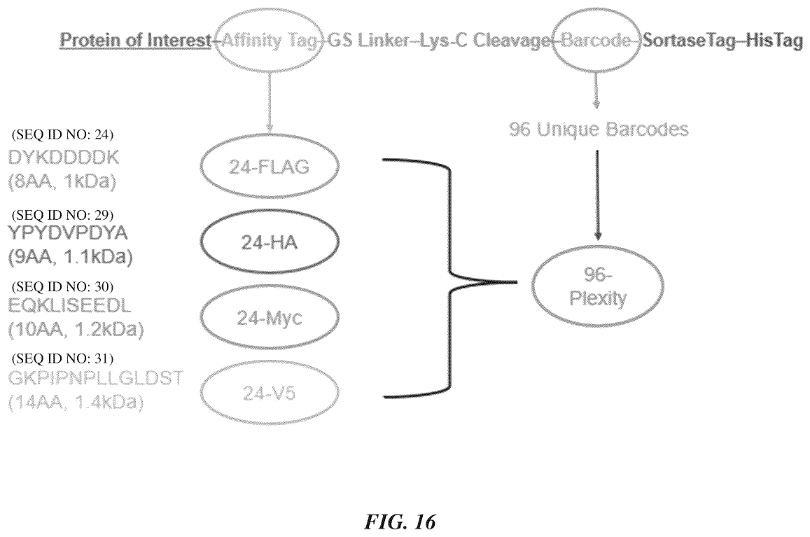

This Example relates to the development of a barcode workflow with expanded plexity that does not rely on the use of an increased number of peptide barcodes. The inventors of the present disclosure made the surprising discovery that the barcode construct described elsewhere herein and shown in FIG. 15 can be used to increase barcode plexity to 96 using only 24 peptide barcode sequences. To accomplish this, four different affinity tags were used with the same 24 peptide barcodes (FIG. 16). For example, 24 peptide barcodes were used with FLAG-tag (DYKDDDDK (SEQ ID NO: 24) (8AA, 1 kDa)), the same 24 peptide barcodes were used with HA-tag (YPYDVPDYA (SEQ ID NO: 29) (9AA, 1.1 kDa)), the same 24 peptide barcodes were used with myc-tag (EQKLISEEDL (SEQ ID NO: 30) (10AA, 1.2 kDa)), and the same 24 peptide barcodes were used with V5-tag (GKPIPNPLLGLDST (SEQ ID NO: 31) (14AA, 1.4 kDa)). The same sample can be processed using multiple affinity enrichment steps and separate sequencing runs. This approach allows for 96 barcode plexity while optimizing only 24 peptide barcodes.

### Example 3. Functionalization of POI with Luminescent Label

This Examples relates to the use of next generation protein sequencing (NGPS) technologies to sequence proteins and peptides at single-molecule resolution. Unlike conventional methods, such as Edman degradation or mass spectrometry (MS), NGPS can identify and quantify peptides in highly complex environments without the need for extensive fractionation or labeling.

Current NGPS platforms utilize a variety of approaches, including single-molecule sequencing, where fluorescently labeled recognizers are used to bind to N-term amino acids and are sequentially detected as peptides are immobilized and cleaved. These approaches offer unprecedented sensitivity, enabling the detection of low-abundance peptides, post-translational modifications (PTMs), and rare sequence variants-all crucial for screening therapeutic candidates.

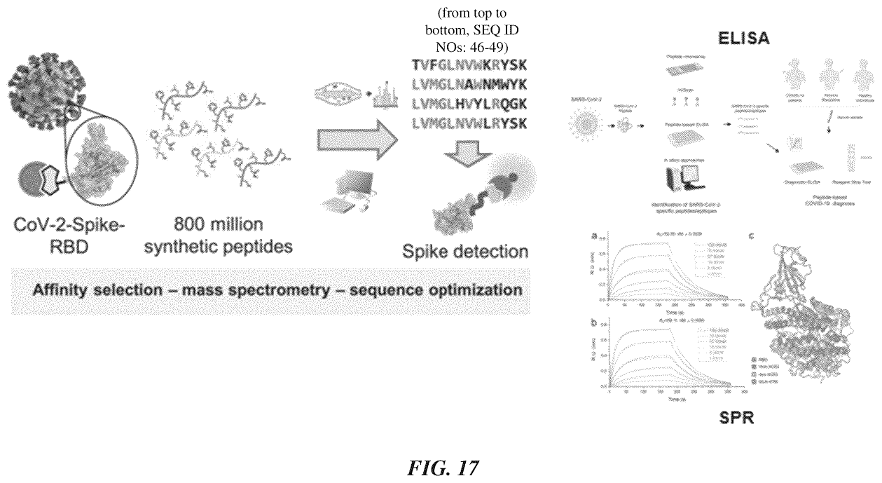

Traditional approaches include Surface Plasmon Resonance (SPR) and Digital ELISA. SPR quantitatively assesses binding kinetics and affinity by detecting mass changes on a sensor surface. SPR is a powerful tool for studying biomolecular interactions in real-time and provides detailed kinetic information. Digital ELISA, on the other hand, is a highly sensitive, endpoint detection method that focuses on counting individual molecules, offering single-molecule resolution and high sensitivity. These traditional approaches such as SPR may not be as sensitive as NGPS for very low concentrations and the cost of the instrument can be higher. The digital ELISA has single molecule sensitivity, however, it only provides a final measurement of the number of target molecules, rather than real time information and requires complex protocol and specialized instruments with higher cost. FIG. 17 shows examples of therapeutic peptide screening using traditional approaches.

### Peptide Library Generation and Screening in Drug Discovery

The screening process of the present disclosure begins with the generation of large, diverse peptide libraries, often via phage display, mRNA display, or synthetic combinatorial chemistry. These libraries can contain billions of unique sequences, representing an immense functional space.

Traditional screening platforms involve iterative rounds of selection, binding assays, and subsequent MS or DNA-based decoding. While powerful, these workflows are limited by throughput bottlenecks and potential biases during amplification or recovery. The advent of NGPS circumvents these limitations by enabling: direct sequencing of peptide candidates after selection, without the need for genetic encoding or amplification, high-throughput analysis of sequence-function relationships in complex mixtures, and parallel quantification and identification of peptides, including those with post-translational modifications.

### Pro Mode: An NGPS Enabled Peptide Screening Method

The Pro Mode screening process combines the powerful massively parallel single molecule NGPS with traditional capabilities of measuring the binding on and off rate kinetics in real time monitoring with high sensitivity.

### Library Preparation and Selection

Large peptide libraries are exposed to the target of interest (e.g., disease-relevant proteins, cell surfaces, or receptors). Peptides demonstrating affinity or functional activity are isolated, typically via affinity capture or cell sorting.

### Sample Processing for NGPS

Following selection, biological samples go through a library prep process briefly, proteins are digested into peptides and peptides are functionalized and prepared for NGPS analysis. This involves: immobilization onto semiconductor chip surfaces for single-molecule detection and minimal sample processing to preserve modifications and sequence integrity.

### Pro Mode Dye Labeling Kit

### Custom Binder Design

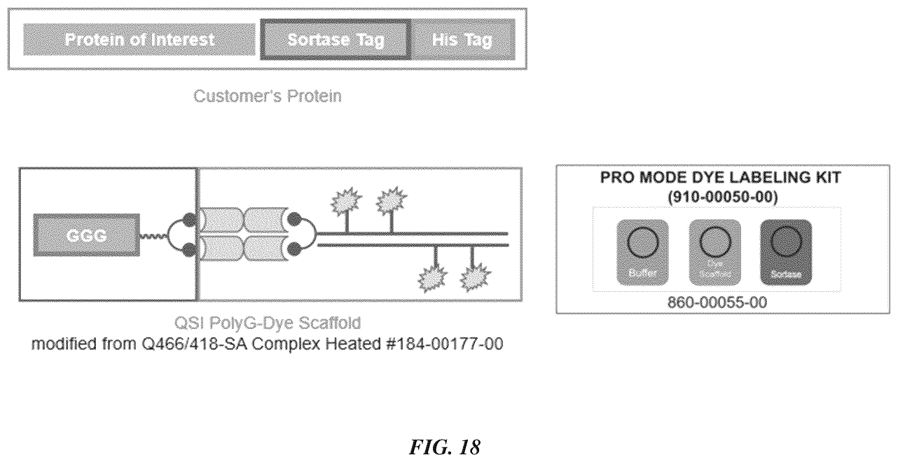

The custom binders are designed either with Proteins, enzymes, nanobody, or antibody constructs with a custom and short amino acid tag inserted into protein of interest to retain structural integrity and to site selectively label with dye scaffolds (FIG. 18).

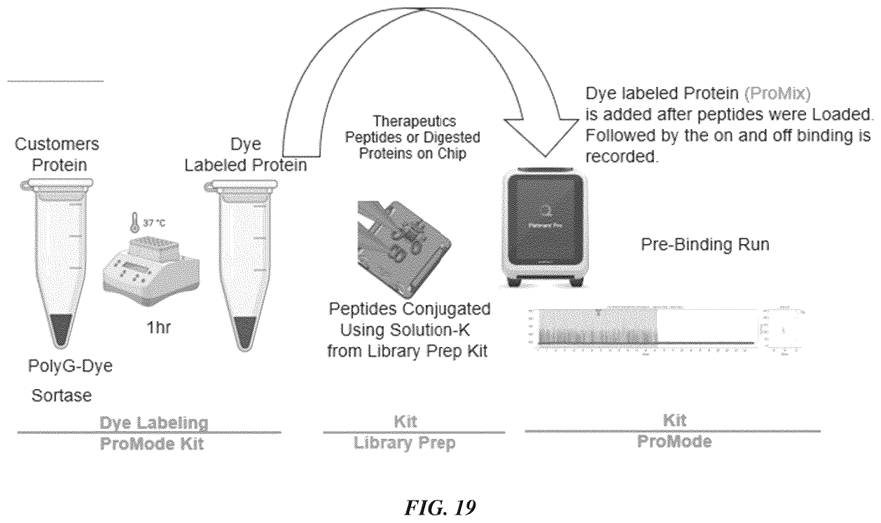

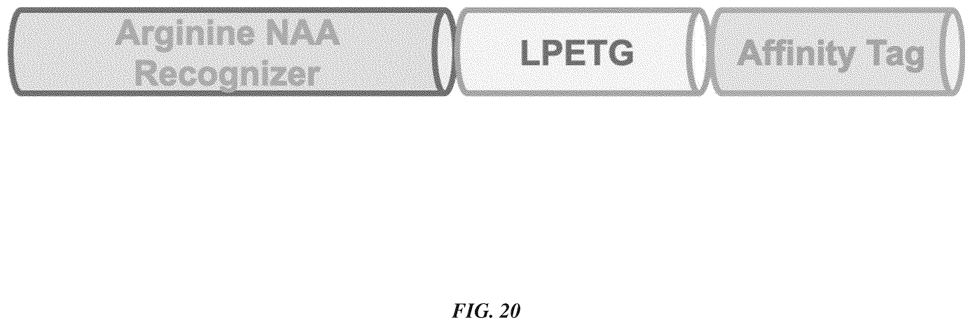

Proteins are purified, then the purified proteins are labeled using the dye labeling kit described herein and prepared for NGPS analysis (FIG. 19). An N-terminal amino acid (NAA) recognizer of Arginine was used as a model system to label and demonstrate the Pro Mode workflow (FIG. 20).

### Sortase Mediated Ligation

The R-recognizer with engineered LPETG (SEQ ID NO: 3) sorttag was expressed in E. coli and purified with his-tag affinity purification. The purified protein was labeled using Pro Mode Kit as highlighted in the Pro Mode protocol. Briefly, the purified LPETG (SEQ ID NO: 3) containing recognizer was incubated at 37 C for 1 hr in TBS containing calcium chloride with 4× molar concentration of Quantum-Si's Dye scaffold in presence of Sortase enzyme. Sortase A Pentamutant, an enzyme, is an engineered version of the wild-type sortase from Staphylococcus aureus that shows significantly higher activity than the wild-type sortase. Sortase belongs to a class of transpeptidases that utilize an active site cysteine thiol to modify proteins by recognizing and cleaving a carboxy-terminal sorting signal, LPXTG (SEQ ID NO: 2) (where X is any amino acid), between the threonine and glycine residues. A nucleophile-containing poly-glycine sequence, (Gly) n (where n=3 or more glycine residues), is used to attach a wide variety of labels such as peptides, DNA, carbohydrates, or fluorophores.

### HPLC Purification

The labeled R-recognizer reaction was then purified using HPLC system to generate pure singly labeled recognizer from unlabeled recognizer and also to separate from remaining excess of dye scaffold. (The full chromatogram is shown below, the peak at 11.05 min corresponds to R-recognizer labeled with dye scaffold, the remaining unreacted excess of free dye scaffold is eluted at 11.87 min)—no need to have spectrum. The fractions corresponding to labeled recognizer were collected and the collected fraction was concentrated using spin concentrator and ready to use on chip as new custom biner to screen peptides/protein targets.

### Pro Mode Data Acquisition Pre-Binding and Sequencing

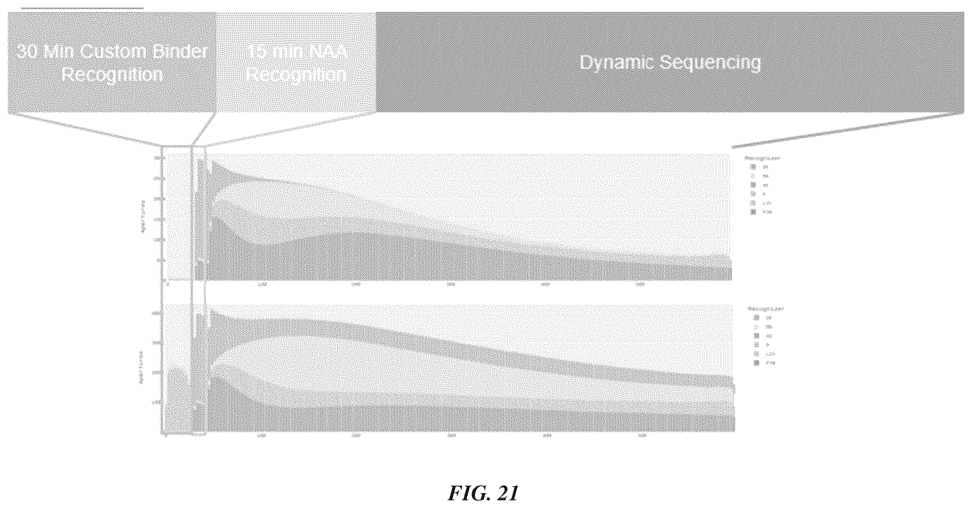

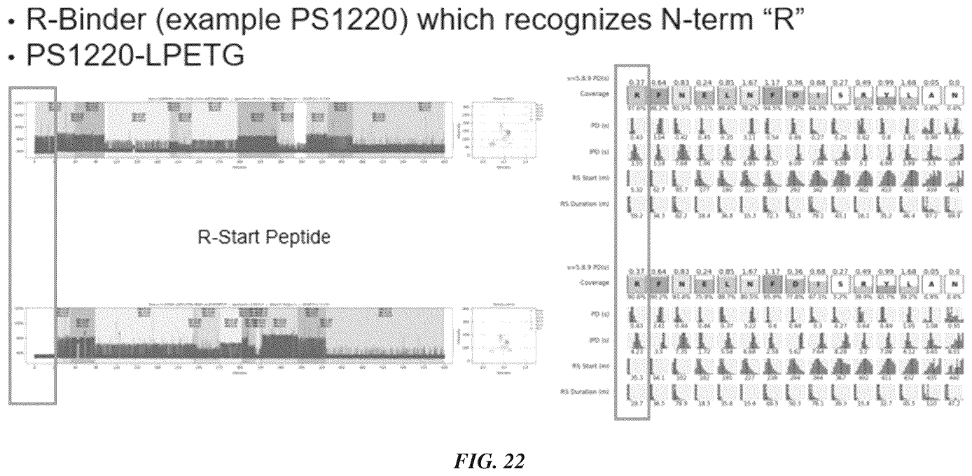

The prepared peptides are subjected to NGPS. The sequencing platform directly reads the amino acid sequences of millions of individual peptides. The real-time data streams enable instantaneous sequence acquisition. To test the newly labeled R-recognizer, a library of recombinant CDNF protein was prepared by following a standard library preparation protocol: the full-length protein was reduced, alkylated and digested using LysC enzyme to generate peptides with lysine truncation at C-term, the digested peptides were then functionalized using diazo transfer to convert lysine to azido lysine, and the modified peptides were clicked with DBCO containing K-linker to anchor the peptides on the semiconductor chip with 2 million apertures. The loaded and washed chip was ready to screen the peptides which have affinity to the custom binder; this selection process was carried out for 30 min pre-sequencing to record affinity driven pulses coming from the custom binder which contains reporter dye (FIG. 21). The digested peptides from CDNF protein do not contain any NAA arginine (R) and thus should not produce any pulsing activities for the first 30 min (FIG. 22). However, a specific NAA arginine containing synthetic peptide was spiked into CDNF library which resulted in an affinity driven on and off pulsing activities. The binding affinity extracted from the pulses was in alignment with an R binder present in the sequencing kit. The results demonstrated that the new sortase based labeling chemistry works well and can be used for any custom protein or antibody.

### Data Analysis and Candidate Identification

Sophisticated bioinformatics pipelines analyze the raw sequence data, mapping peptide identities, modifications, and abundance. Functional data and binding affinity can be correlated with sequence information to rapidly pinpoint high-value candidates for further development.

### Binding Constant

To extract the binding affinities, the dissociation constant can be calculated by taking PD and IPD and with the known concentration of custom binder one can extract the binding constat, as shown in the formula below.

$${k}_{off} = \frac{1}{mean PD} \\ {k}_{o n} = \frac{1}{( [ Ligand ] * mean IPD )} \\ \\ {K}_{D} = \frac{{k}_{off}}{{k}_{o n}}$$

### Validation and Downstream Applications

Promising peptide sequences identified via NGPS screening can be synthesized and validated in secondary assays ranging from in vitro binding studies to in vivo efficacy models. NGPS data also informs optimization cycles, guiding rational design, and structure and function relationship studies.

### Advantages of NGPS in Peptide Therapeutics Discovery

The integration of NGPS into peptide screening offers multiple advantages over conventional methodologies:
Throughput: Sequence thousands of peptides in a single experiment, dramatically accelerating hit identification;
Sensitivity: Detect low-abundance or rare peptides that might be missed in traditional screens;
Resolution: Directly identify post-translational modifications, and sequence variants;
Unbiased Discovery: Eliminate biases introduced by genetic encoding or amplification steps; and
Quantitative: Simultaneous quantification of peptide abundance and sequence relationships.

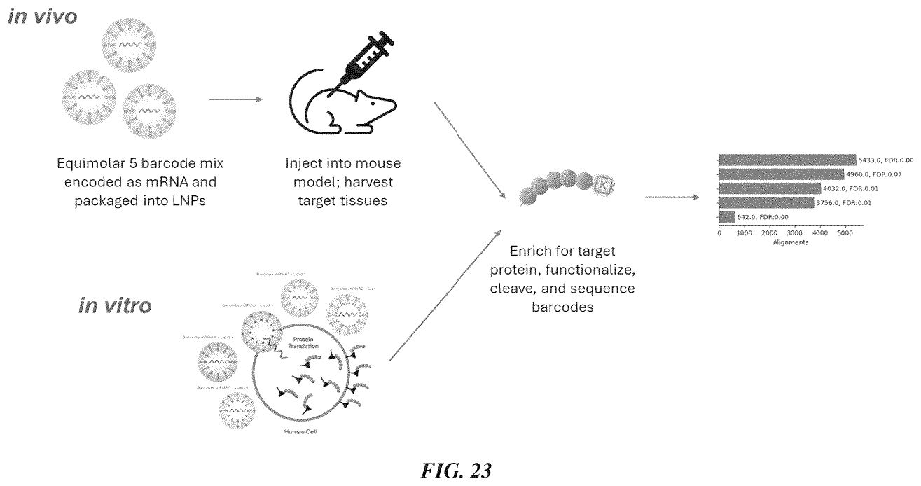

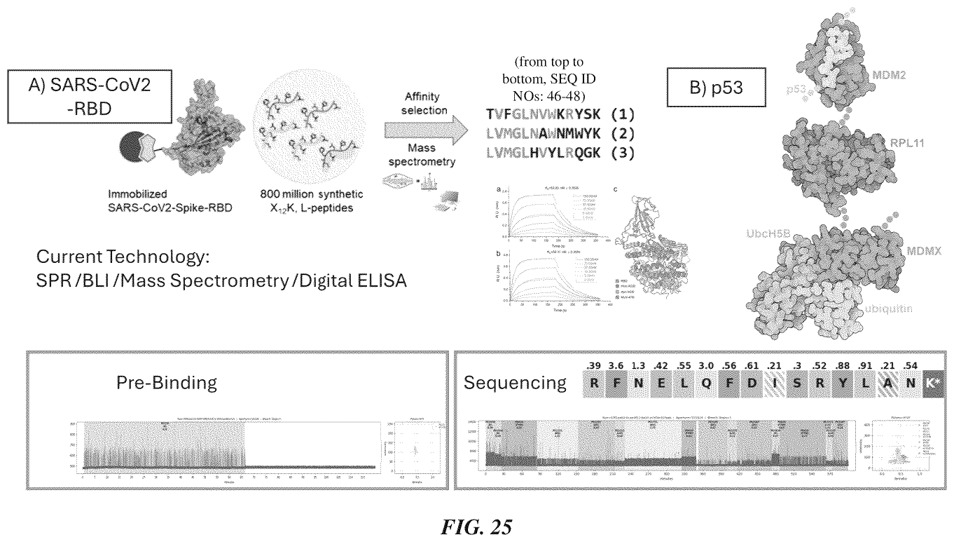

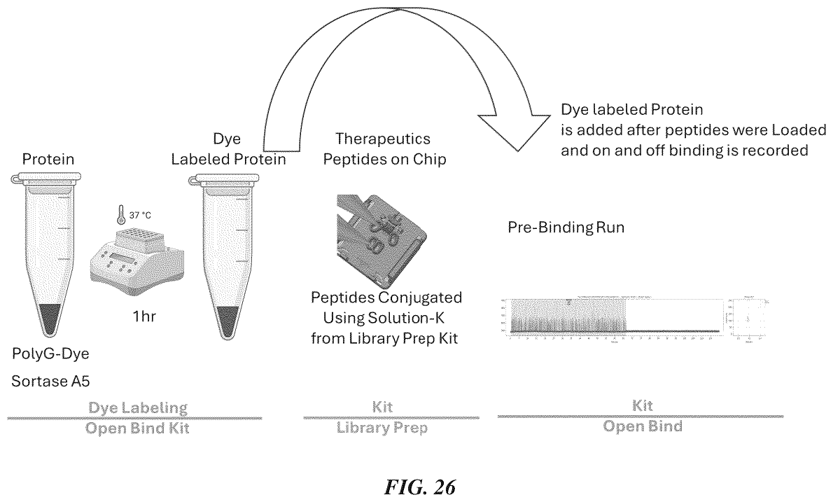

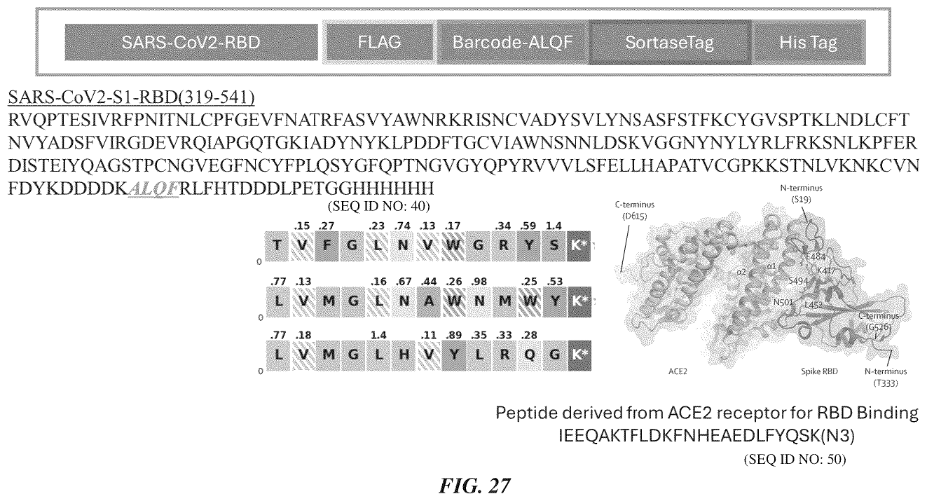

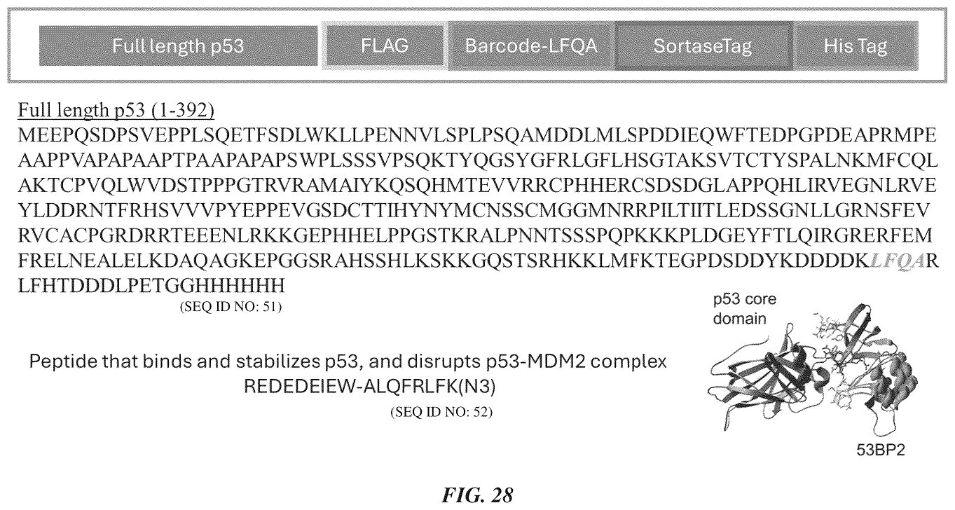

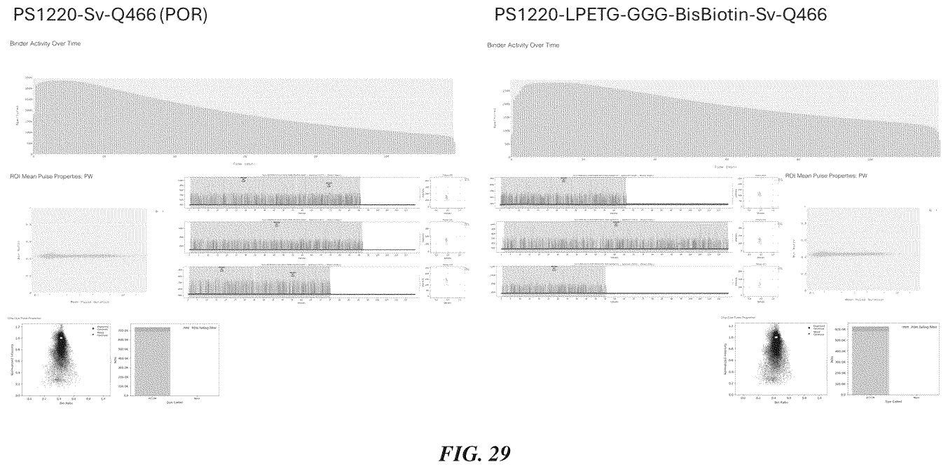

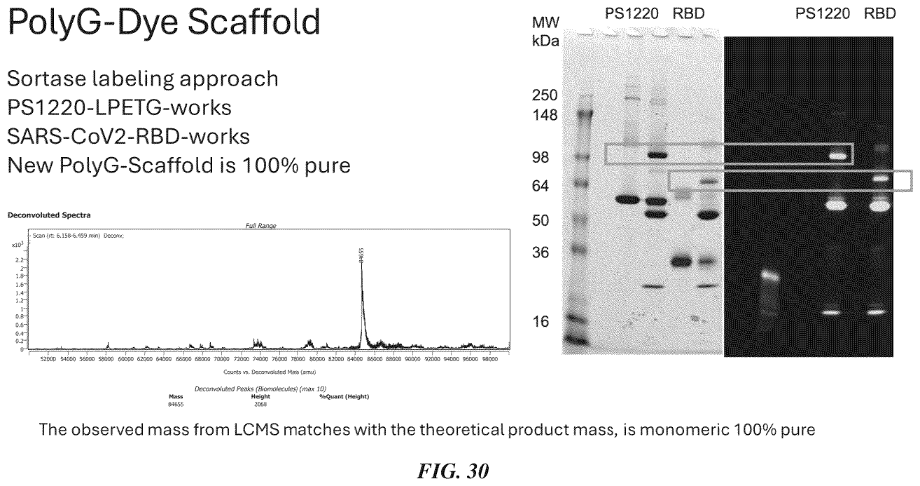

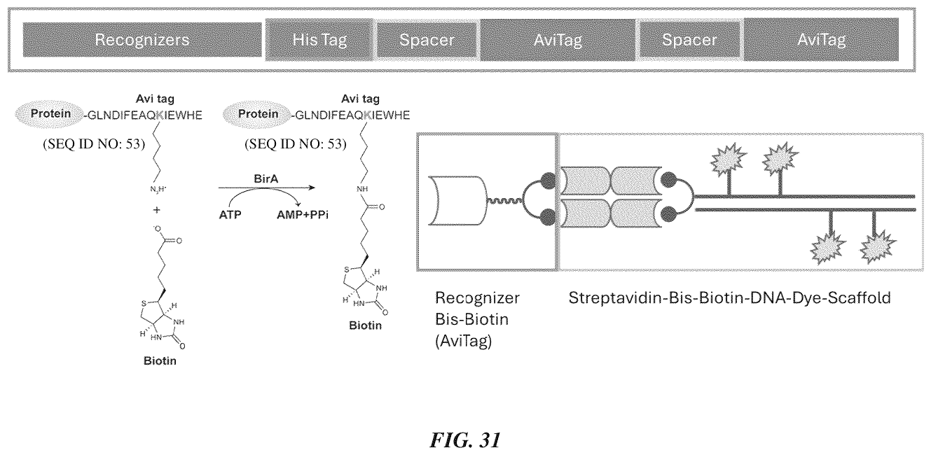

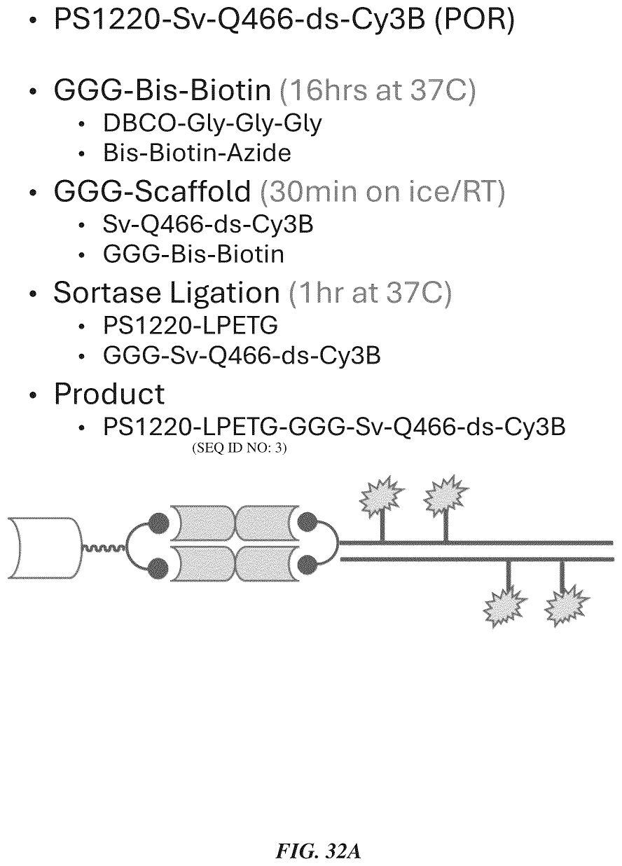

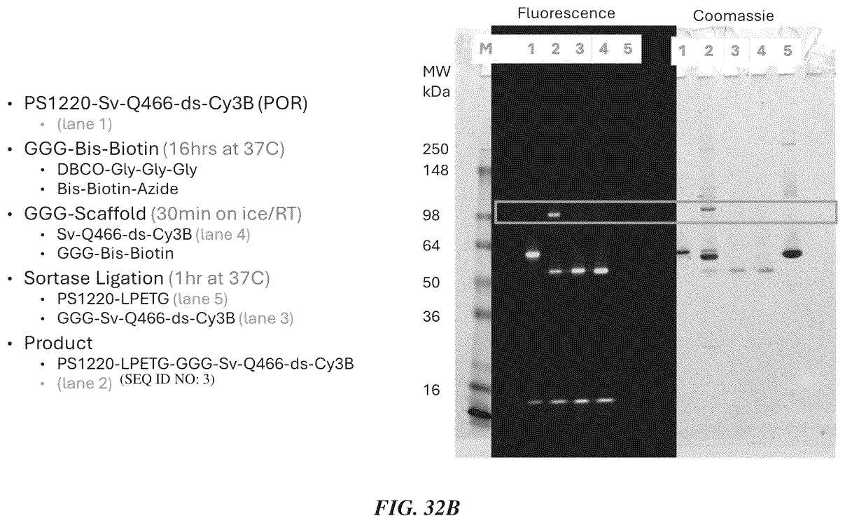

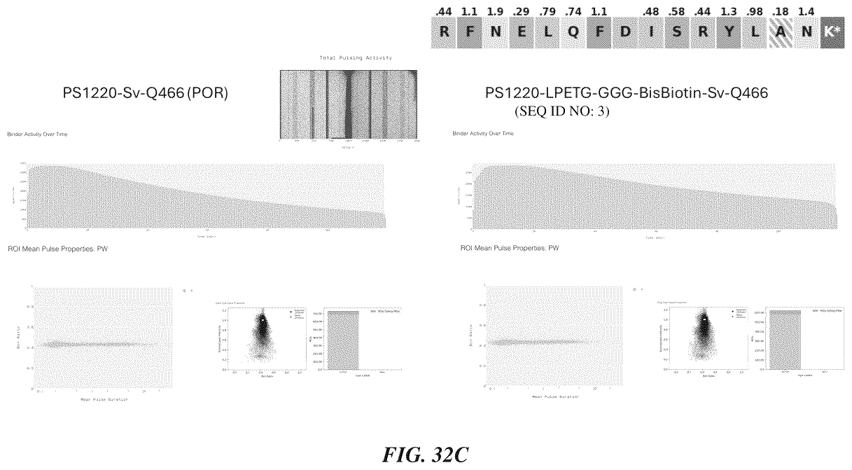

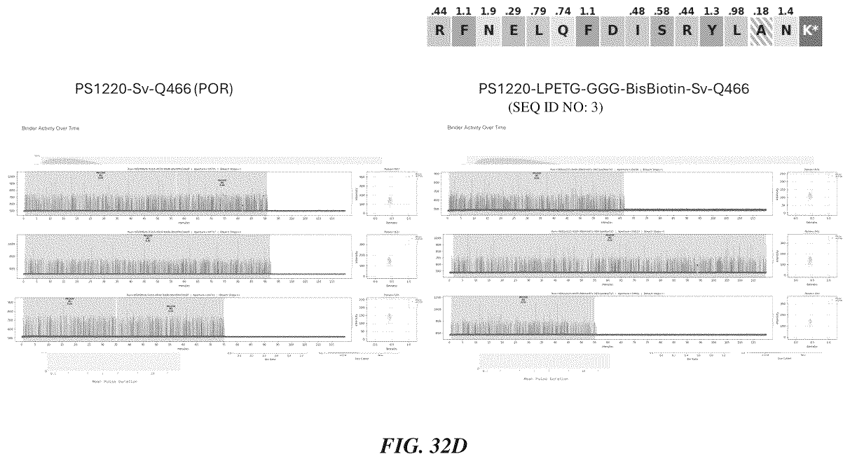

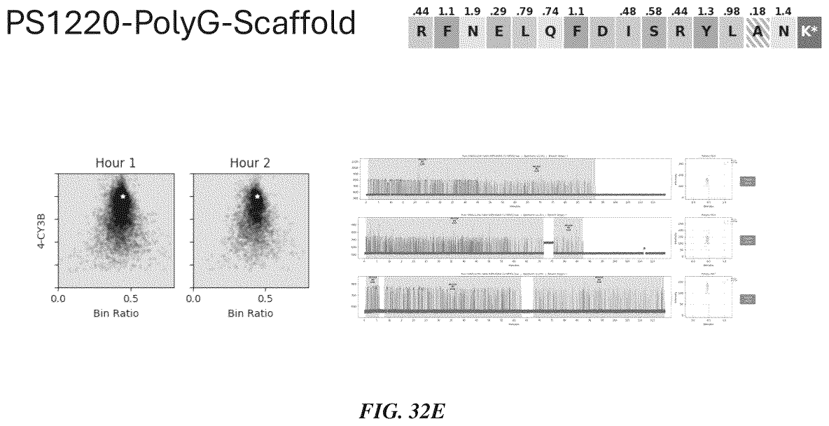

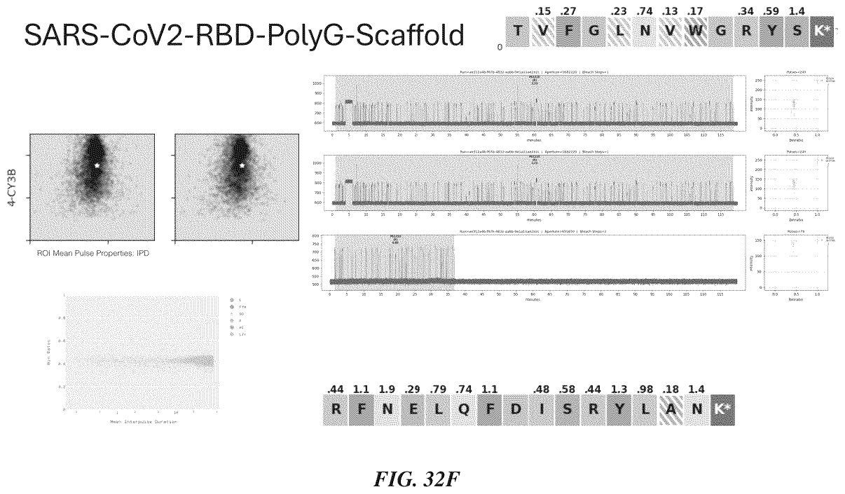

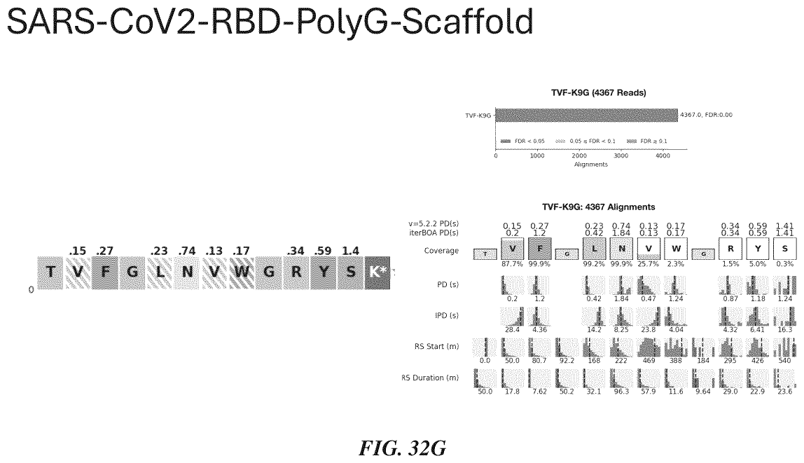

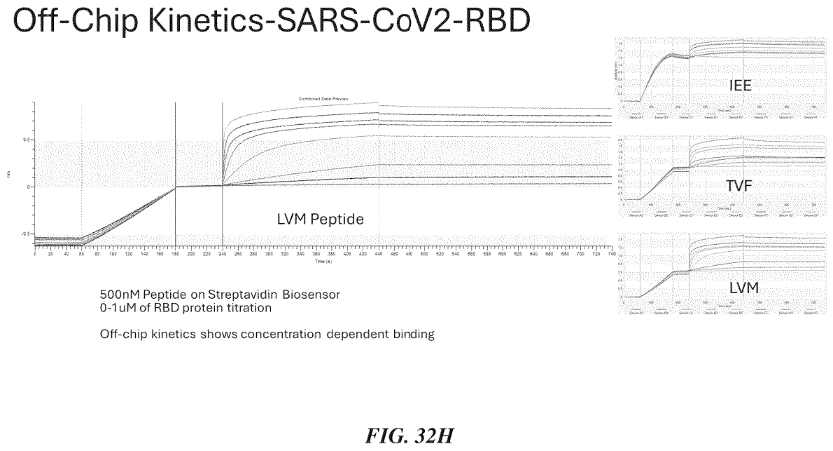

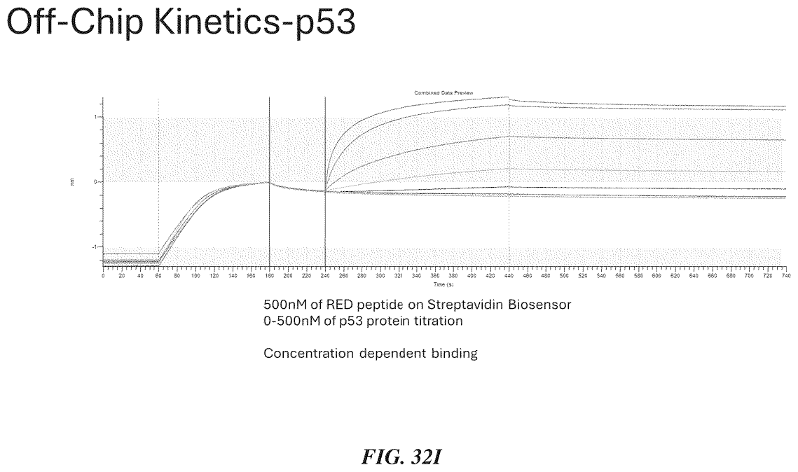

FIG. 23 shows an example workflow for the use of peptide barcodes to monitor protein expression. FIGS. 24A-24B show example constructs of dye-labeled polyG-linkers for use in labeling a POI according to the example workflow of FIG. 1B. FIG. 25 shows an example overview of dye-labeling POIs for detecting binding of POIs to peptides prior to peptide sequencing. FIG. 26 shows an example workflow for dye-labeling a POI and detecting binding of the labeled POI to peptides prior to peptide sequencing. FIG. 27 shows an example construct of a model protein (RBD) for dye-labeling as described herein. FIG. 28 shows an example construct of a model protein (p53) for dye-labeling as described herein. FIG. 29 shows experimental results demonstrating detection of binding between a target molecule and an affinity reagent (PS1220) labeled with a polyG-linker described herein. FIG. 30 shows experimental results demonstrating purity of the polyG-linker dye scaffold. FIG. 31 shows an example construct of a recognizer-AviTag-biotinylation-scaffold for dye-labeling with polyG-linkers described herein. FIGS. 32A-32I show experimental results for proteins that were dye-labeled using polyG-linkers described herein.

### Example 4. Peptide Screening for BCL2 Regulated Apoptotic Pathway

The intricate web of protein-protein interactions orchestrates both the health and demise of cells. BCL2, a member of the B-cell lymphoma-2 family, stands at the heart of cellular fate, regulating apoptosis through its selective binding to pro-apoptotic proteins. Among these, BID-a BH3-only protein interacts with BCL2 via its BH3 domain, a short, conserved peptide motif. Deciphering the details of BCL2's binding to the BID BH3 peptide is crucial for understanding apoptosis regulation and for the development of targeted therapeutics. The present disclosure relates, at least in part, to approaches for characterizing BCL2-BID BH3 peptide interactions, with a particular focus on the use of Next-Generation Peptide Sequencing (NGPS) technologies.

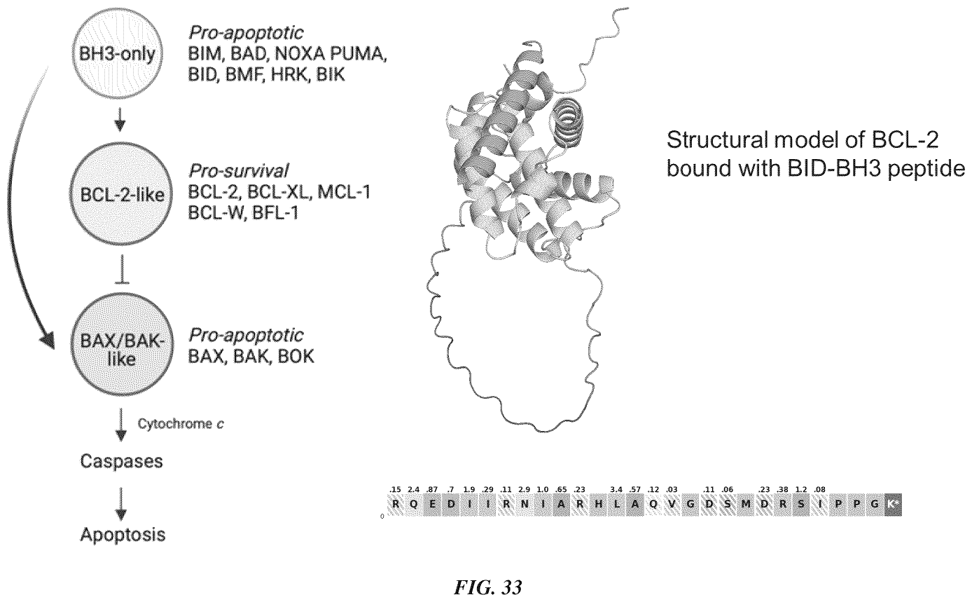

The BCL2 family is divided into pro-apoptotic and anti-apoptotic members, with their balance determining cell survival. BCL2 itself is anti-apoptotic, neutralizing pro-apoptotic signals by binding to BH3 domains of proteins such as BID, BAX, and BAD (FIG. 33). The BH3 domain is a helical motif approximately 16 amino acids long, necessary and sufficient for this interaction. The core consensus sequence of the BID BH3 domain enables high-affinity binding to a hydrophobic groove on BCL2.

Structural studies, including X-ray crystallography and NMR, have revealed that this interaction is mediated by a network of hydrophobic contacts and hydrogen bonds. Specific residues within the BID BH3 peptide make critical contacts with complementary sites on BCL2, driving selectivity and affinity.

The integration of BCL2-BID BH3 peptide binding assays with NGPS technology opens new avenues for research and provides several advantages:
Epitope Mapping: Identification of critical residues within the BH3 domain required for BCL2 binding;
Variant Screening: High-throughput analysis of peptide libraries to determine how sequence variations affect binding;
Drug discovery: Screen libraries of small molecules to identify potential inhibitors of Bcl-2/BID-BH3 interaction;
Mutagenesis studies: Investigate the impact of specific mutations in Bcl-2 or BID-BH3 on binding affinity; and
Kinetic studies: Adapt the assay to measure binding kinetics (on-rate and off-rate) under certain conditions.

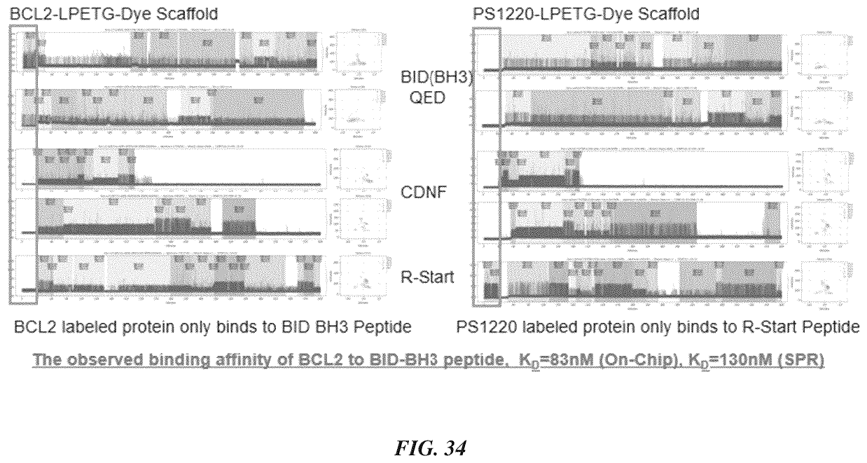

To test the BCL2 and BID interaction at single molecule level with prebinding followed by a dynamic sequencing, recombinant BCL2 construct was used to insert LPETG (SEQ ID NO: 3) tag at c-term and followed similar labeling and purification steps followed for R-recognizer. The labeled BCL2 protein was loaded on the chip during the prebinding step to screen against the target that has binding affinity and results in BCL2 pulsing on and off cycle. The CDNF library and the NAA arginine start peptide with a synthetic peptide derived from BID-BH3 protein were loaded on both side of the chip, and R-recognizer was added on one side of the chip and BCL2 on the other side of the chip. The R-recognizer only produced pulsing activities for R start peptide which is identified followed by dynamic sequencing. Similarly, the BCL2 protein produced pulsing activities on only BID-BH3 peptide, which was identified by the prebinding pulsing followed by dynamic sequencing, while none of the CDNF peptides or R start peptides resulted in positive prebinding for BCL2. The results demonstrated a unique approach to combine the affinity driven pre-binding followed by sequencing to therapeutically select the hits as shown in FIG. 34. The extracted binding affinity for BCL2 binding to BID BH3 peptide is KD=83 nM.

BCL2's binding to the BID BH3 peptide is a cornerstone of apoptotic regulation. Next generation protein sequencing is revolutionizing the landscape of peptide therapeutics discovery and provides unprecedented resolution for dissecting this interaction, by enabling direct, high-throughput screening, unbiased sequencing of peptide libraries and rational drug design. NGPS accelerates the identification of novel candidates with optimal properties for drug development. As NGPS technologies evolve, their integration into protein-peptide interaction studies promises to accelerate discoveries in cell biology, oncology, and therapeutics and the Pro Mode is set to become an indispensable tool in the pursuit of safer, more effective, and more diverse peptide-based medicines.

### EQUIVALENTS AND SCOPE

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the invention encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the invention or aspects of the invention consist, or consist essentially of, such elements and/or features. For purposes of simplicity, those embodiments have not been specifically set forth in haec verba herein.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03. It should be appreciated that embodiments described in this document using an open-ended transitional phrase (e.g., “comprising”) are also contemplated, in alternative embodiments, as “consisting of” and “consisting essentially of” the feature described by the open-ended transitional phrase. For example, if the application describes “a composition comprising A and B,” the application also contemplates the alternative embodiments “a composition consisting of A and B” and “a composition consisting essentially of A and B.”

Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the invention can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present invention, as defined in the following claims.

The recitation of a listing of chemical groups in any definition of a variable herein includes definitions of that variable as any single group or combination of listed groups. The recitation of an embodiment for a variable herein includes that embodiment as any single embodiment or in combination with any other embodiments or portions thereof. The recitation of an embodiment herein includes that embodiment as any single embodiment or in combination with any other embodiments or portions thereof.

1. A composition comprising:
an avidin protein comprising at least two biotin binding sites;
a first biotin moiety bound to at least one biotin binding site on the avidin protein; and
a polyglycine peptide attached to the first biotin moiety through a linker,
wherein the linker comprises a polyethylene glycol (PEG) moiety.

2. The composition of claim 1, wherein the polyglycine peptide comprises at least two glycine residues.

3. The composition of claim 1, wherein the polyglycine peptide comprises an N-terminal glycine residue having a free amino terminus.

4. The composition of claim 1, wherein the polyglycine peptide comprises a C-terminal glycine residue attached to the linker.

5. The composition of claim 4, wherein the C-terminal glycine residue is covalently attached to the PEG moiety.

6. (canceled)

7. The composition of claim 1, wherein the first biotin moiety comprises a bis-biotin moiety bound to two biotin binding sites on the avidin protein.

8. The composition of claim 1, wherein the avidin protein comprises streptavidin.

9. The composition of claim 1, wherein the avidin protein comprises at least one biotin binding site that is unbound.

10. The composition of claim 9, wherein the linker further comprises a nucleic acid, optionally wherein the first biotin moiety is covalently attached to the nucleic acid.

11. (canceled)

12. The composition of claim 10, wherein the PEG moiety forms a linkage group between the polyglycine peptide and the nucleic acid.

13. The composition of claim 10, wherein the linker further comprises a peptide forming a linkage group between the PEG moiety and the nucleic acid.

14. The composition of claim 13, wherein the peptide has an amino acid sequence of KFFDDDGGGDD (SEQ ID NO: 1), optionally wherein the linker-polyglycine comprises the formula of: X-PEG4-GGG, wherein: X is the peptide having the amino acid sequence of SEQ ID NO: 1; PEG4 is the PEG moiety, wherein the PEG moiety has four ethylene glycol subunits; and GGG is the polyglycine peptide, wherein the polyglycine peptide has three glycine residues.

15. The composition of claim 1, further comprising:
a second biotin moiety bound to at least one biotin binding site on the avidin protein; and
a nucleic acid attached to the second biotin moiety,
wherein the nucleic acid comprises one or more luminescent labels.

16. The composition of claim 15, wherein the one or more luminescent labels comprise at least two fluorophore dyes.

17. (canceled)

18. The composition of claim 15, wherein the first biotin moiety is covalently attached to the PEG moiety.

19. The composition of claim 15, wherein the second biotin moiety comprises a bis-biotin moiety bound to two biotin binding sites on the avidin protein.

20. The composition of claim 15, wherein:
the avidin protein comprises four biotin binding sites,
the first biotin moiety comprises a bis-biotin moiety bound to two biotin binding sites on the avidin protein, and
the second biotin moiety comprises a bis-biotin moiety bound to two biotin binding sites on the avidin protein.

21. A method comprising:
a) contacting a fusion polypeptide comprising a peptide barcode with the composition of claim 9; and
b) enzymatically conjugating the peptide barcode to the polyglycine peptide, thereby forming a conjugate comprising the peptide barcode and the avidin protein.

22-39. (canceled)

40. A method comprising:
a) contacting a fusion polypeptide comprising a protein of interest with the composition of claim 15; and
b) enzymatically conjugating the protein of interest to the polyglycine peptide, thereby forming a conjugate comprising the protein of interest and the one or more luminescent labels.

41-55. (canceled)

56. A fusion polypeptide, comprising a protein of interest, an affinity tag, a linker, a cleavage site, a barcode, and a sortase tag.

57-93. (canceled)


SEQUENCE LISTING (69 sequences)

SEQ ID NO: 1 - protein, 11 aa, synthetic construct
KFFDDDGGGDD

SEQ ID NO: 2 - protein, 5 aa, synthetic construct
LPXTG

SEQ ID NO: 3 - protein, 5 aa, synthetic construct
LPETG

SEQ ID NO: 4 - protein, 6 aa, synthetic construct
HHHHHH

SEQ ID NO: 5 - protein, 6 aa, synthetic construct
LPETGG

SEQ ID NO: 6 - protein, 10 aa, synthetic construct
GGGGSGGGGS

SEQ ID NO: 7 - protein, 5 aa, synthetic construct
DDDDK

SEQ ID NO: 8 - protein, 15 aa, synthetic construct
RFEQIANFAELPETG

SEQ ID NO: 9 - protein, 15 aa, synthetic construct
RQAELFRDYSLPETG

SEQ ID NO: 10 - protein, 15 aa, synthetic construct
FQRLAELEQALPETG

SEQ ID NO: 11 - protein, 15 aa, synthetic construct
FALRQDYVAQLPETG

SEQ ID NO: 12 - protein, 15 aa, synthetic construct
QRESFLFLNELPETG

SEQ ID NO: 13 - protein, 15 aa, synthetic construct
NDYRLSQRYLLPETG

SEQ ID NO: 14 - protein, 15 aa, synthetic construct
ALQRFEQDYSLPETG

SEQ ID NO: 15 - protein, 15 aa, synthetic construct
ELFNRALNAFLPETG

SEQ ID NO: 16 - protein, 35 aa, synthetic construct
DYKDDDDKGGGGSGGGGSKRFEQIANFAELPETGH

SEQ ID NO: 17 - protein, 35 aa, synthetic construct
DYKDDDDKGGGGSGGGGSKRQAELFRDYSLPETGH

SEQ ID NO: 18 - protein, 35 aa, synthetic construct
DYKDDDDKGGGGSGGGGSKFQRLAELEQALPETGH

SEQ ID NO: 19 - protein, 35 aa, synthetic construct
DYKDDDDKGGGGSGGGGSKFALRQDYVAQLPETGH

SEQ ID NO: 20 - protein, 35 aa, synthetic construct
DYKDDDDKGGGGSGGGGSKQRESFLFLNELPETGH

SEQ ID NO: 21 - protein, 35 aa, synthetic construct
DYKDDDDKGGGGSGGGGSKNDYRLSQRYLLPETGH

SEQ ID NO: 22 - protein, 35 aa, synthetic construct
DYKDDDDKGGGGSGGGGSKALQRFEQDYSLPETGH

SEQ ID NO: 23 - protein, 35 aa, synthetic construct
DYKDDDDKGGGGSGGGGSKELFNRALNAFLPETGH

SEQ ID NO: 24 - protein, 8 aa, synthetic construct
DYKDDDDK

SEQ ID NO: 25 - protein, 37 aa, synthetic construct
DYKDDDDKGGGGSGGGGSKALQFRLFHTDDDLPETGH

SEQ ID NO: 26 - protein, 26 aa, synthetic construct
DYKDDDDKALQFRLFHTDDDLPETGH

SEQ ID NO: 27 - protein, 12 aa, synthetic construct
ALQFRLFHTDDD

SEQ ID NO: 28 - protein, 12 aa, synthetic construct
LFQARLFHTDDD

SEQ ID NO: 29 - protein, 9 aa, synthetic construct
YPYDVPDYA

SEQ ID NO: 30 - protein, 10 aa, synthetic construct
EQKLISEEDL

SEQ ID NO: 31 - protein, 14 aa, synthetic construct
GKPIPNPLLGLDST

SEQ ID NO: 32 - protein, 5 aa, synthetic construct
RLIFA

SEQ ID NO: 33 - protein, 4 aa, synthetic construct
LIFA

SEQ ID NO: 34 - protein, 43 aa, synthetic construct
DYKDDDDKGGGGSGGGGSKALQFRLFHTDDDLPETGGHHHHHH

SEQ ID NO: 35 - protein, 42 aa, synthetic construct
DYKDDDDKGGGGSGGGGSKEYSRINAQDLRFLPETGHHHHHH

SEQ ID NO: 36 - protein, 4 aa, synthetic construct
QIRY

SEQ ID NO: 37 - protein, 6 aa, synthetic construct
RLFNAE

SEQ ID NO: 38 - protein, 6 aa, synthetic construct
LRFNAE

SEQ ID NO: 39 - protein, 6 aa, synthetic construct
FRAENL

SEQ ID NO: 40 - protein, 255 aa, synthetic construct
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFDYKDDDDKALQFRLFHTDDDLPETGGHHHHHH

SEQ ID NO: 41 - protein, 32 aa, synthetic construct
DYKDDDDKALQFRLFHTDDDLPETGGHHHHHH

SEQ ID NO: 42 - protein, 32 aa, synthetic construct
DYKDDDDKLFQARLFHTDDDLPETGGHHHHHH

SEQ ID NO: 43 - protein, 27 aa, synthetic construct
DYKDDDDKELRAQFAYPDDDLPETGGH

SEQ ID NO: 44 - protein, 22 aa, synthetic construct
DYKDDDDKEFLNRFYLPETGGH

SEQ ID NO: 45 - protein, 27 aa, synthetic construct
DYKDDDDKALQFRLFHTDDDLPETGGH

SEQ ID NO: 46 - protein, 13 aa, synthetic construct
TVFGLNVWKRYSK

SEQ ID NO: 47 - protein, 13 aa, synthetic construct
LVMGLNAWNMWYK

SEQ ID NO: 48 - protein, 13 aa, synthetic construct
LVMGLHVYLRQGK

SEQ ID NO: 49 - protein, 13 aa, synthetic construct
LVMGLNVWLRYSK

SEQ ID NO: 50 - protein, 27 aa, synthetic construct
IEEQAKTFLDKFNHEAEDLFYQSKNNN

SEQ ID NO: 51 - protein, 425 aa, synthetic construct
MEEPQSDPSVEPPLSQETFSDLWKLLPENNVLSPLPSQAMDDLMLSPDDIEQWFTEDPGPDEAPRMPEAAPPVAPAPAAPTPAAPAPAPSWPLSSSVPSQKTYQGSYGFRLGFLHSGTAKSVTCTYSPALNKMFCQLAKTCPVQLWVDSTPPPGTRVRAMAIYKQSQHMTEVVRRCPHHERCSDSDGLAPPQHLIRVEGNLRVEYLDDRNTFRHSVVVPYEPPEVGSDCTTIHYNYMCNSSCMGGMNRRPILTIITLEDSSGNLLGRNSFEVRVCACPGRDRRTEEENLRKKGEPHHELPPGSTKRALPNNTSSSPQPKKKPLDGEYFTLQIRGRERFEMFRELNEALELKDAQAGKEPGGSRAHSSHLKSKKGQSTSRHKKLMFKTEGPDSDDTKDDDDKLFQARLFHTDDDLPETGGHHHHHH

SEQ ID NO: 52 - protein, 20 aa, synthetic construct
REDEDEIEWALQFRLFKNNN

SEQ ID NO: 53 - protein, 15 aa, synthetic construct
GLNDIFEAQKIEWHE

SEQ ID NO: 54 - protein, 37 aa, synthetic construct
DYKDDDDKGGGGSGGGGSKAIQLRDSERYNFLPETGH

SEQ ID NO: 55 - protein, 37 aa, synthetic construct
DYKDDDDKGGGGSGGGGSKDARLYNRESIQFLPETGH

SEQ ID NO: 56 - protein, 37 aa, synthetic construct
DYKDDDDKGGGGSGGGGSKEIQRADSYLRNFLPETGH

SEQ ID NO: 57 - protein, 37 aa, synthetic construct
DYKDDDDKGGGGSGGGGSKELSIYARDNRQFLPETGH

SEQ ID NO: 58 - protein, 37 aa, synthetic construct
DYKDDDDKGGGGSGGGGSKERQRDSYINLAFLPETGH

SEQ ID NO: 59 - protein, 37 aa, synthetic construct
DYKDDDDKGGGGSGGGGSKERSRDLAYINFQLPETGH

SEQ ID NO: 60 - protein, 37 aa, synthetic construct
DYKDDDDKGGGGSGGGGSKESLRANIYDRQFLPETGH

SEQ ID NO: 61 - protein, 37 aa, synthetic construct
DYKDDDDKGGGGSGGGGSKEYSRINAQDLRFLPETGH

SEQ ID NO: 62 - protein, 37 aa, synthetic construct
DYKDDDDKGGGGSGGGGSKINEQARDLSYRFLPETGH

SEQ ID NO: 63 - protein, 37 aa, synthetic construct
DYKDDDDKGGGGSGGGGSKNAQYDRELSRIFLPETGH

SEQ ID NO: 64 - protein, 37 aa, synthetic construct
DYKDDDDKGGGGSGGGGSKNYQRDLESIRAFLPETGH

SEQ ID NO: 65 - protein, 37 aa, synthetic construct
DYKDDDDKGGGGSGGGGSKRDALQIERSYNFLPETGH

SEQ ID NO: 66 - protein, 37 aa, synthetic construct
DYKDDDDKGGGGSGGGGSKRENAQSYDIRLFLPETGH

SEQ ID NO: 67 - protein, 37 aa, synthetic construct
DYKDDDDKGGGGSGGGGSKRLEQRDSYINAFLPETGH

SEQ ID NO: 68 - protein, 37 aa, synthetic construct
DYKDDDDKGGGGSGGGGSKRLEYSDRINAQFLPETGH

SEQ ID NO: 69 - protein, 37 aa, synthetic construct
DYKDDDDKGGGGSGGGGSKRNRQESYLDAIFLPETGH